(19) United States
(12) Patent Application Publication
(10) Pub. No.: US 2024/0067940 A1
(43) Pub. Date: Feb. 29, 2024

(54) METHODS AND COMPOSITIONS FOR EDITING NUCLEOTIDE SEQUENCES

(71) Applicant: Prime Medicine, Inc., Cambridge, MA (US)

(72) Inventors: Christopher WILSON, Waltham, MA (US); Andrew V. ANZALONE, Cambridge, MA (US); Jonathan M. LEVY, Brookline, MA (US)

(21) Appl. No.: 18/478,179

(22) Filed: Sep. 29, 2023

Related U.S. Application Data
(63) Continuation of application No. PCT/US2022/023175, filed on Apr. 1, 2022.
(60) Provisional application No. 63/169,725, filed on Apr. 1, 2021.
(60) Provisional application No. 63/282,945, filed on Nov. 24, 2021.

(51) Int. Cl. C12N 9/12; C12N 9/22; C12N 15/11

(52) U.S. Cl. CPC C12N 9/1252; C12N 9/22; C12N 15/11; C12N 9/1276; C12Y 207/07049; C12Y 207/07007; C12N 2310/20; C12N 2800/80

(57) ABSTRACT

Disclosed herein, are prime editors for prime editing. Also disclosed are engineered reverse transcriptases and methods of using same for prime editing.

### CROSS-REFERENCE

This application is a continuation of International Application No. PCT/US2022/023175, filed Apr. 1, 2022, which claims the benefit of U.S. Provisional Application No. 63/169,725, filed Apr. 1, 2021; and U.S. Provisional Application No. 63/282,945, filed Nov. 24, 2021, each of which applications are incorporated herein by reference in their entirety.

### SEQUENCE LISTING

The instant application contains a Sequence Listing which has been submitted electronically in XML format and is hereby incorporated by reference in its entirety. Said XML copy, created on Sep. 28, 2023, is named 59761-719301-SL.xml and is 112,129 bytes in size.

### BACKGROUND OF THE DISCLOSURE

Modern therapeutic manipulations or biotechnological development entails effective genome editing. An effective genome editing technique needs to be accurate, capable of delivering a desired nucleotide change at a chosen genome location without undesirable changes at locations other than the chosen genome location. An effective genome editing technique also needs to be customable; modulable; and programmable, suitable of making any genome changes in any cells or organisms. Furthermore, an effective genome editing technique needs to scalable and reliable, proficient in making any genome changes reproducibly in a robust scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### SUMMARY OF THE DISCLOSURE

In some embodiments, the present disclosure provides a prime editing composition that comprises a) a DNA binding domain or a polynucleotide encoding the DNA binding domain; and b) a DNA polymerase domain or a polynucleotide encoding the DNA polymerase domain, wherein the DNA polymerase domain comprises an amino acid sequence with at least 85% sequence identity to a sequence selected from the group consisting of SEQ ID NOs: 5, 6, 13, 15, 16, 17, 18, 21, 22, 130, 131, 204, 230, 232-244, 249-257, 261, 270, 271, 327, 329, 332, 333, 337, 340, 341, 342, 344, 489, 990-1006, 209, 210, 231, and 229. In some embodiments, wherein the DNA polymerase domain comprises an amino acid sequence with at least 85% sequence identity to any one of sequences set forth in SEQ ID NOs: 209, 210, 229-244, 249-257, 261, 270, 271, 329, 990-1006.

In some embodiments, wherein the amino acid sequence of the DNA polymerase domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence is SEQ ID NO: 261. In some embodiments, the selected sequence for the DNA polymerase domain is SEQ ID NO:270. In some embodiments, the selected sequence for the DNA polymerase domain is SEQ ID NO:16. In some embodiments, the selected sequence for the DNA polymerase domain is SEQ ID NO:18. In some embodiments, the DNA binding domain comprises a CRISPR associated (Cas) protein. In some embodiments, the Cas protein is a Type II Cas protein. In some embodiments, the Cas protein is a Cas9 protein. In some embodiments, the Cas9 protein is a nickase. In some embodiments, the Cas9 protein comprises a mutation in a HNH domain. In some embodiments, the Cas protein is a Type V Cas protein.

In some embodiments, the Cas protein is a Cas12a, Cas12b, Cas12c, Cas12d, or Cas12e. In some embodiments, the Cas protein is a Cas12b. In some embodiments, the DNA binding domain comprises an amino acid sequence with at least 85% sequence identity to a sequence selected from the group consisting of SEQ ID NOs: 138-146, 494, 858, 1100, 495-503, 1011, 1013. In some embodiments, the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA binding domain is SEQ ID NOs: 495-503, 1011, 1013, or 1100. In some embodiments, the selected sequence for the DNA binding domain is SEQ ID NOs: 495. In some embodiments, the selected sequence for the DNA binding domain is SEQ ID NOs: 496. In some embodiments, the selected sequence for the DNA binding domain is SEQ ID NOs: 501. In some embodiments, the selected sequence for the DNA binding domain is SEQ ID NO: 502. In some embodiments, the DNA binding domain is connected to the DNA polymerase domain by a linker. In some embodiments, the DNA binding domain is connected to the DNA polymerase domain by a peptide linker in a fusion protein. In some embodiments, the peptide linker comprises a sequence selected from the group consisting of SEQ ID NOs: 272-318, 1014. In some embodiments, the fusion protein comprises the DNA polymerase and the DNA binding domain from N-terminus to C-Terminus. In some embodiments, the fusion protein comprises the DNA binding and the DNA polymerase domain from N-terminus to C-Terminus. In some embodiments, the DNA polymerase domain, the DNA binding domain, or both comprise one or more nuclear localization signals. In some embodiments, the primer editing composition further comprises a solubility-enhancement (SET) domain. In some embodiments, the SET domain comprises an amino acids sequence selected from the group consisting of SEQ ID NOs: 96-124, 137. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a prime editing composition that comprises a fusion protein, or a polynucleotide encoding the fusion protein, wherein the fusion protein comprises a DNA binding domain and a DNA polymerization domain connected via a peptide linker, wherein the peptide linker comprises an amino acid sequence with at least 80% sequence identity to a sequence selected from the group consisting of SEQ ID NO: 273-318. In some embodiments, the amino acid sequence of the peptide linker has at least about 81%, 82%, 83%, 84%, 85%, 86%, 87%, 88%, 80%, 90%, 91%, 92%, 93%, 94%, 95%, 96%, 97%, 98%, 99%, or 100% sequence identity to the selected sequence. In some embodiments, the DNA polymerase domain comprises an amino acid sequence with at least 85% sequence identity to SEQ ID NO:856 or SEQ ID NO: 884. In some embodiments, the amino acid sequence of the DNA polymerase domain has at least about 86%, 87%, 88%, 80%, 90%, 91%, 92%, 93%, 94%, 95%, 96%, 97%, 98%, 99%, or 100% sequence identity to SEQ ID NO: 856. In some embodiments, the Cas protein is a Type II Cas protein.

In some embodiments, the Cas protein is a Cas9 protein. In some embodiments, the Cas9 protein is a nickase.

In some embodiments, the Cas9 protein comprises a mutation in a HNH domain. In some embodiments, the Cas protein is a Type V Cas protein. In some embodiments, the Cas protein is a Cas12a, Cas12b, Cas12c, Cas12d, or Cas12e. In some embodiments, the Cas protein is a Cas12b. In some embodiments, the DNA binding domain comprises an amino acid sequence with at least 85% sequence identity to a sequence selected from the group consisting of SEQ ID NOs: 138-146, 494, 858, 1100, 1011, 1013, 495-503. In some embodiments, the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence.

In some embodiments, the selected sequence for the DNA binding domain is SEQ ID NOs: 495-503, 1011, 1013, 1100. In some embodiments, the selected sequence for the DNA binding domain is SEQ ID NOs: 495.

In some embodiments, the selected sequence for the DNA binding domain is SEQ ID NOs: 496. In some embodiments, the selected sequence for the DNA binding domain is SEQ ID NOs: 501. In some embodiments, the selected sequence for the DNA binding domain is SEQ ID NO: 502. In some embodiments, the fusion protein comprises the DNA polymerase and the DNA binding domain from N-terminus to C-Terminus. In some embodiments, the fusion protein comprises the DNA binding and the DNA polymerase domain from N-terminus to C-Terminus. In some embodiments, the DNA polymerase domain, the DNA binding domain, or both comprise one or more nuclear localization signals. In some embodiments, the primer editing composition further comprises a solubility-enhancement (SET) domain. In some embodiments, the SET domain comprises an amino acids sequence selected from the group consisting of SEQ ID NOs: 96-124, 137. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a prime editing composition that comprises a DNA binding domain, or a polynucleotide encoding the DNA binding domain, wherein the DNA binding domain comprises an amino acid sequence with at least 80% sequence identity to a sequence selected from the group consisting of SEQ ID NOs: 496, 501, 502, 1011, and 1013; and a DNA polymerase domain or a polynucleotide encoding the DNA polymerase domain. In some embodiments, the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA binding domain is SEQ ID NOs: 496. In some embodiments, the selected sequence for the DNA binding domain is SEQ ID NOs: 501. In some embodiments, the selected sequence for the DNA binding domain is SEQ ID NOs: 502. In some embodiments, the DNA polymerase domain comprises an amino acid sequence with at least 85% sequence identity to SEQ ID NO:856 or SEQ ID NO: 884. In some embodiments, the amino acid sequence of the DNA polymerase domain has at least about 86%, 87%, 88%, 80%, 90%, 91%, 92%, 93%, 94%, 95%, 96%, 97%, 98%, 99%, or 100% sequence identity to SEQ ID NO: 856. In some embodiments, the DNA binding domain is connected to the DNA polymerase domain by a linker. In some embodiments, the DNA binding domain is connected to the DNA polymerase domain by a peptide linker in a fusion protein. In some embodiments, the peptide linker comprises a sequence selected from the group consisting of 272-318, 1014. In some embodiments, the fusion protein comprises the DNA polymerase and the DNA binding domain from N-terminus to C-Terminus. In some embodiments, the fusion protein comprises the DNA binding and the DNA polymerase domain from N-terminus to C-Terminus. In some embodiments, the DNA polymerase domain, the DNA binding domain, or both comprise one or more nuclear localization signals. In some embodiments, the primer editing composition further comprises a solubility-enhancement (SET) domain. In some embodiments, the SET domain comprises an amino acids sequence selected from the group consisting of SEQ ID NOs: 96-124, 137. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a prime editing composition that comprises a DNA binding domain or a polynucleotide encoding the DNA binding domain; and a DNA polymerase domain, or a polynucleotide encoding the DNA polymerase domain, wherein the DNA polymerase domain comprises an amino acid sequence with at least 85% sequence identity to a sequence selected form the group consisting of SEQ ID NOs: 81, 91, 82, 84. In some embodiments, the amino acid sequence of the DNA polymerase domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA polymerase domain is SEQ ID NO: 81. In some embodiments, the selected sequence for the DNA polymerase domain is SEQ ID NO: 91. In some embodiments, the selected sequence for the DNA polymerase domain is SEQ ID NO: 82. In some embodiments, the selected sequence for the DNA polymerase domain is SEQ ID NO: 84. In some embodiments, the DNA binding domain comprises a CRISPR associated (Cas) protein.

In some embodiments, the Cas protein is a Type II Cas protein. In some embodiments, the Cas protein is a Cas9 protein. In some embodiments, the Cas9 protein is a nickase. In some embodiments, the Cas9 protein comprises a mutation in a HNH domain. In some embodiments, the Cas protein is a Type V Cas protein.

In some embodiments, the Cas protein is a Cas12a, Cas12b, Cas12c, Cas12d, or Cas12e. In some embodiments, the Cas protein is a Cas12b. In some embodiments, the DNA binding domain comprises an amino acid sequence with at least 85% sequence identity to a sequence selected from the group consisting of SEQ ID NOs: 138-146, 494, 858, 1100, 1011, 1013, 495-503. In some embodiments, the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA binding domain is SEQ ID NOs: 495-503, 1011, 1013, 1100. In some embodiments, the selected sequence for the DNA binding domain is SEQ ID NOs: 495. In some embodiments, the selected sequence for the DNA binding domain is SEQ ID NOs: 496. In some embodiments, the selected sequence for the DNA binding domain is SEQ ID NOs: 501. In some embodiments, the selected sequence for the DNA binding domain is SEQ ID NO:502. In some embodiments, the DNA binding domain is connected to the DNA polymerase domain by a linker. In some embodiments, the DNA binding domain is connected to the DNA polymerase domain by a peptide linker in a fusion protein. In some embodiments, the peptide linker comprises a sequence selected from the group consisting of 272-318, 1014. In some embodiments, the fusion protein comprises the DNA polymerase and the DNA binding domain from N-terminus to C-Terminus. In some embodiments, the fusion protein comprises the DNA binding and the DNA polymerase domain from N-terminus to C-Terminus. In some embodiments, the DNA polymerase domain, the DNA binding domain, or both comprise one or more nuclear localization signals. In some embodiments, the primer editing composition further comprises a solubility-enhancement (SET) domain. In some embodiments, the SET domain comprises an amino acids sequence selected from the group consisting of SEQ ID NOs: 96-124, 137. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a prime editing composition comprising a DNA binding domain or a polynucleotide encoding the DNA binding domain, and a reverse transcriptase (RT) domain or a polynucleotide encoding the RT domain, wherein the RT domain is from a naturally occurring fusion between a Type III CRISPR system protein and a reverse transcriptase, and wherein the DNA binding domain is heterologous to the RT domain. In some embodiments, the RT domain is from a naturally occurring Cas1-RT fusion protein. In some embodiments, the RT domain comprises an amino acid sequence with at least 85% sequence identity to a sequence selected from the group consisting of SEQ ID NOs: 345, 129-136, 396, 533-846. In some embodiments, the amino acid sequence of the RT domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence for the RT domain is SEQ ID NO: 209.

In some embodiments, the selected sequence for the RT domain is SEQ ID NO: 210. In some embodiments, the RT domain is fused directly to the DNA binding domain. In some embodiments, the RT domain is fused to the N-terminus of the DNA binding domain. In some embodiments, the RT domain is fused to the C-terminus of the DNA binding domain. In some embodiments, the DNA binding domain comprises a CRISPR associated (Cas) protein. In some embodiments, the Cas protein is a Type II Cas protein. In some embodiments, the Cas protein is a Cas9 protein. In some embodiments, the Cas9 protein is a nickase.

In some embodiments, the Cas9 protein comprises a mutation in a HNH domain. In some embodiments, the Cas protein is a Type V Cas protein. In some embodiments, the Cas protein is a Cas12a, Cas12b, Cas12c, Cas12d, or Cas12e. In some embodiments, the Cas protein is a Cas12b. In some embodiments, the DNA binding domain comprises an amino acid sequence with at least 85% sequence identity to a sequence selected from the group consisting of SEQ ID NOs: 138-146, 494, 858, 1100, 1011, 1013, 495-503. In some embodiments, the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA binding domain is SEQ ID NOs: 495-503, 1100, 1011, 1013. In some embodiments, the selected sequence for the DNA binding domain is SEQ ID NOs: 1011. In some embodiments, the selected sequence for the DNA binding domain is SEQ ID NOs: 1013

In some embodiments, the selected sequence for the DNA binding domain is SEQ ID NOs: 496. In some embodiments, the selected sequence for the DNA binding domain is SEQ ID NOs: 501. In some embodiments, the selected sequence for the DNA binding domain is SEQ ID NO: 502. In some embodiments, the RT domain, the DNA binding domain, or both comprise one or more nuclear localization signals. In some embodiments, the primer editing composition further comprises a solubility-enhancement (SET) domain. In some embodiments, the SET domain comprises an amino acids sequence selected from the group consisting of SEQ ID NOs: 96-124, 137. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a prime editing composition that comprises a DNA polymerase domain or a polynucleotide encoding the DNA polymerase domain, wherein the DNA polymerase domain comprises an amino acid sequence having at least 85% identity to SEQ ID NO: 856 or 884; a DNA binding domain or a polynucleotide encoding the DNA binding domain, wherein the DNA binding domain comprises an amino acid sequence having at least 85% identity to SEQ ID NO: 1011 or 1013; and a solubility-enhancement (SET) domain or a polynucleotide encoding the SET domain, wherein the SET domain comprises an amino acid sequence with at least 80% sequence identity to an amino acid sequence selected from the group consisting of SEQ ID NOs: 96-124, 137. In some embodiments, the amino acid sequence for the SET domain has at least 81%, 82%, 83%, 84%, 85%, 86%, 87%, 88%, 80%, 90%, 91%, 92%, 93%, 94%, 95%, 96%, 97%, 98%, 99%, or 100% sequence identity to the selected sequence. In some embodiments, the selected sequence for the SET domain is SEQ ID NO: 102. In some embodiments, the selected sequence for the SET domain is SEQ ID NO: 137. In some embodiments, the amino acid sequence for the DNA polymerase domain has at least 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA polymerase domain is 856. In some embodiments, the selected sequence for the DNA polymerase domain is 884. In some embodiments, the amino acid sequence for the DNA binding domain has at least 86%, 87%, 88%, 80%, 90%, 91%, 92%, 93%, 94%, 95%, 96%, 97%, 98%, 99%, or 100% sequence identity to the selected sequence. In some embodiments, the selected sequence for the DNA binding domain is SEQ ID NO: 1011. In some embodiments, the selected sequence for the DNA binding domain is SEQ ID NO: 1013. In some embodiments, the SET domain is fused to the DNA polymerase via an SGGS linker. In some embodiments, the DNA binding domain is connected to the DNA polymerase domain by a linker. In some embodiments, the DNA binding domain is connected to the DNA polymerase domain by a peptide linker in a fusion protein. In some embodiments, the peptide linker comprises a sequence selected from the group consisting of SEQ ID NOs: 272-318, 1014. In some embodiments, the fusion protein comprises the DNA polymerase and the DNA binding domain from N-terminus to C-Terminus. In some embodiments, the fusion protein comprises the DNA binding and the DNA polymerase domain from N-terminus to C-Terminus. In some embodiments, the DNA polymerase domain, the DNA binding domain, the SET domain, or a combination thereof comprise one or more nuclear localization signals. In some embodiments, the fusion protein comprises a nuclear localization signal, the DNA binding domain, the peptide linker, the DNA polymerase domain, the SGGS linker, the SET domain, and a second nuclear localization signal from N-terminus to C-terminus. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the prime editing composition further comprises a prime editing guide RNA (PEgRNA), or a polynucleotide encoding the PEgRNA. In some embodiments, the prime editing composition further comprises a nick guide RNA (ngRNA), or a polynucleotide encoding the ngRNA.

In some embodiments, the present disclosure provides a vector comprising one or more of the polynucleotides of the prime editing compositions of the present disclosure. In some embodiments, the vector is a AAV vector. In some embodiments, the vector is an lipid nanoparticle (LNP).

In some embodiments, the present disclosure provides a pharmaceutical composition comprising the prime editing composition of the present disclosure, or the vector of the present disclosure. In some embodiments, the pharmaceutical composition further comprises a pharmaceutically acceptable excipient.

In some embodiments, the present disclosure provides an engineered reverse transcriptase (RT) that comprises an amino acid sequence with at least 85% sequence identity to an amino acid sequence selected from the group consisting of SEQ ID NOs: 81-95. In some embodiments, the amino acid sequence for the engineered RT has at least 86%, 87%, 88%, 80%, 90%, 91%, 92%, 93%, 94%, 95%, 96%, 97%, 98%, 99%, or 100% sequence identity to the selected sequence. In some embodiments, the selected sequence for the engineered RT is SEQ ID NO: 84. In some embodiments, the selected sequence for the engineered RT is SEQ ID NO: 82. In some embodiments, the selected sequence for the engineered RT is SEQ ID NO: 81

In some embodiments, the selected sequence for the engineered RT is SEQ ID NO: 91 In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a prime editing composition that comprises a fusion protein, or a polynucleotide encoding the fusion protein, wherein the fusion protein comprises a DNA binding domain and a DNA polymerization domain connected via a peptide linker, wherein the fusion protein comprises an amino acid sequence with at least 80% sequence identity to a sequence selected from the group consisting of SEQ ID NO: 504, 939-987, 1011, 1012, 1013, 1007-1010, 504-513, 514-521. In some embodiments, the amino acid sequence of the DNA polymerase domain has at least about 86%, 87%, 88%, 80%, 90%, 91%, 92%, 93%, 94%, 95%, 96%, 97%, 98%, 99%, or 100% sequence identity to the selected sequence. In some embodiments, the selected sequence is SEQ ID NO: 940. In some embodiments, the selected sequence is SEQ ID NO: 941. In some embodiments, the selected sequence is SEQ ID NO: 976.

In some embodiments, the selected sequence is SEQ ID NO: 977. In some embodiments, the selected sequence is SEQ ID NO: 505. In some embodiments, the selected sequence is SEQ ID NO: 511.

In some embodiments, the selected sequence is SEQ ID NO: 512. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the present disclosure provides a vector comprising one or more of the polynucleotides of the prime editing compositions of the present disclosure. In some embodiments, the vector is a AAV vector. In some embodiments, the vector is an lipid nanoparticle (LNP).

In some embodiments, the present disclosure provides a pharmaceutical composition comprising the prime editing composition of the present disclosure, or the vector of the present disclosure.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 a cartoon illustration of the domain structure of an exemplary prime editor comprising a DNA binding domain that is a Cas protein domain and a DNA polymerase domain (e.g., a reverse transcriptase domain) connected by a linker.

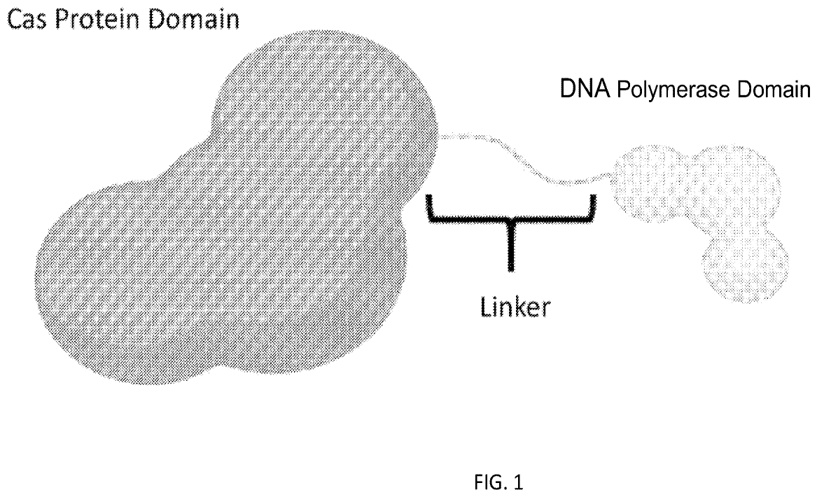

FIG. 2 is a graph showing prime editing at the HEK3 site in HEK293T cells using a SluCas9 prime editor and various PEgRNAs.

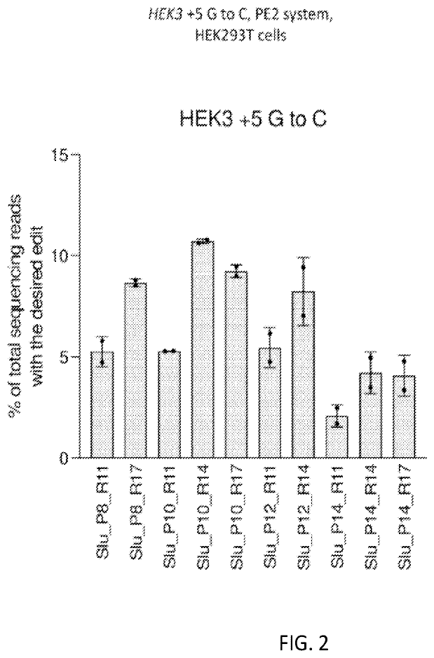

FIG. 3 is a graph showing prime editing at the FANCF locus in HEK293T cells using a prime editor with a DNA binding domain that is SpCas9, and a prime editor with a DNA binding domain that is SluCas9 with various PEgRNAs.

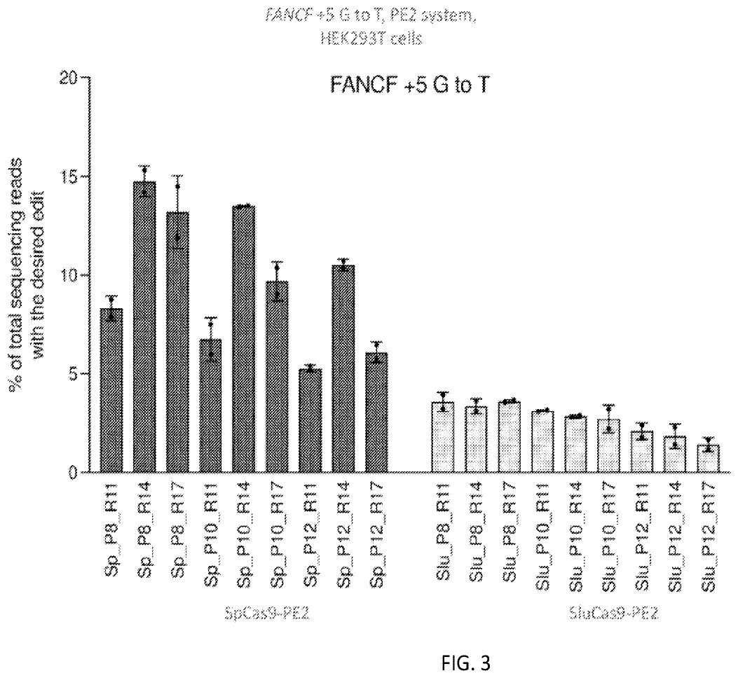

FIG. 4 is a graph showing editing at the FANCF locus in HEK293T cells using a prime editor with a SluCas9 DNA binding domain (left bar of pair) or a sRGN 3.3 Cas9 DNA binding domain (right bar of pair) with various PEgRNAs.

FIG. 5 is a graph showing percent editing at the VEGFA locus in HEK293 cells using prime editors with various RT homolog domains.

FIG. 6 shows illustrations of unstructured, structured, and natural linker variants useful in the prime editors disclosed herein.

FIG. 7 is a graph showing the average gene editing activity across 3 endogenous sites for prime editors comprising 47 linker variants in human HEK293T cells; the grey dot is PE2.

FIG. 8 is a graph showing the change in gene editing efficiency relative to PE2 at gene 6 endogenous sites in human HEK293T cells for prime editors comprising seven exemplary linker variants.

FIG. 9A is a maximum likelihood phylogenetic tree of RT homolog family. The scale bar represents 2 substitutions per site.

FIG. 9B is a phylogenetic tree with taxons that best represent the topology of the tree in FIG. 9A. Individual clades of RT homologs are labelled.

FIG. 10A is a simplified cartoon schematic of the domain structure of a prime editor (PE) with a Streptococcus Pyogenes Cas9 (SpCas9) domain and an RT homolog domain connected by a linker (N- and C-terminal nuclear localization signals not shown).

FIG. 10B is a box plot of prime editing efficiency at target loci VEGFA, RNF2, and HEK3 using prime editors with RT homolog sequences sampled from multiple RT homolog family members. The Y-axis indicates the percent (%) editing of the prime editor. Percent editing at three genomic loci are depicted by a dot colored according to the legend at the top. The X-axis lists the prime editors with different RT homolog sequences. The subfamily Glade of the RT homolog is labeled at the bottom. A canonical prime editor (PE) listed on the far-right is shown for comparison.

FIG. 11A is a maximum likelihood phylogenetic tree of the Zebrafish endogenous retrovirus (ZFERV) family of retroviral RTs. Reconstructed nodes in the tree, or inferred ancestral sequences, selected for gene synthesis and characterization are labeled with the corresponding node ID. The scale bar represents substitutions per site.

FIG. 11B contains bar plots showing prime editing efficiency at target loci VEGFA, RNF2, and HEK3 using PE containing inferred ancestral ZFERV RT sequences. The Y-axis indicates the percent (%) editing of the prime editor. The X-axis lists the prime editors with different ancestral ZFERV RT sequences. Prime editing efficiency using PE2 is included on the far-left of the plots for comparison.

FIG. 12A is a schematic of the construct layouts of PEs with different B1 domains of Streptococcal protein G (GB1 domains) and either full length or truncated Moloney Murine Leukemia Virus (MMLV) RT domains. The position of the amino acid sequence is labeled at the top.

FIG. 12B contains box plots of prime editing efficiency at target loci VEGFA, RNF2, and HEK3 using prime editors depicted in FIG. 12A. The Y-axis indicates the percent (%) editing of the prime editors. The X-axis lists the prime editors with GB1 domains and MMLV RT domains. The prime editing efficiency of a PE2 without a GB1 domain is shown for comparison.

FIG. 13A is a simplified cartoon schematic of the domain structure of an engineered Cas-RT prime editor where the Cas1 domain of a naturally occurring Cas1-RT fusion protein is replaced with a Cas9 domain. The CasRT and Cas9 domains are connected by the endogenouslinker. N- and C-terminal nuclear localization signals are typically included in the engineered Cas-RT prime editors (not shown).

FIG. 13B contains bar plots of prime editing efficiency at target loci VEGFA and RNF2 using different Cas-RT prime editors. The Y-axis indicates the percent (%) editing of the prime editors. The X-axis lists the Cas-RT prime editors. PE2 prime editing efficiency is shown for comparison.

FIG. 14 contains schematics for six different RT families. The domain comprising conserved sequences are illustrated on the top. The specific amino acid and sequence motif at each domain for various families are also shown. Sequences of conserved motifs, e.g., SEQ ID NOs. 905-909 and 1101-1102, respectively, are indicated for each RT family in order of appearance.

### DETAILED DESCRIPTION OF THE DISCLOSURE

Provided herein, in some embodiments, are compositions and methods related to prime editors. In some embodiments, the prime editors (PEs) provided herein can use engineered guide polynucleotides, e.g., prime editing guide RNAs (PEgRNAs), that can direct PEs to specific DNA targets and can encode DNA edits on the target gene that serve a variety of functions, including correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” means within an acceptable error range for the particular value as determined by one of ordinary skill in the art, which will depend in part on how the value is measured or determined, i.e, the limitations of the measurement system. For example, “about” can mean within 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell can be of or derived from different tissues, organs, and/or cell types. In some embodiments, the cell is a primary cell. As used herein, the term “primary cell” means a cell isolated from an organism, e.g., a mammal, which is grown in tissue culture (i.e., in vitro) for the first time before subdivision and transfer to a subculture. In some embodiments, the cell is a stem cell. In some non-limiting examples, mammalian cells, including primary cells and stem cells, can be modified through introduction of one or more polynucleotides, polypeptides, and/or prime editing compositions (e.g., through transfection, transduction, electroporation, and the like) and further passaged. Such modified cells include muscle cells (e.g., cardiac muscle cells, smooth muscle cells, hepatocytes), hematopoietic stem cells (HSCs), hematopoietic stem progenitor cells (HSPC)s, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. In some embodiments, the cell is a stem cell. In some embodiments, the cell is a progenitor cell. In some embodiments, the cell is a pluripotent cell (e.g., a pluripotent stem cell) In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is an embryonic stem cell (ESC).

In some embodiments, the cell is a human stem cell. In some embodiments, the cell is a human pluripotent stem cell. In some embodiments, the cell is a human fibroblast. In some embodiments, the cell is an induced human pluripotent stem cell. In some embodiments, the cell is a human stem cell. In some embodiments, the cell is a human embryonic stem cell.

In some embodiments, the cell is a CD34⁺ cell. In some embodiments, the cell is a hematopoietic stem cell (HSC). In some embodiments, the cell is a hematopoietic progenitor cell (HPC). In some embodiments, hematopoietic stem cells and hematopoietic progenitor cells are referred to as hematopoietic stem or progenitor cells (HSPCs). In some embodiments, the cell is a human HSC. In some embodiments, the cell is a human HPC. In some embodiments, the cell is a human HSPC. In some embodiments, the cell is a long term (LT)-HSC. In some embodiments, the cell is a short-term (ST)-HSC. In some embodiments, the cell is a myeloid progenitor cell. In some embodiments, the cell is a lymphoid progenitor cell. In some embodiments, the cell is a granulocyte monocyte progenitor cell. In some embodiments, the cell is a megakaryocyte erythroid progenitor cell. In some embodiments, the cell is a multipotent progenitor cell (MPP).

In some embodiments, the cell is a stem cell. In some embodiments, the cell is a human stem cell. In some embodiments, the cell is a hematopoietic stem cell (HSC) or a hematopoietic stem and progenitor cell. In some embodiments, the HSC is from bone marrow or mobilized peripheral blood. In some embodiments the human stem cell is an induced pluripotent stem cell (iPSC). In some embodiments, the cell is a human HSC. In some embodiments, the cell is a human CD34+ cell. In some embodiments, the cell is a hematopoietic stem and progenitor cell (HSPC). In some embodiments, the cell is a human hematopoietic stem and progenitor cell (HSPC). In some embodiments, the cell is a hematopoietic progenitor cell, multipotent progenitor cell, lymphoid progenitor cell, a myeloid progenitor cell, a megakaryocyte-erythroid progenitor cell, a granulocyte-megakaryocyte progenitor cell, a granulocyte, a promyelocyte, a neutrophil, an eosinophil, a basophil, an erythrocyte, a reticulocyte, a thrombocyte, a megakaryoblast, a platelet-producing megakaryocyte, a monocyte, a macrophage, a dendritic cell, a microglia, an osteoclast, a lymphocyte, a NK cell, a B-cell, or a T-cell. In some embodiments, the cell edited by prime editing can be differentiated into, or give rise to recovery of a population of cells, e.g., common lymphoid progenitor cells, common myeloid progenitor cells, megakaryocyte-erythroid progenitor cells, granulocyte-megakaryocyte progenitor cells, granulocytes, promyelocytes, neutrophils, eosinophils, basophils, erythrocytes, reticulocytes, thrombocytes, megakaryoblasts, platelet-producing megakaryocytes, platelets, monocytes, macrophages, dendritic cells, microglia, osteoclasts, lymphocytes, such as NK cells, B-cells or T-cells. In some embodiments, the cell edited by prime editing can be differentiated into, or give rise to recovery of a population of cells, e.g., neutrophils, platelets, red blood cells, monocytes, macrophages, antigen-presenting cells, microglia, osteoclasts, dendritic cells, inner ear cell, inner ear support cell, cochlear cell and/or lymphocytes. In some embodiments, the cell is in a subject, e.g., a human subject.

In some embodiments, a cell is not isolated from an organism but forms part of a tissue or organ of an organism, e.g., a mammal. In some non-limiting examples, mammalian cells include formed elements of the blood (e.g., lymphocytes, bone marrow cells), precursors of any of these somatic cell types, and stem cells.

In some embodiments, a cell is isolated from an organism. In some embodiments, a cell is derived from an organism. In some embodiments, a cell is a differentiated cell. In some embodiments, the cell is a fibroblast. In some embodiments, the cell is differentiated from an induced pluripotent stem cell. In some embodiments, the cell is differentiated from an HSC or an HPSC. In some embodiments, the cell is differentiated from an induced pluripotent stem cell (iPSC). In some embodiments, the cell is differentiated from an embryonic stem cell (ESC).

In some embodiments, the cell is a differentiated human cell. In some embodiments, cell is a human fibroblast. In some embodiments, the cell is differentiated from an induced human pluripotent stem cell. In some embodiments, the cell is differentiated from a human iPSC or a human ESC.

In some embodiments, the cell comprises a prime editor disclosed herein. In some embodiments, the cell comprises a prime editor, a PEgRNA, or a prime editing composition disclosed herein. In some embodiments, the cell is from a human subject. In some embodiments, the human subject has a disease or condition, or is at a risk of developing a disease or a condition associated with a mutation to be corrected by prime editing. In some embodiments, the cell comprises a mutation associated with a disease or disorder. In some embodiments, the cell is from a human subject, and comprises a prime editor, a PEgRNA, or a prime editing composition disclosed herein for correction of the mutation. In some embodiments, the cell is from the human subject, and the mutation has been edited or corrected by prime editing. In some embodiments, the cell is in a human subject. In some embodiments, the cell comprises a prime editor or a prime editing composition for correction of the mutation. In some embodiments, the cell is in a human subject, and comprises a prime editor or a prime editing composition for correction of the mutation. In some embodiments, the mutation in the cell has been edited or corrected by prime editing. In some embodiments, the cell is from the human subject and the mutation has been edited or corrected by prime editing.

The term “substantially” as used herein can refer to a value approaching 100% of a given value. In some embodiments, the term can refer to an amount that can be at least about 70%, 80%, 90%, 91%, 92%, 93%, 94%, 95%, 96%, 97%, 98%, 99%, 99.9%, or 99.99% of a total amount. In some embodiments, the term can refer to an amount that can be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 ammo acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein can be a full-length protein (e.g., a fully processed protein having certain biological function). In some embodiments, a protein can be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor can be a fusion protein comprising a Cas9 protein domain of S. pyogenes and a reverse transcriptase protein domain of a retrovirus (e.g., Moloney murine leukemia virus) or a variant of the retrovirus. A protein that comprises amino acid sequences from different origins or naturally occurring proteins can be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase can encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof can retain one or more of the functions of at least one of the functional domains. For example, a functional fragment of a Cas9 can encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase can comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof can retain one or more of the functions of at least one of the functional domains. For example, in some embodiments, a functional fragment of a Cas9 can comprise one or more amino acid substitutions in a nuclease domain, e.g., a H840A amino acid substitution, compared to the amino acid sequence of a wild type Cas9, but retains the DNA binding ability and lacks the nuclease activity partially or completely.

The term “function” and its grammatical equivalents as used herein refer to a capability of operating, having, or serving an intended purpose. Functional can comprise any percent from baseline to 100% of an intended purpose. For example, functional can comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional can mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence, or a polynucleotide sequence and a corresponding reference polynucleotide sequence. “Homology” can refer to polymeric sequences, e.g., polypeptide or DNA sequences that are similar Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences can exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or a protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs can also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions can be determined in homologous sequences based on alignment, for example, “H840” in a reference SpCas9 sequence can correspond to H839 where a variant SpCas9 sequence omits the N-terminal Methionine, or another corresponding position in a Cas9 homolog when the Cas9 homolog is aligned against the reference SpCas9 sequence.

The term “homolog” as used herein refers to a gene or a protein that is related to another gene or protein by a common ancestral DNA sequence. A homolog can be an ortholog or a paralog. An ortholog refers to a gene or protein that is related to another gene or protein by a speciation event. A paralog refers to a gene or protein that is related to another gene or protein by a duplication event within a genome. A paralog may be within the same species of the gene or protein it is related to. A paralog may also be in a different species of the gene or protein it is related to. In some embodiments, an ortholog may retain the same function. In some embodiments, a paralog may evolve a new function.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double-stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide can comprise one or more other nucleotide bases, such as inosine (I), which is read by the translation machinery as guanine (G).

In some embodiments, a polynucleotide can be modified. As used herein, the terms “modified” or “modification” refers to chemical modification with respect to the A, C, G, T and U nucleotides. In some embodiments, modifications can be on the nucleoside base and/or sugar portion of the nucleosides that comprise the polynucleotide. In some embodiments, the modification can be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides can base pair via hydrogen bonding, which can be Watson Crick, Hoogsteen or reversed Hoogsteen hydrogen bonding. For example, an adenine on one polynucleotide molecule will base pair to a thymine or uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity can be a region of 10, 15, 20, 25, 30, 35, 40, 45, 50, or more nucleotides. “Substantial complementary” can also refer to a 100% complementarity over a portion or region of two polynucleotide molecules. In some embodiments, the portion or region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression can include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The term “sequencing” as used herein, can comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or a nucleic acid sequence of a polynucleotide. Such changes and/or alterations can comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein can refer to a human or a non-human. A subject can be a mammal. A human subject can be male or female. A human subject can be of any age. A subject can be a human embryo. A human subject can be a newborn, an infant, a child, an adolescent, or an adult. A human subject can be up to about 100 years of age. A human subject can be in need of treatment for a genetic disease or disorder.

The terms “treatment” or “treating” and their grammatical equivalents refer to the medical management of a subject with an intent to cure, ameliorate, or ameliorate a symptom of, a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include supportive treatment, that is, treatment employed to supplement another specific therapy directed toward the improvement of the disease, condition, or disorder. In some embodiments, a condition can be pathological. In some embodiments, a treatment may not completely cure or prevent a disease, condition, or disorder. In some embodiments, a treatment ameliorates a disease, condition, or disorder. In some embodiments, a subject can be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g. a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example, a prime editing composition comprising a construct, that can be sufficient to result in a desired activity upon introduction into a subject as disclosed herein. An effective amount of the prime editing compositions can be provided to the target gene or cell, whether the cell is ex vivo or in vivo. An effective amount can be the amount to induce, for example, at least about a 2-fold change (increase or decrease) or more in the amount of target nucleic acid modulation (e.g., expression of a gene to produce functional a protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target gene to produce a functional protein).

The amount of target gene modulation can be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

An effective amount can be the amount to induce, when administered to a population of cells, a certain percentage of the population of cells to have a correction a mutation. For example, in some embodiments, an effective amount can be the amount to induce, when administered to or introduced to a population of cells, installation of one or more intended nucleotide edits that correct a mutation in the target gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

The term “reverse transcriptase” or “RT” as used herein refers to a class of enzymes that synthesize a DNA molecule from an RNA template. An RT may require the primer molecule with an exposed 3′ hydroxyl group. In some embodiments, the primer molecule of an RT may be a DNA molecule. In other cases, the primer molecule of an RT may be an RNA molecule. In some embodiments, an RT may comprise both DNA polymerase activity and RNase H activity. The two activities may reside in two separate domains in an RT.

The term “linker” as used herein refers to a bond, a chemical group, or a molecule linking two molecules or moieties, e.g., two p domains to form a fusion protein. A linker can be a peptide linker. A linker can also be a polynucleotide or oligonucleotide linker. For example, a RNA-binding protein recruitment sequence, such as a MS2 polynucleotide sequence, can be used to connect a Cas9 domain and a DNA polymerase domain of a prime editor, wherein one of the Cas9 domain and the DNA polymerase domain is fused to a MS2 coat protein. In some embodiments, a peptide linker may have various lengths, depending on the application of a linker or the sequences or molecules being linked by a linker.

The term “solubility-enhancement domain” or “SET domain” as used herein refers to a group of protein or peptide domains that enhance the solubility of a second protein or polypeptide when expressed as a fusion protein or polypeptide, relative to the second protein or polypeptide when expressed alone. A SET domain may also increase the activity of the second protein or polypeptide (e.g., enzymatic activity or nucleic acid-/protein-binding activity) when expressed as a fusion protein or polypeptide, relative to the second protein or polypeptide when expressed alone. A SET domain may also increase the expression level of the second protein or polypeptide when expressed as a fusion protein or polypeptide, relative to the second protein or polypeptide when expressed alone. A SET domain may also increase degree of folding to a native fold of the second protein or polypeptide when expressed as a fusion protein or polypeptide, relative to the second protein or polypeptide when expressed alone.

The term “fusion protein” refers to a protein comprised of domains from more than one naturally occurring or recombinantly produced protein, where generally each domain serves a different function. A domain may comprise a particular makeup of amino acids. A domain may also comprise a structure of proteins as described herein.

Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target gene of prime editing can comprise a double stranded DNA molecule having two complementary strands: a first strand that can be referred to as a “target strand” or a “non-edit strand”, and a second strand that can be referred to as a “non-target strand,” or an “edit strand.” In some embodiments, in a prime editing guide RNA (PEgRNA), a spacer sequence is complementary or substantially complementary to a specific sequence on the target strand, which can be referred to as a “search target sequence”. In some embodiments, the spacer sequence anneals with the target strand at the search target sequence. The target strand can also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence can have a substantially identical sequence as the protospacer sequence on the edit strand of a target gene, except that the spacer sequence can comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is 3 base pairs upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

A “primer binding site” (PBS or primer binding site sequence) is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e., the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

In some instances, a prime editor of this disclosure is configured to bind a prime editing guide RNA (PEgRNA). In some cases, a PEgRNA comprises at least one of: a spacer, an extension arm, and a gRNA core. In some cases, a spacer may comprise a sequence that hybridizes to a first strand of a double stranded target DNA sequence. In some cases, a spacer may comprise a sequence that is complementary to a first strand of a double stranded target DNA sequence. For example, the spacer may comprise complementary sequence to a protospacer sequence in the first strand of the double stranded DNA sequence. In some cases, an extension arm may comprise a sequence that hybridizes to a second strand (i.e., the complementary strand of the first strand) of the double stranded target DNA sequence. In some cases, an extension arm may comprise a sequence that is complementary to a second strand (i.e., the complementary strand of the first strand) of the double stranded target DNA sequence. In some cases, a gRNA core may comprise a sequence that interacts with the second polypeptide (i.e., interacts with DNA binding domain of the PE). In some instances, a nucleotide of a PEgRNA may be part of a spacer. In some cases, a nucleotide of a PEgRNA may be part of an extension arm. In some cases, a nucleotide of a PEgRNA may be part of a gRNA core. In some cases, a nucleotide of a PEgRNA may be part of a spacer and an extension arm. In some cases, a nucleotide of a PEgRNA may be part of a spacer and a gRNA core. In some cases, a nucleotide of a PEgRNA may be part of an extension arm and a gRNA core. In some cases, a nucleotide of a PEgRNA may be part of a spacer and an extension arm. In some cases, a nucleotide of a PEgRNA may not be part of a spacer, an extension arm, or a gRNA core.

In some instances, a PEgRNA may be transcribed as a single RNA sequence. In some cases, a spacer, an extension arm, and a gRNA core may be in a single stranded RNA sequence. In some cases, a spacer, an extension arm, and a gRNA core may be in a single strand of a double stranded RNA sequence. In some cases, a PEgRNA may comprise a spacer, an extension arm, and a gRNA core in a single RNA sequence. In some cases, a PEgRNA may comprise a spacer, an extension arm, and a gRNA core in a single RNA sequence in a 5′-3′ orientation. In some cases, a PEgRNA may comprise a gRNA core, an extension arm, and a spacer in a single RNA sequence in a 3′-5′ orientation. In some cases, a PEgRNA may comprise a spacer, a gRNA core, and an extension arm are in a single RNA sequence in a 5′-3′ orientation. In some cases, a PEgRNA may comprise an extension arm, a spacer, and a gRNA core are in a single RNA sequence in 5′-3′ orientation. In some cases, a PEgRNA may comprise an extension arm, a gRNA core, and a spacer in a single RNA sequence in 5′-3′ orientation. In some cases, a PEgRNA may comprise a gRNA core, an extension arm, and a spacer in a single RNA sequence in 5′-3′ orientation. In some cases, a PEgRNA may comprise a gRNA core, a spacer and an extension arm in a single RNA sequence in 5′-3′ orientation. In some instances, a PEgRNA may be transcribed as multiple RNA molecules. In some cases, a spacer, an extension arm, and a gRNA core may be in multiple single stranded RNA sequences. In some cases, a spacer may be in a single stranded RNA sequence. In some cases, an extension arm may be in a single stranded RNA sequence. In some cases, a gRNA core may be in a single stranded RNA sequence.

In some cases, a spacer may comprise a sequence that hybridizes to a first strand of a double stranded target DNA sequence. In some cases, a spacer may comprise a sequence that is complementary to a first strand of a double stranded target DNA sequence. In some cases, a spacer may hybridize to a first strand of a double stranded target DNA sequence through complementary base pairing of the nucleotides. In some cases, a spacer may hybridize to a protospacer of a first strand of a double stranded target DNA sequence. In some cases, a spacer may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In some instances, an extension arm of a PEgRNA may comprise a primer binding site (PBS). In some cases, a second strand of a double stranded target DNA sequence may bind to the PBS of a PEgRNA. In some cases, the PBS of a PEgRNA comprises a sequence that is complementary to a second strand of a double stranded target DNA sequence. In some cases, a second strand of a double stranded target DNA sequence may bind to the PBS of a PEgRNA after the second strand of the double stranded target DNA sequence is nicked or cleaved by a prime editor (e.g., any prime editor described). In some cases, the second strand of the double stranded target DNA sequence binding or bound to the PBS of the PEgRNA may comprise a free 3′ hydroxyl end. In some cases, the PBS of a PEgRNA comprises a sequence that is complementary to a region upstream of the nick on the second strand of a double stranded target DNA sequence. In some cases, the PBS of a PEgRNA may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In some instances, an extension arm of a PEgRNA may comprise a DNA synthesis template. In some cases, an extension arm of a PEgRNA may comprise a PBS or a DNA synthesis template. In other cases, an extension arm of a PEgRNA may comprise a PBS and a DNA synthesis template. In some instances, a DNA synthesis template of a PEgRNA may comprise a nucleotide edit, as compared to a double stranded target DNA sequence.

In some instances, the DNA synthesis template may comprise a portion that is homologous to the double stranded target DNA sequence. In some instances, the DNA synthesis template may be homologous to the double stranded target DNA sequence. The homology between the DNA synthesis template and the double stranded target DNA sequence, in some cases, may be at least about 85%, absent a nucleotide edit in the DNA synthesis template. The homology between the DNA synthesis template and the double stranded target DNA sequence, in some cases, may be at least about 90%, absent a nucleotide edit in the DNA synthesis template. The homology between the DNA synthesis template and the double stranded target DNA sequence, in some cases, may be at least about 95%, absent a nucleotide edit in the DNA synthesis template. The homology between the DNA synthesis template and the double stranded target DNA sequence, in some cases, may be at least about 96%, absent a nucleotide edit in the DNA synthesis template. The homology between the DNA synthesis template and the double stranded target DNA sequence, in some cases, may be at least about 97%, absent a nucleotide edit in the DNA synthesis template. The homology between the DNA synthesis template and the double stranded target DNA sequence, in some cases, may be at least about 98%, absent a nucleotide edit in the DNA synthesis template. The homology between the DNA synthesis template and the double stranded target DNA sequence, in some cases, may be at least about 99%, absent a nucleotide edit in the DNA synthesis template. The homology between the DNA synthesis template and the double stranded target DNA sequence, in some cases, may be at least about 99.9%, absent a nucleotide edit in the DNA synthesis template. The homology between the DNA synthesis template and the double stranded target DNA sequence, in some cases, may be at least about 50%, at least about 55%, at least about 60%, at least about 65%, at least about 70%, at least about 75%, or at least about 80%, absent a nucleotide edit in the DNA synthesis template. The homology between the DNA synthesis template and the double stranded target DNA sequence, in some cases, may be from about 50 to about 60%, from about 55 to about 65%, from about 60 to about 70%, from about 65 to about 75%, from about 70 to about 80%, from about 75 to about 85%, from about 80 to about 90%, from about 85 to about 95%, or from about 90 to about 100%, absent a nucleotide edit in the DNA synthesis template.

In some instances, the DNA synthesis template may be homologous to the double stranded target DNA sequence. The homology between the DNA synthesis template and the double stranded target DNA sequence, in some cases, may be at least about 85%, with a nucleotide edit in the DNA synthesis template. The homology between the DNA synthesis template and the double stranded target DNA sequence, in some cases, may be at least about 90%, with a nucleotide edit in the DNA synthesis template. The homology between the DNA synthesis template and the double stranded target DNA sequence, in some cases, may be at least about 95%, with a nucleotide edit in the DNA synthesis template. The homology between the DNA synthesis template and the double stranded target DNA sequence, in some cases, may be at least about 96%, with a nucleotide edit in the DNA synthesis template. The homology between the DNA synthesis template and the double stranded target DNA sequence, in some cases, may be at least about 97%, with a nucleotide edit in the DNA synthesis template. The homology between the DNA synthesis template and the double stranded target DNA sequence, in some cases, may be at least about 98%, with a nucleotide edit in the DNA synthesis template. The homology between the DNA synthesis template and the double stranded target DNA sequence, in some cases, may be at least about 99%, with a nucleotide edit in the DNA synthesis template. The homology between the DNA synthesis template and the double stranded target DNA sequence, in some cases, may be at least about 99.9%, with a nucleotide edit in the DNA synthesis template. The homology between the DNA synthesis template and the double stranded target DNA sequence, in some cases, may be at least about 50%, at least about 55%, at least about 60%, at least about 65%, at least about 70%, at least about 75%, or at least about 80%, with a nucleotide edit in the DNA synthesis template. The homology between the DNA synthesis template and the double stranded target DNA sequence, in some cases, may be from about 50 to about 60%, from about 55 to about 65%, from about 60 to about 70%, from about 65 to about 75%, from about 70 to about 80%, from about 75 to about 85%, from about 80 to about 90%, from about 85 to about 95%, or from about 90 to about 100%, with a nucleotide edit in the DNA synthesis template.

In some instances, the DNA synthesis template may comprise a nucleotide sequence comprising at least about 85% sequence identity to a strand of a target DNA sequence. In some cases, the DNA synthesis template may comprise a nucleotide sequence comprising at least about 90% sequence identity to a strand of a target DNA sequence. In some cases, the DNA synthesis template may comprise a nucleotide sequence comprising at least about 95% sequence identity to a strand of a target DNA sequence. In some cases, the DNA synthesis template may comprise a nucleotide sequence comprising at least about 96%, at least about 97%, at least about 98%, or at least about 99% sequence identity to a strand of a target DNA sequence. In some cases, the DNA synthesis template may comprise a nucleotide sequence comprising at least about 86%, at least about 87%, at least about 88%, or at least about 89% sequence identity to a strand of a target DNA sequence. In some cases, the DNA synthesis template may comprise a nucleotide sequence comprising at least about 50%, at least about 55%, at least about 60%, at least about 65%, at least about 70%, at least about 75%, or at least about 80% sequence identity to a strand of a target DNA sequence.

In some instances, the DNA synthesis template may comprise a nucleotide sequence comprising at least about 85% sequence identity to a first strand of a target DNA sequence. In some cases, the DNA synthesis template may comprise a nucleotide sequence comprising at least about 90% sequence identity to a first strand of a target DNA sequence. In some cases, the DNA synthesis template may comprise a nucleotide sequence comprising at least about 95% sequence identity to a first strand of a target DNA sequence. In some cases, the DNA synthesis template may comprise a nucleotide sequence comprising at least about 96%, at least about 97%, at least about 98%, or at least about 99% sequence identity to a first strand of a target DNA sequence. In some cases, the DNA synthesis template may comprise a nucleotide sequence comprising at least about 86%, at least about 87%, at least about 88%, or at least about 89% sequence identity to a first strand of a target DNA sequence. In some cases, the DNA synthesis template may comprise a nucleotide sequence comprising at least about 50%, at least about 55%, at least about 60%, at least about 65%, at least about 70%, at least about 75%, or at least about 80% sequence identity to a first strand of a target DNA sequence.

In some embodiments, a PEgRNA complexes with, and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site on the edit strand, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to an endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template can be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing. Prime editors described herein may comprise multiple polypeptides or protein domains. In various embodiments, a prime editor includes a polypeptide domain having DNA binding activity (e.g., a DNA binding domain). In some embodiments, a prime editor comprises a polypeptide that comprises a DNA binding domain. In some embodiments, a prime editor includes a polypeptide domain having DNA polymerase activity (e.g., a DNA polymerase domain). In some embodiments, a prime editor comprises a polypeptide that comprises a DNA polymerase domain. In various embodiments, a prime editor comprises a polypeptide domain having DNA binding activity (e.g., a DNA binding domain), and a polypeptide domain having DNA polymerase activity (e.g., a DNA polymerase domain) In some embodiments, a prime editor comprises a polypeptide that comprises a DNA binding domain and a polypeptide that comprises a DNA polymerase domain.

In some embodiments, the prime editor further comprises a polypeptide domain having a nuclease activity. In some embodiments, the polypeptide domain having the nuclease activity comprises a nickase, or a fully active nuclease. In some embodiments, the DNA binding domain comprises a nuclease domain or nuclease activity. In some embodiments, the nuclease domain is a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the DNA binding domain comprises a nuclease domain that is an inactive nuclease.

In some embodiments, the polypeptide domain having DNA binding activity (e.g., programmable DNA binding activity) comprises a nucleic acid guided DNA binding domain, for example, a CRISPR-Cas protein, for example, a Cas9 nickase, a Cpf1 nickase, or another CRISPR-Cas nuclease. In some embodiments, the DNA binding domain is a nucleic acid guided DNA binding domain for example, a CRISPR-Cas protein, for example, a Cas9 nickase, a Cpf1 nickase, or another CRISPR-Cas nuclease. In some embodiments, the DNA binding domain (e.g., a nucleic acid guided DNA binding domain is a Cas protein domain. In some embodiments, the Cas protein is a Cas9. In some embodiments, the Cas protein domain comprises a nickase or comprises a nickase activity.

In some embodiments, the polypeptide domain having DNA polymerase activity comprises a template-dependent DNA polymerase, for example, a DNA-dependent DNA polymerase or an RNA-dependent DNA polymerase. In some embodiments, the DNA binding domain comprises a template-dependent DNA polymerase for example, a DNA-dependent DNA polymerase or an RNA-dependent DNA polymerase. In some embodiments, the DNA polymerase domain comprises a reverse transcriptase domain (RT domain) or a reverse transcriptase (RT). In some embodiments, the DNA polymerase domain is a RT domain or a RT. In some embodiments, a prime editor comprises a reverse transcriptase (RT) activity. For example, the first polypeptide of the prime editor may have activity for target primed reverse transcription. In some embodiments, the polypeptide domain having DNA polymerase activity comprises a reverse transcriptase activity (e.g., activity for target primed reverse transcription).

In some embodiments, the prime editor comprises additional polypeptides involved in prime editing, for example, a polypeptide domain having a 5′ endonuclease activity, e.g., a 5′ endogenous DNA flap endonuclease (e.g., FEN1), for helping to drive the prime editing process towards the edited product formation. In some embodiments, the prime editor further comprises an RNA-protein recruitment polypeptide, for example, a MS2 coat protein.

In some embodiments, polypeptide domains of a prime editor (e.g., a DNA binding domain, a DNA polymerase domain) can be fused or linked by a peptide linker to form a fusion protein. In other embodiments, a prime editor comprises one or more polypeptide domains (e.g., a DNA binding domain, and a DNA polymerase domain) provided in trans as separate proteins, which are capable of being associated to each other through non-peptide linkages or through aptamers or recruitment sequences. For example, a prime editor can comprise a DNA binding domain and a DNA polymerase domain (e.g., a reverse transcriptase domain) fused or linked with each other by a peptide linker (e.g., linkers disclosed set forth in SEQ ID NOs: 273-318). For example, a prime editor can comprise a DNA binding domain and a DNA polymerase domain (e.g., a reverse transcriptase domain) associated with each other by an RNA-protein recruitment aptamer, e.g., a MS2 aptamer, which can, in some embodiments, be linked to a PEgRNA. Prime editor polypeptide components can be encoded by one or more polynucleotides in whole or in part. The present disclosure contemplates polynucleotides encoding the prime editor components, for example, a polynucleotide encoding a DNA binding domain, and a polynucleotide encoding a DNA polymerase domain. The present disclosure also contemplates a single polynucleotide comprising a polynucleotide encoding a DNA binding domain, and a polynucleotide encoding a DNA polymerase domain. In some embodiments, the polynucleotide encoding a DNA binding domain, and a polynucleotide encoding a DNA polymerase domain are linked by a linker polynucleotide to result in a fusion protein comprising the DNA polymerase domain and DNA binding domain linked by a linker. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein can comprise an N-terminal portion fused to an intein-N and a C-terminal portion fused to an intein-C, each of which is individually encoded by an AAV vector. In some embodiments, components of a prime editor disclosed herein (e.g., a polypeptide comprising a DNA binding domain and/or a polypeptide comprising a DNA polymerase domain) may be brought together post-translationally via a split-intein.

In some embodiments, a prime editor comprises a DNA polymerase domain and a DNA binding domain wherein the amino acid sequences of the DNA polymerase domain and the DNA binding domain comprise a N terminus methionine. In some embodiments, a prime editor comprises a DNA polymerase domain and a DNA binding domain wherein the amino acid sequences of the DNA polymerase domain and the DNA binding domain do not comprise a N terminus methionine. In some embodiments, a prime editor comprises a DNA polymerase domain and a DNA binding domain wherein the amino acid sequence of the DNA polymerase domain comprises a N terminus methionine and the amino acid sequence of the DNA binding domain does not comprise a N terminus methionine. In some embodiments, a prime editor comprises a DNA polymerase domain and a DNA binding domain wherein the amino acid sequence of the DNA polymerase domain does not comprise a N terminus methionine and the amino acid sequence of the DNA binding domain comprises a N terminus methionine

A prime editor component thereof (e.g., a polypeptide comprising a DNA binding domain and/or a polypeptide comprising a DNA polymerase domain) can be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor can comprise a S. pyogenes Cas9 polypeptide and a Moloney murine leukemia virus (M-MLV) reverse transcriptase polypeptide.

An RT or an RT domain may be rationally engineered, in some embodiments. Such an engineered RT or RT domain may comprise sequences or amino acid changes different from a naturally occurring RT or RT domain. In some embodiments, the engineered RT or RT domain may have improved RT activity over a naturally occurring RT or RT domain. In some embodiments, the engineered RT or RT domain may have improved prime editing efficiency over a naturally occurring RT or RT domain, when used in a prime editor.

In some embodiments, the prime editor comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NO: 125-128, 504-521, 939-987, or 1007-1013, (Tables 2, 4A, 6, 8, 10, 14, 15, and 16). In some embodiments, the prime editor comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences listed in Tables 2, 4A, 6, 8, 10, 14, 15, and/or 16. In some embodiments, the prime editor comprises an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the ammo acid sequences set forth in SEQ ID NO: 125-128, 504-521, 939-987, or 1007-1013. In some embodiments, the prime editor comprises an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the amino acid sequences listed in any one of the Tables 2, 4A, 6, 8, 10, 14, 15, and/or 16. In some embodiments, the prime editor comprises an amino acid sequence identical to any one of the sequences set forth in SEQ ID NO: 125-128, 504-521, 939-987, or 1007-1013 (Tables 2, 4A, 8, 10, 15, and 16). In some embodiments, the prime editor comprises an amino acid sequence identical to any one of the sequences listed in any of the tables 2, 4A, 8, 10, 15, and/or 16.

### DNA Polymerase Domain

In some embodiments, a prime editor comprises a polypeptide domain (e.g., a DNA polymerase domain) comprising a DNA polymerase activity. In some embodiments, the prime editor comprises a polypeptide that comprises a DNA polymerase domain. In some embodiments, a prime editor comprises a polynucleotide that encodes a polymerase domain, e.g., a DNA polymerase domain. The DNA polymerase domain can be a wild-type DNA polymerase domain, a full-length DNA polymerase protein domain, a wild type DNA polymerase, a full-length DNA polymerase, or can be a functional mutant, a functional variant, or a functional fragment thereof. In some embodiments, the DNA polymerase domain is a template dependent DNA polymerase domain. For example, the DNA polymerase can rely on a template polynucleotide strand, e.g., the editing template sequence, for new strand DNA synthesis. In some embodiments, the prime editor comprises a DNA-dependent DNA polymerase. In some embodiments, the DNA polymerase domain is a DNA-dependent DNA polymerase. For example, a prime editor having a DNA-dependent DNA polymerase can synthesize a new single stranded DNA using a PEgRNA editing template that comprises a DNA sequence as a template. In such embodiments, the PEgRNA can be a chimeric or hybrid PEgRNA, and comprising an extension arm comprising a DNA strand. The chimeric or hybrid PEgRNA can comprise an RNA portion (including the spacer and the gRNA core) and a DNA portion (the extension arm comprising the editing template that includes a strand of DNA). In some embodiments, the prime editors provided herein comprises a DNA polymerase domain comprising an amino acid sequence that does not a have a N-terminus methionine. In some embodiments, the prime editors provided herein comprises a DNA polymerase domain comprising an amino acid sequence comprising a N-terminus methionine. In some embodiments, the amino acid sequence of a DNA polymerase domain may be N-terminally modified by one or more processing enzymes, e.g., by Methionine aminopeptidases (MAP).

The DNA polymerase domain can be a wild type DNA polymerase, for example, from eukaryotic, prokaryotic, archaeal, or viral organisms, and/or the polymerases may be modified by genetic engineering, mutagenesis, or directed evolution-based processes. The DNA polymerases can be a T7 DNA polymerase, T5 DNA polymerase, T4 DNA polymerase, Klenow fragment DNA polymerase, DNA polymerase III and the like. The DNA polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof. In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises an eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev 1 DNA polymerase. In some embodiments, the DNA polymerase is a human Revl DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

RT Homologs and Engineered RTs

In some embodiments, a prime editor comprises an RNA-dependent DNA polymerase domain, for example, a reverse transcriptase (RT). In some embodiments, the DNA polymerase domain is an RNA-dependent DNA polymerase domain, for example, a reverse transcriptase (RT). In some embodiments, the DNA polymerase domain is a reverse transcription (RT) domain, for example, a reverse transcriptase (RT). A RT or an RT domain can be a wild type RT domain, a full-length RT domain, or may be a functional mutant, a functional variant, or a functional fragment thereof. An RT or an RT domain of a prime editor may comprise a wild-type RT, a full length RT, a functional mutant, a functional variant, or a functional fragment thereof; or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT. In some embodiments, a prime editor comprising the engineered RT has improved prime editing efficiency over a prime editor having a PE2 RT reference.

In some embodiments, the reverse transcriptase domain or RT may be between 200 and 800 amino acids in length, between 300 and 700 amino acids in length, or at least 400 and 600 amino acids in length. The reverse transcriptase domain or RT may be at least 200 amino acids in length, at least 300 amino acids in length, at least 400 amino acids in length, at least 500 amino acids in length, or at least 600 amino acids in length. In some embodiments, the reverse transcriptase domain or RT is 250 amino acids in length. In some embodiments, the reverse transcriptase domain or RT is 350 amino acids in length. In some embodiments, the reverse transcriptase domain or RT is 450 amino acids in length. In some embodiments, the reverse transcriptase domain or RT is 550 amino acids in length. In some embodiments, the reverse transcriptase domain or RT is 650 amino acids in length.

In some embodiments, a prime editor comprises a virus RT, for example, a retrovirus RT. In some embodiments, the RT is a virus RT, for example, a retrovirus RT. Non-limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT. In some embodiments, the RT domain or RT is a wild type M-MLV RT. An exemplary sequence of a wild type M-MLV RT is provided in SEQ ID NO:857. In some embodiments, the prime editor comprises a reference M-MLV RT. In some embodiments, a MMLV RT, e.g., reference MMLV RT, comprises a sequence as disclosed in SEQ ID no: 855.

Exemplary reference moloney murine leukemia virus reverse transcriptase:

(SEQ ID NO: 855)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLIENSSP

In some embodiments, the prime editor comprises a M-MLV RT that comprises one or more of amino acid substitutions P51X, 567X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to a reference M-MMLV RT as set forth in SEQ ID NO:855, where X is any amino acid other than the reference amino acid. In some embodiments, the prime editor comprises a M-MLV RT that comprises one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO:855. In some embodiments, prime editor comprises one or more amino acid substitutions D200N, T330P, L603W, T306K, and W313F as compared to the reference M-MLV RT as set forth in SEQ ID NO: 855. In some embodiments, the prime editor comprises a M-MLV RT that comprises amino acid substitutions D200N, T330P, L603W, T306K, and W313F as compared to the reference M-MLV RT as set forth in SEQ ID NO:855. In some embodiments, the prime editor comprises a M-MLV RT as set forth in SEQ ID NO: 857. In some embodiments, the prime editor comprises a M-MLV RT as set forth in SEQ ID NO: 856. In some embodiments, the prime editor comprises a M-MLV RT as set forth in SEQ ID NO: 855. In some embodiments, the prime editor comprises a M-MLV RT as set forth in SEQ ID NO:884.

In some embodiments, the RT is a M-MLV RT that comprises one or more of amino acid substitutions P51X, 567X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 855, where X is any amino acid other than the wild type amino acid. In some embodiments, the RT is a M-MMLV RT that comprises one or more of amino acid substitutions P51L, S67K, E69K, L139P, T197A, D200N, H204R, F209N, E302K, E302R, T306K, F309N, W313F, T330P, L345G, L435G, P448A, D449G, N454K, D524G, E562Q, D583N, H594Q, L603W, E607K, and D653N as compared to the reference M-MMLV RT as set forth in SEQ ID NO: 855. In some embodiments, the RT is a M-MLV RT that comprises one or more amino acid substitutions D200N, T330P, L603W, T306K, and W313F as compared to the reference M-MLV RT as set forth in SEQ ID NO: 855. In some embodiments, the RT is a M-MLV RT that comprises amino acid substitutions D200N, T330P, L603W, T306K, and W313F as compared to the reference M-MLV RT as set forth in SEQ ID NO: 855. In some embodiments, the RT that is a M-MLVRT comprising the D200N, T330P, L603W, T306K, and W313F as compared to the reference M-MMLV RT (SEQ ID NO: 855) may be referred to as a “PE2” prime editor, and the corresponding prime editing system a PE2 prime editing system.

In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NOs: 1-95, 129-136, 198-271, 319-493, 533-846, 855-857, 884, or 990-1006. In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences listed in any of the Tables 1, 2, 3, 7, 14, 15, 16, or 23. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the amino acid sequences set forth in SEQ ID NOs: 1-95, 129-136, 198-271, 319-493, 533-846, 855-857, 884, or 990-1006. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any one of the amino acid sequences listed in any of the Tables 1, 2, 3, 7, 14, 15, 16, or 23. In some embodiments, a prime editor may comprise a RT domain having an amino acid sequence identical to any one of the sequences set forth in SEQ ID NO: 1-95, 129-136, 198-271, 319-493, 533-846, 855-857, 884, or 990-1006. In some embodiments, a prime editor may comprise a RT domain having an amino acid sequence identical to any one of the sequences listed in in any of the Tables 1, 2, 3, 7, 14, 15, 16, or 23.

In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NOs: 1-95, 198-271, 319-493, 855-857, 884, or 990-1006. In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NO: 5, 6, 13, 15, 16, 17, 18, 21, 22, 204, 230, 232-244, 249-257, 261, 270, 271, 327, 329, 332, 333, 337, 340, 341, 342, 344, 489, 990-1006, 209, 210, 231, 229. In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences listed in any of the Tables 1, 2, 15, or 16. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the amino acid sequences set forth in SEQ ID NOs: 1-95, 198-271, 319-493, 855-857, 884, or 990-1006. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the amino acid sequences set forth in SEQ ID NOs. 5, 6, 13, 15, 16, 17, 18, 21, 22, 204, 230, 232-244, 249-257, 261, 270, 271, 327, 329, 332, 333, 337, 340, 341, 342, 344, 489, 990-1006, 209, 210, 231, 229. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any one of the amino acid sequences listed in any of the Tables 1, 2, 15, or 16. In some embodiments, a prime editor may comprise a RT domain having an amino acid sequence identical to any one of the sequences set forth in SEQ ID NO: 1-95, 198-271, 319-493, 855-857, 884, or 990-1006. In some embodiments, a prime editor may comprise a RT domain having an amino acid sequence identical to any one of the sequences set forth in SEQ ID NO:5, 6, 13, 15, 16, 17, 18, 21, 22, 130, 131, 204, 230, 232-244, 249-257, 261, 270, 271, 327, 329, 332, 333, 337, 340, 341, 342, 344, 489, 990-1006, 209, 210, 231, 2291n some embodiments, a prime editor may comprise a RT domain having an amino acid sequence identical to any one of the sequences listed in in any of the Tables 1, 2, 15, or 16.

In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequences set forth in SEQ ID NO: 16. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to the amino acid sequences set forth in SEQ ID NO: 16. In some embodiments, a prime editor may comprise a RT domain having an amino acid sequence identical to the sequences set forth in SEQ ID NO: 16.

In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequences set forth in SEQ ID NO: 18. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to the amino acid sequences set forth in SEQ ID NO: 18. In some embodiments, a prime editor may comprise a RT domain having an amino acid sequence identical to the sequences set forth in SEQ ID NO: 18.

In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequences set forth in SEQ ID NO: 261. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to the amino acid sequences set forth in SEQ ID NO: 261. In some embodiments, a prime editor may comprise a RT domain having an amino acid sequence identical to the sequences set forth in SEQ ID NO: 261.

In some embodiments, a prime editor may comprise a RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequences set forth in SEQ ID NO: 270. In some embodiments, a prime editor may comprise a RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to the amino acid sequences set forth in SEQ ID NO: 270. In some embodiments, a prime editor may comprise a RT domain having an amino acid sequence identical to the sequences set forth in SEQ ID NO: 270.

In some embodiments, a RT domain may comprise an ancestral RT sequence. In some embodiments, a prime editor may comprise a RT domain having an amino acid sequence, e.g., an ancestral RT sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NO: 81-95. In some embodiments, a RT domain may comprise an ancestral RT sequence. In some embodiments, a prime editor may comprise a RT domain having an amino acid sequence, e.g., an ancestral RT sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NO: 81, 82, 84, 91. In some embodiments, a prime editor may comprise a RT domain having an amino acid sequence, e.g., an ancestral RT sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences listed in Table 3. In some embodiments, a prime editor may comprise a RT domain having an amino acid sequence, e.g., an ancestral RT sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e g, amino acid deletions, amino acid insertions, and/or amino acid substitutions compared to any of the amino acid sequences set forth in SEQ ID NO: 81-95. In some embodiments, a prime editor may comprise a RT domain having an amino acid sequence, e.g., an ancestral RT sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the amino acid sequences set forth in SEQ ID NO: 81, 82, 84, 91. In some embodiments, a prime editor may comprise a RT domain, having an amino acid sequence, e.g., an ancestral RT sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e g, amino acid deletions, amino acid insertions, and/or amino acid substitutions compared to any one of the amino acid sequences listed in Table 3. In some embodiments, a prime editor may comprise a RT domain, having an amino acid sequence, e.g., an ancestral RT sequence identical to any one of the sequences set forth in SEQ ID NO: 81-95. In some embodiments, a prime editor may comprise a RT domain, having an amino acid sequence, e.g., an ancestral RT sequence identical to any one of the sequences set forth in SEQ ID NO: 81, 82, 84, 91. In some embodiments, a prime editor may comprise a RT domain having an amino acid sequence, e.g., an ancestral RT sequence identical to any one of the sequences listed in Table 3.

In some embodiments, a prime editor may comprise a RT domain that is a Cas-RT. In some embodiments, the RT domains works with Cas1, Cas6, or Cas3 in RNA spacer acquisition. In some embodiments, a prime editor may comprise a RT domain, e.g., Cas-RT domain. In some embodiments, both Cas1 domain of Cas1-RT-Cas1 may be replaced with a Cas9 domain and optionally a linker sequence. In some embodiments, a prime editor may comprise a RT domain, e.g., a Cas-RT domain having an amino acid sequence, with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NO: 129-136, 345, 368, 396, or 533-846. In some embodiments, a prime editor may comprise a RT domain e.g., a Cas-RT domain having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences listed in Tables 1, 7, or 14. In some embodiments, a prime editor may comprise a RT domain, e.g., a Cas-RT domain having an amino acid sequence, that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., amino acid deletions, amino acid insertions, and/or amino acid substitutions compared to any of the amino acid sequences set forth in SEQ ID NO: 129-136, 345, 368, 396, or 533-846. In some embodiments, a prime editor may comprise a RT domain, e.g., a Cas-RT domain having an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences, e.g., mutations e.g., amino acid deletions, amino acid insertions, and/or amino acid substitutions compared to any one of the amino acid sequences listed in any of the Tables 1, 7, and/or 14. In some embodiments, a prime editor may comprise a RT domain, e.g., a Cas-RT domain, having an amino acid sequence identical to any one of the sequences set forth in SEQ ID NO: 129-136, 345, 368, 396, 533-846. In some embodiments, a prime editor may comprise a RT domain having an amino acid sequence, e.g., ancestral RT sequence identical to any one of the sequences listed in any of the Tables 1, 7, and/or 14. In some embodiments, a prime editor may comprise a RT domain that is Cas9-RT-Cas9 domain. In some embodiments, a Cas9-RT-Cas9 domain may further comprise a linker sequence.

In some embodiments, a DNA polymerase domain, e.g., a reverse transcriptase domain may comprise one or more mutations. Mutant reverse transcriptases can, for example, be obtained by mutating the gene or genes encoding the reverse transcriptase of interest by site-directed or random mutagenesis. In some embodiments, the mutation may include a deletion mutation, a point mutation, a substitutional mutation and/or an insertional mutation. In some embodiments, the mutation increases the efficiency of the DNA polymerase domain, e.g., a reverse transcriptase domain, e.g., by increasing editing efficiency, e.g., by increasing reverse transcriptase activity, e.g., by increasing stability (e.g., thermostability). In some embodiments, the mutated DNA polymerase domain, e.g., the mutated RT domain may show at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to an unmutated DNA polymerase domain, e.g., RT domain. In some embodiments, the mutated DNA polymerase domain, e.g., the mutated RT domain may show at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increased activity compared to an unmutated DNA polymerase domain, e.g., RT domain.

In some embodiments, a DNA polymerase domain, e.g., a RT domain may comprise one or more mutations selected from the group consisting of a P51 mutation, a S67 mutation, an E69 mutation, an L139 mutation, a T197 mutation, a D200 mutation, a H204 mutation, A F209 mutation, an E302 mutation, a T306 mutation, a F309 mutation, a W313 mutation, a T330 mutation, an L435 mutation, a P448 mutation, a D449 mutation, an N454 mutation, a D524 mutation, an E562 mutation, a D583 mutation, an H594 mutation, an L603 mutation, an E607 mutation, a G615 mutation, an H634 mutation, a G637 mutation, an H638 mutation, a D653 mutation, or an L671 mutation relative to the reference M-MLV RT as set forth in SEQ ID NO:855. In some embodiments, a DNA polymerase domain, e.g., a RT domain may comprise one or more mutations selected from the group consisting of a P51L mutation, a S67K mutation, an E69K mutation, an L139P mutation, a T197A mutation, a D200N mutation, a H204R mutation, A F209N mutation, an E302K mutation, a T306K mutation, a F309N mutation, a W313F mutation, a T330P mutation, an L435G mutation, a P448A mutation, a D449G mutation, an N454K mutation, a D524G mutation, an E562Q mutation, a D583N mutation, an H594Q mutation, an L603W mutation, an E607K mutation, a G615 mutation, an H634Y mutation, a G637R mutation, an H638G mutation, a D653N mutation, or an L671P mutation relative to the reference M-MLV RT as set forth in SEQ ID NO:855. In some embodiments, a DNA polymerase domain, e.g., a RT domain may comprise a mutant RT domain may comprise one or more mutations selected from D200N/T330P/L603W, T306K, W313F, L139P, E607K relative to the reference M-MLV RT as set forth in SEQ ID NO:855.

### Conserved Catalytic Residues

In some embodiments, the prime editor comprises a DNA polymerase domain, e.g., a reverse transcriptase domain that is modified, e.g., by insertion, deletion, or substitution. In some embodiments, the modified DNA polymerase domain, e.g., a reverse transcriptase domain includes one or more amino acid mutations that are located outside the catalytic domains of the polymerase, e.g., reverse transcriptase. In some embodiments, the modified polymerase, e.g., reverse transcriptase, comprises amino acid mutations (e g, amino acid substitutions, deletions, insertions, or chemical modifications located at any position other than the invariant residues, e.g., conserved catalytic residues. In some embodiments, the conserved catalytic residue is an aspartate amino acid, e.g., catalytic aspartate amino acid. In some embodiments, the catalytic aspartate amino acid is involved in incorporation of the correct nucleotide. In some embodiments, mutating an invariant residue results in at least 10%, at least 20%, at least 30%, at least 40%, at least 50%, at least 60%, at least 70%, at least 80%, at least 90%, at least 95%, at least 96%, at least 97%, at least 98%, or at least 99% loss of DNA polymerase, e.g., reverse transcriptase function. In some embodiments, mutating an invariant residue results in 100% loss of DNA polymerase, e.g., reverse transcriptase function. In some embodiments, the amino acid sequence of a DNA polymerase, e.g., reverse transcriptase may be aligned with the amino acid sequence of the reference moloney murine leukemia virus reverse transcriptase (SEQ ID NO: 855) to identify a conserved catalytic residue present in the DNA polymerase, e.g., reverse transcriptase (Table 21). Exemplary conserved catalytic residues are shown in underline in the reference moloney murine leukemia virus reverse transcriptase.

(SEQ ID NO: 855)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLIENSSP

In some embodiments, the amino acid sequence of a reverse transcriptase, e.g., a reference moloney murine leukemia virus RT, e.g., SEQ ID NO: 855, may comprise one or more of D150, D224, and/or D225 conserved catalytic residues. In some embodiments, the amino acid sequence of a reverse transcriptase, may comprise one or more of conserved catalytic residues, e.g., conserved aspartate catalytic residues at positions relative to amino acid residues D150, D225, and/or D225 in a corresponding reference moloney murine leukemia virus reverse transcriptase (SEQ ID NO: 855). In some embodiments, the amino acid sequence of a reverse transcriptase, e.g., a retronb_7, e.g., SEQ ID NO: 18 may comprise one or more of D113, D191, and/or D192 conserved catalytic residues. In some embodiments, the amino acid sequence of a reverse transcriptase, e.g., a Retron_C10, e.g., SEQ ID NO: 16 may comprise one or more of D72, D159, and/or D160 conserved catalytic residues. In some embodiments, the amino acid sequence of a reverse transcriptase, e.g., a spuma_C4, e.g., SEQ ID NO: 261 may comprise one or more of D152, D214, and/or D215 conserved catalytic residues. In some embodiments, the amino acid sequence of a reverse transcriptase, e.g., a spuma_E3, e.g., SEQ ID NO: 270 may comprise one or more of D152, D156, D214, and/or D215 conserved catalytic residues. Table 21 shows exemplary conserved catalytic amino acid residues for some reverse transcriptase domains. In some embodiments, a prime editor comprises a reverse transcriptase variant derived from a reverse transcriptase shown in Table 21 and comprise one or more amino acid substitutions compared to the reverse transcriptase in Table 21, wherein the one or more amino acid substitutions does not include a substitution at a conserved catalytic residue shown in Table 21.

Table 21 shows the exemplary conserved amino acid residues for some reverse transcriptase domains

| SEQ ID NO. | Name | Catalytic Residues |
|---|---|---|
| 855 | reference moloney | D150, D224, D225 |
| | murine leukemia virus | |
| 16 | Retron_C10 | D72, D159, D160 |
| 18 | Retron_B7 | D113, D191, D192 |
| 261 | Spuma_C4 | D152, D214, D215 |
| 270 | Spuma_E3 | D152, D156, D214, D215, |

In some embodiments, the RT or RT domain can be an RT variant. In some embodiments, a prime editor comprises a DNA polymerase domain that is an RT variant. In some embodiments, the RT domain is a RT variant. The RT variant may be a functional fragment of a reference RT (e.g., a RT set forth in SEQ ID NO: 855, or an RT domain, for example, provided in Tables 1, 2, 3, 7, and 14) that has at least 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes (e.g., amino acid substitution and/or amino acid deletion) compared to a reference RT, (e.g., a RT set forth in SEQ ID NO: 855, or a RT set forth in SEQ ID NO: 856, or a RT domain, for example, provided in Tables 1, 2, 3, 7, and 14). In some embodiments, the RT variant comprises a fragment of a reference RT, e.g., a RT set forth in SEQ ID NO: 855, a RT set forth in 856, or an RT domain, for example, provided in Table 1, 2, 3, 7, and 14, such that the fragment is at least about 50% identical, about 60%, identical, about 70% identical, about 80% identical, about 90% identical, about 95% identical, about 96% identical, about 97% identical, about 98% identical, about 99% identical, about 99.5% identical, or about 99.9% identical to the corresponding fragment of the reference RT e.g., a RT set forth in SEQ ID NO: 855, a RT set forth in SEQ ID NO: 856, or an RT domain, for example, provided in Table 1, 2, 3, 4, 7, and 14. In some embodiments, the fragment is at least 30%, 35%, 40%, 45%, 50%, 55%, 60%, 65%, 70%, 75%, 80%, 85%, 90%, 95% identical, 96%, 97%, 98%, 99%, or 99.5% of the amino acid length of a reference sequence, e.g., M-MLV reverse transcriptase set forth in set forth in SEQ ID NO: 855, a RT set forth in SEQ ID NO: 856, an RT provided in Tables 1, 2, 3, 4, 7, and 14.

In some embodiments, the RT functional fragment is at least 100 amino acids in length. In some embodiments, the RT functional fragment is at least 100, 150, 200, 250, 300, 350, 400, 450, 500, 550, or up to 600 or more amino acids in length.

In still other embodiments, a RT variant (e.g., a RT functional fragment) is a RT truncated variant that is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT, e.g., a MMLV RT set forth in SEQ ID NO: 855), a RT set forth in SEQ ID NO: 856, or a RT domain, for example, provided in Tables 1, 3, 7, and 14. In some embodiments, the reference RT is a M-MLV RT set forth in SEQ ID NO: 855. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT, e.g., a M-MLV RT set forth in SEQ ID NO: 855, a RT set forth in SEQ ID NO: 856, or a RT domain, for example, provided in Tables 1, 2, 3, 4, 7, and 14. In some embodiments, the reference RT is a M-MLV RT sequence set forth in SEQ ID NO: 855. In still other embodiments, the RT truncated variant has a truncation at the N-terminal and the C-terminal end compared to a reference RT, e.g., a M-MLV-RT of SEQ ID NO: 855, a RT set forth in SEQ ID NO: 856, or a RT domain, for example, provided in Tables 1, 2, 3, 4, 7, and 14. In some embodiments, the N-terminal truncation and the C-terminal truncation are of the same length. In some embodiments, the N-terminal truncation and the C-terminal truncation are of different lengths.

In some embodiments, the prime editors may include a functional variant of a reference M-MLV reverse transcriptase (e.g., as set forth in SEQ ID NO: 855). In some embodiments, the prime editors comprises a RT domain provided in Tables 1, 2, 3, 4, 7, and 14. In some embodiments, the RT or RT domain is a functional variant of a reference M-MLV RT (e.g., as set forth in SEQ ID NO: 855), a RT set forth in SEQ ID NO: 856, or a RT domain provided in Tables 1, 2, 3, 4, 7, and 14. In some embodiments, the functional variant of M-MLV RT is truncated after amino acid position 502 compared to a M-MLV RT as set forth in SEQ ID NO: 855, a RT set forth in SEQ ID NO: 856, or a RT domain provided in Tables 1, 2 3, 4, 7, and 14. In some embodiments, the functional variant of M-MLV RT further comprises a D200X, T306X, W313X, and/or T330X amino acid substitution compared to a reference M-MLV RT as set forth in SEQ ID NO: 855, or a RT domain provided in Tables 1, 2, 3, 7, and 14, wherein X is any amino acid other than the original amino acid. In some embodiments, the functional variant of M-MLV RT further comprises a D200N, T306K, W313F, and/or T330P amino acid substitution compared to a reference M-MLV RT as set forth in SEQ ID NO: 855, or a RT domain provided in Tables 1, 2, 3, 7, and 14 wherein X is any amino acid other than the original amino acid. A DNA sequence encoding a prime editor comprising this truncated RT is 522 bp smaller than PE2, and therefore makes its potentially useful for applications where delivery of the DNA sequence is challenging due to its size (i.e., adeno-associated virus and lentivirus delivery). In some embodiments, the M-MLV RT variant consists of the following amino acid sequence:

(SEQ ID NO: 884)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFNEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDNSRLIN.

In some embodiments, the reverse transcriptase domain comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any one of the sequences set forth in SEQ ID NO: 5, 6, 13, 15, 16, 17, 18, 21, 22, 130, 131, 204, 230, 232-244, 249-257, 261, 270, 271, 327, 329, 332, 333, 337, 340, 341, 342, 344, 489, 990-1006, 209, 210, 231, 229 provided in Tables 1, 2, or 7. In some embodiments, the reverse transcriptase domain comprises an amino acid sequence identical to any one of the sequences set forth in SEQ ID NO: 5, 6, 13, 15, 16, 17, 18, 21, 22, 130, 131, 204, 230, 232-244, 249-257, 261, 270, 271, 327, 329, 332, 333, 337, 340, 341, 342, 344, 489, 990-1006, 209, 210, 231, 229 (Tables 1, 2). Exemplary reverse transcriptase domains are shown in Tables 1, 2, 3, 7, and 14.

In some embodiments, the RT domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NOs: 1-95, 198-271, 319-493, 533-846, 855-857, 884, or 990-1006. In some embodiments, the RT domain comprises an amino acid sequence that is selected from the group consisting of: SEQ ID NOs: 1-95, 198-271, 319-493, 533-846, 855-857, 884, or 990-1006. In some embodiments, the RT domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the amino acid sequences set forth in SEQ ID NO: 1-95, 198-271, 319-493, 533-846, 855-857, 884, or 990-1006.

RT Families

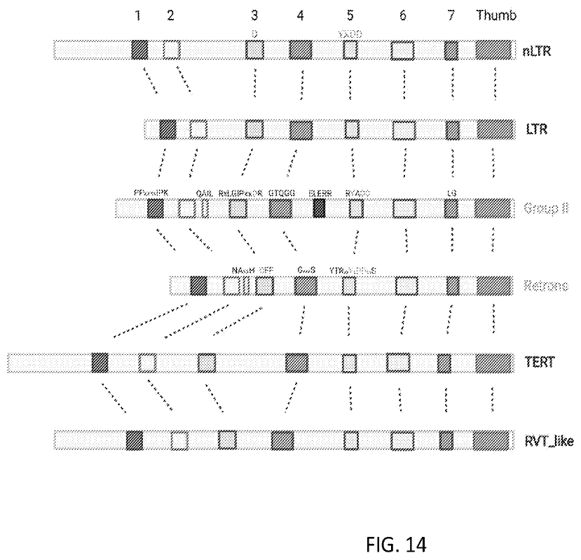

RT domains used in prime editors can comprise multiple functional domains. In some embodiments, an RT domain may comprise a domain 1, a domain 2, a domain 3, a domain 4, a domain 5, a domain 6, a domain 7, and/or a Thumb domain. In some embodiments, a first polypeptide may comprise a domain 1, a domain 2, a domain 3, a domain 4, a domain 5, a domain 6, a domain 7, or a Thumb domain. In some embodiments, a domain 1, a domain 2, a domain 3, a domain 4, a domain 5, a domain 6, a domain 7, or a Thumb domain may also be part of a DNA polymerase domain, e.g., an RNA-mediated DNA polymerase domain. In some embodiments, a plurality of RT domains may share the domain structure of domain 1, domain 2, domain 3, domain 4, domain 5, domain 6, domain 7, and the Thumb domain. The plurality of RT domains may be grouped into a plurality of RT families based on a specific sequence or structure feature in any of the domains thereof. In some embodiments, a plurality of RT domains may be grouped into six families as described in FIG. 14. In some embodiments, a method to classify the RT domains based on the domain structure thereof is described in Example 6.

In some embodiments, a DNA polymerase domain in a prime editor may be modified compared to a wild type form. For example, a prime editor may comprise a truncated RT domain. In some embodiments, one or more domains of a naturally occurring RT is truncated or reduced for use in a prime editor. In some embodiments, the RT is a retro viral RT (e.g., MMLV-RT) wherein a RNaseH domain of the wild type retroviral RT is truncated or deleted. In some embodiments, amino acid sequences connecting one or more of domain 1 and domain 2, domain 2 and domain 3, domain 3 and domain 4, domain 4 and domain 5, domain 5 and domain 6, domain 6 and domain 7, or domain 7 and thumb domain of a naturally occurring RT may be truncated or deleted for use in a prime editor.

In some embodiments, an RT domain of a prime editor may be selected from the group consisting of an nLTR RT domain, an LTR RT domain, a Group II intron RT domain, a Retron RT domain, a TERT RT domain, and an RVT_like RT domain. In some embodiments, an RT domain may be selected from the group consisting of a nLTR RT domain, an LTR RT domain, a Group II intron RT domain, a Retron RT domain, a TERT RT domain, and an RVT_like RT domain. In some embodiments, an RT domain of a prime editor may comprise an nLTR RT domain. In some embodiments, an RT domain of a prime editor may comprise an LTR RT domain. In some embodiments, an RT domain of a prime editor may comprise a Group II intron RT domain. In some embodiments, an RT domain of a prime editor may comprise a Retron RT domain. In some embodiments, an RT domain of a prime editor may comprise a TERT RT domain. In some embodiments, an RT domain of a prime editor may comprise an RVT_like RT domain. In some embodiments, a DNA polymerase domain or an RNA-mediated DNA polymerase domain of a prime editor may comprise the RT domain thereof or any combinations described herein.

In some embodiments, a prime editor comprises an RT domain comprising an aspartic acid in domain 3. In some embodiments, a prime editor comprises an RT domain comprising the amino acid sequence YxDD in domain 5, wherein x is any amino acid. In some embodiments, a prime editor comprises an RT domain comprising an aspartic acid in domain 3 and the amino acid sequence YxDD in domain 5, wherein x is any amino acid. In some embodiments, the RT domain is a nLTR RT domain. An nLTR RT domain of a prime editor may comprise any combinations of the amino acid or sequence described herein. The amino acid or sequence described herein may also apply to an nLTR RT domain of an RT or a polypeptide. The amino acid or sequence described herein may not be restricted to the nLTR RT domain of a prime editor.

In some embodiments, a prime editor comprises an RT domain comprising the amino acid sequence PPxxxxIPK(SEQ ID NO: 905) in domain 1, wherein x is any amino acid. In some embodiments, a prime editor comprises an RT domain comprising the amino acid sequence QAIL (SEQ ID NO: 906) at position between domain 2 and domain 3. In some embodiments, a prime editor comprises an RT domain comprising the amino acid sequence RxLGIPxxDR (SEQ ID NO: 907) in domain 3, wherein x is any amino acid. In some embodiments, the prime editor comprises an RT domain comprising the amino acid sequence GTQGG (SEQ ID NO: 908) in domain 4. In some embodiments, the prime editor comprises an RT domain comprising the amino acid sequence ELERR (SEQ ID NO: 909) between domain 4 and domain 5 In some embodiments, the prime editor comprises an RT domain comprising the amino acid sequence LG in domain 7 In some embodiments, a prime editor comprises an RT domain comprising the amino acid sequence PPxxxxIPK (SEQ ID NO: 905) in domain 1, the amino acid sequence QAIL (SEQ ID NO: 906) at position between domain 2 and domain 3. In some embodiments, the amino acid sequence RxLGIPxxDR (SEQ ID NO: 907) in domain 3, the amino acid sequence GTQGG (SEQ ID NO: 908) in domain 4, the amino acid sequence ELERR(SEQ ID NO: 909) between domain 4 and domain 5, and/or the amino acid sequence LG in domain 7, or any combination thereof, where x is any amino acid. In some embodiments, the RT domain is a Group II intron RT domain. A Group II intron RT domain of a prime editor may comprise any combinations of the amino acid or sequence described herein. The amino acid or sequence described herein may also apply to a Group II intron RT domain of an RT or a polypeptide. The amino acid or sequence described herein may not be restricted to the Group II intron RT domain of a prime editor.

In some embodiments, a prime editor may comprise a RT domain comprising the amino acid sequence NAxxH between domain 2 and domain 3, wherein x is any amino acid. In some embodiments, the prime editor comprises the amino acid sequence DFF in domain 3; GxxS in domain 4, wherein x is any amino acid; and/or YTRxxYxxDDxxS in domain 5, wherein x is any amino acid. In some embodiments, the prime editor comprises a RT domain comprising the amino acid sequence NAxxH (SEQ ID NO: 910) between domain 2 and domain 3, wherein x is any amino acid. In some embodiments, the prime editor comprises a RT domain comprising the amino acid sequence DFF in domain 3; or GxxS in domain 4 In some embodiments, the prime editor comprises a RT domain comprising the amino acid sequence YTRxxYxxDDxxS (SEQ ID NO: 910) in domain 5, wherein x is any amino acid. In other embodiments, the prime editor comprises a RT domain comprising the amino acid sequence NAxxH between domain 2 and domain 3; DFF at position in domain 3; GxxS in domain 4, and/or YTRxxYxxDDxxS (SEQ ID NO: 910) in domain 5, wherein x is any amino acid. In some embodiments, the RT domain is a Retron RT domain of a prime editor may comprise any combinations of the amino acid or sequence described herein. The amino acid or sequence described herein to a Retron RT domain of an RT or a polypeptide. The amino acid or sequence described herein may not be restricted to the Retron RT domain of a prime editor.

In some embodiments, a prime editor comprises an eukaryotic RT, for example, a yeast, drosophila, rodent, or primate RT. In some embodiments, the RT or RT domain is an eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the RT or RT domain is a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT. In some embodiments, RT or RT domain comprises a retron RT.

### Ancestral Reverse Transcriptase

Components of prime editors described herein may comprise engineered protein sequence that share evolutionary ancestors with currently known proteins. For example, a prime editor may comprise a DNA polymerase that is reverse transcriptase (RT) polypeptide that comprises an ancestral sequence of a family of RTs. Sequences from National Center for Biotechnology Information (NCBI), UniProt, EMBL, International Nucleotide Sequence Database Collaboration (INSDC), European Nucleotide Archive, or other databases may be used to construct ancestral sequences. The collected sequences may be aligned by a multiple sequence alignment (MSA) algorithm. An MSA alignment algorithm may ClustalW, Kalign, MAFFT, MUSCLE, T-Coffee, derivatives thereof, or any combinations thereof. Methods to handle gaps in sequence alignments may comprise Probabilistic Alignment Kit (PRANK) or any derivatives thereof. Methods to handle gaps in sequence alignments, in some embodiments, may also comprise RaxML. In some embodiments, an evolutionary model may be used to construct an ancestral phylogeny tree. An evolutionary model may comprise Dayhoff models, for example, PAM120, PAM160, PAM250, or any derivatives thereof. An evolutionary model may also comprise the ITT model, the WAG model, the LG model, the R10 model, the INV model, or the Blosum models. A Blosum model may comprise Blosum45, Blosum62, Blosum80, or any derivatives thereof. In some embodiments, an evolutionary model may comprise computational constraints on the structure or function of the sequences. The constraints may be imposed by a computational model. The fitness of an evolutionary model may also be evaluated using the Aikake Information Criterion or the Bayesian Information Criterion. In some embodiments, a phylogenetic tree may be constructed once the evolutionary model and its fitness are calculated. In some embodiments, a phylogenetic tree may comprise maximum likelihood methods. A maximum likelihood method may comprise PhyML, MOLPHY, BioNJ, PHYLIP, or any derivatives thereof.

In some embodiments, an RT domain (e.g., an engineered RT) comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to any one of sequences selected from the group consisting of: SEQ ID NOs: 81-95. In some embodiments, an RT domain (e.g., an engineered RT) comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to any one of sequences selected from the group consisting of: SEQ ID NOs: 81, 82, 84, 91. In some embodiments, an RT domain (e.g., an engineered RT) comprises an amino acid sequence that has no more than 1, 2, 3, 4, 5, 6, 7, 8, 9, 10, 11, 12, 13, 14, 15, 16, 17, 18, 19, 20, 25, 30, 35, 40, ti, 45, or 50 differences e.g., mutations e g, amino acid deletions, amino acid substitutions, or amino acid substitutions compared to any of the amino acid sequences set forth in SEQ ID NO: 81-95. In some embodiments, an RT domain (e.g., an engineered RT) comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., amino acid deletions, amino acid substitutions, or amino acid substitutions compared to any of the amino acid sequences set forth in SEQ ID NO: 81, 82, 84, 91. In some embodiments, the RT domain comprises an amino acid sequence that is selected from any one of sequences set forth in SEQ ID NOs: 81-95. In some embodiments, the RT domain comprises an amino acid sequence that is selected from any one of sequences set forth in SEQ ID NOs: 81, 82, 84, 91.

In some embodiments, a method of reverse transcribing a target RNA sequence may comprise contacting a target RNA sequence with an RT domain described herein. In some embodiments, the RT domain may reverse transcribe the RNA molecule into a complementary DNA sequence. In some embodiments, a cell may comprise the RT domains described herein.

In some embodiments, the RT domains described herein may comprise any SET domains described herein. In some embodiments, a composition may comprise the RT domains described herein. In some embodiments, a kit may also comprise the RT domains described herein.

The solubility of a prime editor in vitro may be measured by expressing the prime editor in bacteria as a recombinant protein, disrupting the bacteria, centrifugation the bacterial lysate into a supernatant and pellet. The amount of protein in these fractions may be visualized and quantified using western blotting. The amount of protein in the supernatant represents the soluble fraction, and the amount of protein in the pellet represents the insoluble fraction. The solubility of a prime editor in vivo may be measured by a split GFP assay as follows: A 15-amino-acid GFP fragment, GFP 11, is fused to the prime editor and expressed in a host cell. The GFP 1-10 detector fragment is expressed separately in the host cell. These fragments associate spontaneously to form fluorescent GFP if the prime editor comprising the GFP 11 fragment is soluble. The amount of GFP fluorescence of the host cell is proportional to the solubility of the prime editor in vivo. The expression level of a prime editor in vitro may be measured by expressing the prime editor in bacteria as a recombinant protein and lysing the bacteria. The amount of protein in the bacterial lysate may be visualized and quantified using western blotting. The expression level of a prime editor in vivo may be measured by expressing the prime editor in host cells and lysing the cells. The amount of protein in the cell lysate may be visualized and quantified using western blotting. The prime editing efficiency may be measured by the methods described in Examples 2-5 and. The DNA polymerase activity may be measured by conversion of radiolabeled deoxyribonucleoside triphosphate into an acid-insoluble product as follows: A DNA template primed with a primer is incubated with the radiolabeled deoxyribonucleoside triphosphates and a prime editor. The reaction is stopped by chilling and addition of perchloric acid. The acid-insoluble radioactivity is determined and is proportional to the DNA polymerase activity. The DNA-binding activity may be measured by an electrophoretic mobility shift assay. The DNA endonuclease activity may be measured by incubating a purified prime editor or a lysate of a cell expressing a prime editor with a cleavage target DNA; and measuring the cleavage product by agarose electrophoresis. Other methods or derivations of the methods described herein and known by a skilled in the art may also be used. The methods described herein may also be used to measure the solubility, expression level, DNA-binding activity, DNA endonuclease activity of any engineered RT described herein. The DNA polymerase may be used to measure the RT activity of any engineered RT described herein. For example, an oligo-dT primer may be used to prime an RNA template in a RT reaction to measure the RT activity.

Prime Editors with Solubility Enhancement (SET) Domains

A prime editor described herein may comprise additional functional domains, for example, one or more domains that modify the folding, solubility, or charge of the prime editor. In some embodiments, the prime editor may comprise a SET domain.

A SET domain may be associated, linked, or fused to any component of a prime editor (e.g., to a DNA polymerase domain and/or a DNA binding domain). In some embodiments, a SET domain is linked to a DNA-binding domain of a prime editor. In some embodiments, a SET domain is linked to a DNA polymerase domain of a prime editor. In some embodiments, where the prime editor is a fusion protein, the SET domain may be positioned at the N-terminus of the prime editor, the C-terminus of the prime editor, or in between a DNA binding domain and a polymerase domain.

In some embodiments, a SET domain may increase the solubility of a prime editor in vitro, relative to a prime editor without the SET domain. In some embodiments, the SET domain may increase the solubility of a prime editor in vivo, relative to a prime editor without the SET domain. The increase in solubility of a prime editor comprising the SET domain relative to a prime editor without the SET domain, in some embodiments, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in solubility of a prime editor comprising the SET domain relative to a prime editor without the SET domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in solubility of a prime editor comprising the SET domain relative to a prime editor without the SET domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in solubility of a prime editor comprising the SET domain relative to a prime editor without the SET domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, the SET domain may increase the expression level of a prime editor in vitro, relative to a prime editor without the SET domain. In some embodiments, the SET domain may increase the expression level of a prime editor in vivo, relative to a prime editor without the SET domain. The increase in expression level of a prime editor comprising the SET domain relative to a prime editor without the SET domain, in some embodiments, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in expression level of a prime editor comprising the SET domain relative to a prime editor without the SET domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in expression level of a prime editor comprising the SET domain relative to a prime editor without the SET domain, in some embodiments, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in expression level of a prime editor comprising the SET domain relative to a prime editor without the SET domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a prime editor comprising the SET domain may increase prime editing efficiency, DNA polymerase activity, DNA-binding activity, or DNA endonuclease activity relative to a prime editor without the SET domain. The increase in the prime editing efficiency, DNA polymerase activity, DNA-binding activity, or DNA endonuclease activity of a prime editor comprising the SET domain relative to a prime editor without the SET domain, in some embodiments, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in the prime editing efficiency, DNA polymerase activity, DNA-binding activity, or DNA endonuclease activity of a prime editor comprising the SET domain relative to a prime editor without the SET domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in the prime editing efficiency, DNA polymerase activity, DNA-binding activity, or DNA endonuclease activity of a prime editor comprising the SET domain relative to a prime editor without the SET domain, in some embodiments, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in the prime editing efficiency, DNA polymerase activity, DNA-binding activity, or DNA endonuclease activity of a prime editor comprising the SET domain relative to a prime editor without the SET domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a SET domain may adopt a secondary, tertiary, or quaternary structure when not fused to other components of the prime editor. In some embodiments, the SET domain may adopt a secondary structure without the prime editor. In some embodiments, the SET domain of a prime editor may adopt a tertiary structure without the prime editor. In some embodiments, the SET domain of a prime editor may adopt a quaternary structure without the prime editor. The SET domain of a prime editor adopting a secondary, tertiary, or quaternary structure without the prime editor may comprise any size described herein.

In some embodiments, the SET domain the SET domain of a prime editor may be less than about 100 kDa (kilo Dalton) or may adopt a secondary, tertiary, or quaternary structure without the prime editor. In some embodiments, the SET domain of a prime editor may be less than about 100 kDa. In some embodiments, the SET domain of a prime editor may be less than about 100 kDa and adopt a secondary, tertiary, or quaternary structure without the prime editor. In some embodiments, the SET domain of a prime editor may be less than about 50 kDa or may adopt a secondary, tertiary, or quaternary structure without the prime editor. In some embodiments, the SET domain of a prime editor may be less than about 50 kDa. In some embodiments, the SET domain of a prime editor may be less than about 50 kDa and adopt a secondary, tertiary, or quaternary structure without the prime editor.

In some embodiments, the SET domain of a prime editor may be less than about 20 kDa or may adopt a secondary, tertiary, or quaternary structure without the prime editor. In some embodiments, the SET domain of a prime editor may be less than about 20 kDa. In some embodiments, the SET domain of a prime editor may be less than about 20 kDa and adopt a secondary, tertiary, or quaternary structure without the prime editor. In some embodiments, the SET domain of a prime editor may be less than about 10 kDa or may adopt a secondary, tertiary, or quaternary structure without the prime editor. In some embodiments, the SET domain of a prime editor may be less than about 10 kDa. In some embodiments, the SET domain of a prime editor may be less than about 10 kDa and adopt a secondary, tertiary, or quaternary structure without the prime editor. In some embodiments, the SET domain of a prime editor may be less than about 9 kDa or may adopt a secondary, tertiary, or quaternary structure without the prime editor. In some embodiments, the SET domain of a prime editor may be less than about 9 kDa. In some embodiments, the SET domain of a prime editor may be less than about 9 kDa and adopt a secondary, tertiary, or quaternary structure without the prime editor. In some embodiments, the SET domain of a prime editor may be less than about 8 kDa or may adopt a secondary, tertiary, or quaternary structure without the prime editor. In some embodiments, the SET domain of a prime editor may be less than about 8 kDa. In some embodiments, the SET domain of a prime editor may be less than about 8 kDa and adopt a secondary, tertiary, or quaternary structure without the prime editor.

In some embodiments, the SET domain of a prime editor may be less than about 7 kDa or may adopt a secondary, tertiary, or quaternary structure without the prime editor. In some embodiments, the SET domain of a prime editor may be less than about 7 kDa. In some embodiments, the SET domain of a prime editor may be less than about 7 kDa and adopt a secondary, tertiary, or quaternary structure without the prime editor.

In some embodiments, the SET domain of a prime editor may be less than about 6 kDa or may adopt a secondary, tertiary, or quaternary structure without the prime editor. In some embodiments, the SET domain of a prime editor may be less than about 6 kDa. In some embodiments, the SET domain of a prime editor may be less than about 6 kDa and adopt a secondary, tertiary, or quaternary structure without the prime editor. In some embodiments, the SET domain of a prime editor may be less than about 5 kDa or may adopt a secondary, tertiary, or quaternary structure without the prime editor. In some embodiments, the SET domain of a prime editor may be less than about 5 kDa. In some embodiments, the SET domain of a prime editor may be less than about 5 kDa and adopt a secondary, tertiary, or quaternary structure without the prime editor.

In some embodiments, the SET domain of a prime editor may be less than about 95 kDa, less than about 90 kDa, less than about 85 kDa, less than about 80 kDa, less than about 75 kDa, less than about 70 kDa, less than about 65 kDa, 60 kDa, or less than about 55 kDa; or may adopt a secondary, tertiary, or quaternary structure without the prime editor. In some embodiments, the SET domain of a prime editor may be less than about 95 kDa, less than about 90 kDa, less than about 85 kDa, less than about 80 kDa, less than about 75 kDa, less than about 70 kDa, less than about 65 kDa, 60 kDa, or less than about 55 kDa. In some embodiments, the SET domain of a prime editor may be less than about 95 kDa, less than about 90 kDa, less than about 85 kDa, less than about 80 kDa, less than about 75 kDa, less than about 70 kDa, less than about 65 kDa, 60 kDa, or less than about 55 kDa; and adopt a secondary, tertiary, or quaternary structure without the prime editor. In some embodiments, the SET domain of a prime editor may be less than about 45 kDa, less than about 40 kDa, less than about 35 kDa, less than about 30 kDa; or may adopt a secondary, tertiary, or quaternary structure without the prime editor. In some embodiments, the SET domain of a prime editor may be less than about 45 kDa, less than about 40 kDa, less than about 35 kDa, less than about 30 kDa. In some embodiments, the SET domain of a prime editor may be less than about 45 kDa, less than about 40 kDa, less than about 35 kDa, less than about 30 kDa; and adopt a secondary, tertiary, or quaternary structure without the prime editor. In some embodiments, the SET domain of a prime editor may be less than about 11 kDa, less than about 12 kDa, less than about 13 kDa, less than about 14 kDa, less than about 15 kDa, less than about 16 kDa, less than about 17 kDa, less than about 18 kDa, or less than about 19 kDa; or may adopt a secondary, tertiary, or quaternary structure without the prime editor. In some embodiments, the SET domain of a prime editor may be less than about 11 kDa, less than about 12 kDa, less than about 13 kDa, less than about 14 kDa, less than about 15 kDa, less than about 16 kDa, less than about 17 kDa, less than about 18 kDa, or less than about 19 kDa. In some embodiments, the SET domain of a prime editor may be less than about 11 kDa, less than about 12 kDa, less than about 13 kDa, less than about 14 kDa, less than about 15 kDa, less than about 16 kDa, less than about 17 kDa, less than about 18 kDa, or less than about 19 kDa; and adopt a secondary, tertiary, or quaternary structure without the prime editor. In some embodiments, the SET domain of a prime editor may be less than about 4 kDa, less than about 3 kDa, less than about 2 kDa, or less than about 1 kDa; or may adopt a secondary, tertiary, or quaternary structure without the prime editor. In some embodiments, the SET domain of a prime editor may be less than about 4 kDa, less than about 3 kDa, less than about 2 kDa, or less than about 1 kDa. In some embodiments, the SET domain of a prime editor may be less than about 4 kDa, less than about 3 kDa, less than about 2 kDa, or less than about 1 kDa; and adopt a secondary, tertiary, or quaternary structure without the prime editor.

In some embodiments, the SET domain of a prime editor may comprise a GB1 domain, a protein D domain, a Z domain of Staphylococcal protein A, a Fhb domain, an MBP domain, a NusA domain, a Trx domain, a SUMO domain, a GST domain, a GB1 domain, a ZZ domain, a HaloTag domain, a SNUT domain, a Skp domain, a T7PK domain, an EspA domain, a Mocr domain, an Ecotin domain, a CaBP domain, an ArsC domain, an IF2-domain I domain, a RpoA domain, a SlyD domain, a Tsf domain, a RpoS domain, a PotD domain, a Crr domain, a msyB domain, an yjgD domain, a rpoD domain, a GFP domain, or a AK-tag domain. In some embodiments, the SET domain of a prime editor may comprise a protein D domain. In some embodiments, the SET domain of a prime editor may comprise a Z domain of Staphylococcal protein A. In some embodiments, the SET domain of a prime editor may comprise a Fhb domain. In some embodiments, the SET domain of a prime editor may comprise an MBP domain. In some embodiments, the SET domain of a prime editor may comprise a NusA domain. In some embodiments, the SET domain of a prime editor may comprise, a Trx domain. In some embodiments, the SET domain of a prime editor may comprise a SUMO domain. In some embodiments, the SET domain of a prime editor may comprise a GST domain. In some embodiments, the SET domain of a prime editor may comprise a GB1 domain. In some embodiments, the SET domain of a prime editor may comprise a ZZ domain. In some embodiments, the SET domain of a prime editor may comprise a HaloTag domain. In some embodiments, the SET domain of a prime editor may comprise a SNUT domain. In some embodiments, the SET domain of a prime editor may comprise a Skp domain. In some embodiments, the SET domain of a prime editor may comprise a T7PK domain. In some embodiments, the SET domain of a prime editor may comprise an EspA domain. In some embodiments, the SET domain of a prime editor may comprise a Mocr domain. In some embodiments, the SET domain of a prime editor may comprise an Ecotin domain. In some embodiments, the SET domain of a prime editor may comprise a CaBP domain. In some embodiments, the SET domain of a prime editor may comprise an ArsC domain. In some embodiments, the SET domain of a prime editor may comprise an IF2-domain I domain. In some embodiments, the SET domain of a prime editor may comprise a RpoA domain. In some embodiments, the SET domain of a prime editor may comprise a SlyD domain. In some embodiments, the SET domain of a prime editor may comprise a Tsf domain. In some embodiments, the SET domain of a prime editor may comprise a RpoS domain. In some embodiments, the SET domain of a prime editor may comprise a PotD domain. In some embodiments, the SET domain of a prime editor may comprise a Crr domain. In some embodiments, the SET domain of a prime editor may comprise a msyB domain. In some embodiments, the SET domain of a prime editor may comprise an yjgD domain. In some embodiments, the SET domain of a prime editor may comprise a rpoD domain. In some embodiments, the SET domain of a prime editor may comprise a GFP domain. In some embodiments, the SET domain of a prime editor may comprise an AK-tag domain.

In some embodiments, a SET domain of a prime editor comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NOs: 96-124 or 137. In some embodiments, a SET domain (e.g., an engineered RT) comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the ammo acid sequences set forth in SEQ ID NO: 96-124 or 137. In some embodiments, a SET domain of a prime editor comprises an amino acid sequence that is selected from the group consisting of: SEQ ID NOs: 96-124 or 137. In some embodiments, the SET domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NO: 102 and SEQ ID NO: 137. In some embodiments, a SET domain (e.g., an engineered RT) comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the amino acid sequences set forth in SEQ ID NO: 102 and SEQ ID NO: 137. In some embodiments, a SET domain of a prime editor comprises an amino acid sequence selected from the group consisting of: SEQ ID NO: 102 and SEQ ID NO: 137. In some embodiments, the SET domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence set forth in SEQ ID NO: 102. In some embodiments, a SET domain (e.g., an engineered RT) comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to an amino acid sequences set forth in SEQ ID NO: 102. In some embodiments, a SET domain of a prime editor comprises an amino acid sequence set forth at SEQ ID NO: 102. In some embodiments, the SET domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence set forth in SEQ ID NO: 137. In some embodiments, a SET domain (e.g., an engineered RT) comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to an amino acid sequences set forth in SEQ ID NO: 137. In some embodiments, a SET domain of a prime editor comprises an amino acid sequence set forth at SEQ ID NO: 137.

In some embodiments, a prime editor comprising a SET domain of comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NOs: 125-128. In some embodiments, a prime editor comprising a SET domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the amino acid sequences set forth in SEQ ID NO: 125-128. In some embodiments, a prime editor comprising a SET domain of a prime editor comprises an amino acid sequence that is selected from the group consisting of: SEQ ID NOs: 125-128.

In some embodiments, a SET domain may increase the solubility, the expression level, the prime editing efficiency, the DNA polymerase activity, the DNA-binding activity, or the DNA endonuclease activity of a prime editor, relative to a prime editor lacking the SET domain. In some embodiments, a SET domain may increase the solubility, the expression level, the prime editing efficiency, the DNA polymerase activity, the DNA-binding activity, or the DNA endonuclease activity of a prime editor, relative to a prime editor lacking the SET domain. In some embodiments, a SET domain may increase the solubility of a prime editor, relative to a prime editor lacking the SET domain. In some embodiments, a SET domain may increase the expression level of a prime editor, relative to a prime editor lacking the SET domain. In some embodiments, a SET domain may increase the prime editing efficiency of a prime editor, relative to a prime editor lacking the SET domain. In some embodiments, a SET domain may increase the DNA polymerase activity of a prime editor, relative to a prime editor lacking the SET domain. In some embodiments, a SET domain may increase the DNA-binding activity of a prime editor, relative to a prime editor lacking the SET domain. In some embodiments, a SET domain may increase the DNA endonuclease activity of a prime editor, relative to a prime editor lacking the SET domain.

In some embodiments, a SET domain may increase the solubility of a prime editor in vitro, relative to a prime editor lacking the SET domain. In some embodiments, a SET domain may increase the solubility of a prime editor in vivo, relative to a prime editor lacking the SET domain. The increase in solubility of a prime editor comprising a SET domain relative to a prime editor lacking the SET domain, in some embodiments, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in solubility of a prime editor comprising a SET domain relative to a prime editor lacking the SET domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in solubility of a prime editor comprising a SET domain relative to a prime editor lacking the SET domain, in some embodiments, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in solubility of a prime editor comprising a SET domain relative to a prime editor lacking the SET domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a SET domain may increase the expression level of a prime editor in vitro, relative to a prime editor lacking the SET domain. In some embodiments, a SET domain may increase the expression level of a prime editor in vivo, relative to a prime editor lacking the SET domain. The increase in expression level of a prime editor comprising a SET domain relative to a prime editor lacking the SET domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in expression level of a prime editor comprising a SET domain relative to a prime editor lacking the SET domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in expression level of a prime editor comprising a SET domain relative to a prime editor lacking the SET domain, in some embodiments, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in expression level of a prime editor comprising a SET domain relative to a prime editor lacking the SET domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

The increase in the prime editing efficiency, DNA polymerase activity, DNA-binding activity, or DNA endonuclease activity of a prime editor comprising a SET domain relative to a prime editor lacking the SET domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in the prime editing efficiency, DNA polymerase activity, DNA-binding activity, or DNA endonuclease activity of a prime editor comprising a SET domain relative to a prime editor lacking the SET domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in the prime editing efficiency, DNA polymerase activity, DNA-binding activity, or DNA endonuclease activity of a prime editor comprising a SET domain relative to a prime editor lacking the SET domain, in some embodiments, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in the prime editing efficiency, DNA polymerase activity, DNA-binding activity, or DNA endonuclease activity of a prime editor comprising a SET domain relative to a prime editor lacking the SET domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a SET domain of a prime editor may comprise a GB1 domain, a protein D domain, a Z domain of Staphylococcal protein A, a Fh8 domain, an MBP domain, a NusA domain, a Trx domain, a SUMO domain, a GST domain, a GB1 domain, a ZZ domain, a HaloTag domain, a SNUT domain, a Skp domain, a T7PK domain, an EspA domain, a Mocr domain, an Ecotin domain, a CaBP domain, an ArsC domain, an IF2-domain I domain, a RpoA domain, a SlyD domain, a Tsf domain, a RpoS domain, a PotD domain, a Crr domain, a msyB domain, an yjgD domain, a rpoD domain, a GFP domain, or a AK-tag domain. In some embodiments, a SET domain of a prime editor may comprise a protein D domain. In some embodiments, a SET domain of a prime editor may comprise a Z domain of Staphylococcal protein A. In some embodiments, a SET domain of a prime editor may comprise a Fh8 domain. In some embodiments, a SET domain of a prime editor may comprise an MBP domain. In some embodiments, a SET domain of a prime editor may comprise a NusA domain. In some embodiments, a SET domain of a prime editor may comprise, a Trx domain. In some embodiments, a SET domain of a prime editor may comprise a SUMO domain. In some embodiments, a SET domain of a prime editor may comprise a GST domain. In some embodiments, a SET domain of a prime editor may comprise a GB1 domain. In some embodiments, a SET domain of a prime editor may comprise a ZZ domain. In some embodiments, a SET domain of a prime editor may comprise a HaloTag domain. In some embodiments, a SET domain of a prime editor may comprise a SNUT domain. In some embodiments, a SET domain of a prime editor may comprise a Skp domain. In some embodiments, a SET domain of a prime editor may comprise a T7PK domain. In some embodiments, a SET domain of a prime editor may comprise an EspA domain. In some embodiments, a SET domain of a prime editor may comprise a Mocr domain. In some embodiments, a SET domain of a prime editor may comprise an Ecotin domain. In some embodiments, a SET domain of a prime editor may comprise a CaBP domain. In some embodiments, a SET domain of a prime editor may comprise an ArsC domain. In some embodiments, a SET domain of a prime editor may comprise an IF2-domain I domain. In some embodiments, a SET domain of a prime editor may comprise a RpoA domain. In some embodiments, a SET domain of a prime editor may comprise a SlyD domain. In some embodiments, a SET domain of a prime editor may comprise a Tsf domain. In some embodiments, a SET domain of a prime editor may comprise a RpoS domain. In some embodiments, a SET domain of a prime editor may comprise a PotD domain. In some embodiments, a SET domain of a prime editor may comprise a Crr domain. In some embodiments, a SET domain of a prime editor may comprise a msyB domain. In some embodiments, a SET domain of a prime editor may comprise an yjgD domain. In some embodiments, a SET domain of a prime editor may comprise a rpoD domain. In some embodiments, a SET domain of a prime editor may comprise a GFP domain. In some embodiments, a SET domain of a prime editor may comprise an AK-tag domain.

In some embodiments, the SET domain of a prime editor comprises a GB1 domain. In some embodiments, the SET domain of a prime editor comprises a GB1 domain. In some embodiments, a GB1 domain may increase the solubility, the expression level, the prime editing efficiency, the DNA polymerase activity, the DNA-binding activity, or the DNA endonuclease activity of a prime editor, relative to a prime editor lacking the GB1 domain. In some embodiments, a GB1 domain may increase the solubility of a prime editor, relative to a prime editor lacking the SET domain. In some embodiments, a GB1 domain may increase the expression level of a prime editor, relative to a prime editor lacking the SET domain. In some embodiments, a GB1 domain may increase the prime editing efficiency of a prime editor, relative to a prime editor lacking the SET domain. In some embodiments, a GB1 domain may increase the DNA polymerase activity of a prime editor, relative to a prime editor lacking the SET domain. In some embodiments, a GB1 domain may increase the DNA-binding activity of a prime editor, relative to a prime editor lacking the SET domain. In some embodiments, a GB1 domain may increase the DNA endonuclease activity of a prime editor, relative to a prime editor lacking the SET domain.

In some embodiments, a GB1 domain may increase the solubility of a prime editor in vitro, relative to a prime editor lacking the GB1 domain. In some embodiments, a GB1 domain may increase the solubility of a prime editor in vivo, relative to a prime editor lacking the GB1 domain. The increase in solubility of a prime editor comprising a GB1 domain relative to a prime editor lacking the GB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in solubility of a prime editor comprising a GB1 domain relative to a prime editor lacking the GB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in solubility of a prime editor comprising a GB1 domain relative to a prime editor lacking the GB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in solubility of a prime editor comprising a GB1 domain relative to a prime editor lacking the GB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a GB1 domain may increase the expression level of a prime editor in vitro, relative to a prime editor lacking the GB1 domain. In some embodiments, a GB1 domain may increase the expression level of a prime editor in vivo, relative to a prime editor lacking the GB1 domain. The increase in expression level of a prime editor comprising a GB1 domain relative to a prime editor lacking the GB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in expression level of a prime editor comprising a GB1 domain relative to a prime editor lacking the GB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in expression level of a prime editor comprising a GB1 domain relative to a prime editor lacking the GB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in expression level of a prime editor comprising a GB1 domain relative to a prime editor lacking the GB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a GB1 domain may have an increased prime editing efficiency, DNA polymerase activity, DNA-binding activity, or DNA endonuclease activity of a prime editor comprising a GB1 domain relative to a prime editor lacking the GB1 domain. The increase in the prime editing efficiency, DNA polymerase activity, DNA-binding activity, or DNA endonuclease activity of a prime editor comprising a GB1 domain relative to a prime editor lacking the GB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in the prime editing efficiency, DNA polymerase activity, DNA-binding activity, or DNA endonuclease activity of a prime editor comprising a GB1 domain relative to a prime editor lacking the GB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in the prime editing efficiency, DNA polymerase activity, DNA-binding activity, or DNA endonuclease activity of a prime editor comprising a GB1 domain relative to a prime editor lacking the GB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in the prime editing efficiency, DNA polymerase activity, DNA-binding activity, or DNA endonuclease activity of a prime editor comprising a GB1 domain relative to a prime editor lacking the GB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a GB1 domain of a prime editor comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NOs: 96-124 or 137. In some embodiments, a GB1 domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the amino acid sequences set forth in SEQ ID NO: 96-124 or 137. In some embodiments, a GB1 domain of a prime editor comprises an amino acid sequence that is selected from the group consisting of: SEQ ID NOs: 96-124 or 137. In some embodiments, the GB1 domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NO: 102 and SEQ ID NO: 137. In some embodiments, a GB1 domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the ammo acid sequences set forth in SEQ ID NO: 102 and SEQ ID NO: 137. In some embodiments, a GB1 domain of a prime editor comprises an amino acid sequence selected from the group consisting of: SEQ ID NO: 102 and SEQ ID NO: 137. In some embodiments, the GB1 domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence set forth in SEQ ID NO: 102. In some embodiments, a GB1 domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to an amino acid sequences set forth in SEQ ID NO: 102. In some embodiments, a GB1 domain of a prime editor comprises an amino acid sequence set forth at SEQ ID NO: 102. In some embodiments, the GB1 domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence set forth in SEQ ID NO: 137. In some embodiments, a GB1 domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to an amino acid sequences set forth in SEQ ID NO: 137. In some embodiments, a GB1 domain of a prime editor comprises an amino acid sequence set forth at SEQ ID NO: 137.

In some embodiments, a prime editor comprising GB1 domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID NOs: 125-128. In some embodiments, a prime editor comprising GB1 domain comprises an amino acid sequence that has no more than 1, 2, 3, 4, 5, 6, 7, 8, 9, 10, 11, 12, 13, 14, 15, 16, 17, 18, 19, 20, 25, 30, 35, 40, 45, or 50 differences e.g., mutations e.g., deletions or substitutions compared to any of the amino acid sequences set forth in SEQ ID NO: 125-128. In some embodiments, a prime editor comprising GB1 domain of a prime editor comprises an amino acid sequence that is selected from the group consisting of: SEQ ID NOs: 125-128.

In some embodiments, a GB1 domain may be a basic GB1 (bGB1) domain. In some embodiments, a bGB1 domain may increase the solubility of a prime editor in vitro, relative to a prime editor comprising a GB1 domain. In some embodiments, a bGB1 domain may increase the solubility of a prime editor in vivo, relative to a prime editor comprising a GB1 domain. The increase in solubility of a prime editor comprising a bGB1 domain relative to a prime editor comprising a GB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in solubility of a prime editor comprising a bGB1 domain relative to a prime editor comprising a GB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in solubility of a prime editor comprising a bGB1 domain relative to a prime editor comprising a GB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in solubility of a prime editor comprising a bGB1 domain relative to a prime editor comprising a GB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a bGB1 domain may increase the expression level of a prime editor in vitro, relative to a prime editor comprising a GB1 domain. In some embodiments, a bGB1 domain may increase the expression level of a prime editor in vivo, relative to a prime editor comprising a GB1 domain. The increase in expression level of a prime editor comprising a bGB1 domain relative to a prime editor comprising a GB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in expression level of a prime editor comprising a bGB1 domain relative to a prime editor comprising a GB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in expression level of a prime editor comprising a bGB1 domain relative to a prime editor comprising a GB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in expression level of a prime editor comprising a bGB1 domain relative to a prime editor comprising a GB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold. The increase in the prime editing efficiency, DNA polymerase activity, DNA-binding activity, or DNA endonuclease activity of a prime editor comprising a bGB1 domain relative to a prime editor comprising a GB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%.

In some embodiments, a prime editor comprising a bGB1 domain comprises increased prime editing efficiency, DNA polymerase activity, DNA-binding activity, or DNA endonuclease activity relative to a prime editor comprising a GB1 domain. In some embodiments, the increase in the prime editing efficiency, DNA polymerase activity, DNA-binding activity, or DNA endonuclease activity of a prime editor comprising a bGB1 domain relative to a prime editor comprising a GB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in the prime editing efficiency, DNA polymerase activity, DNA-binding activity, or DNA endonuclease activity of a prime editor comprising a bGB1 domain relative to a prime editor comprising a GB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in the prime editing efficiency, DNA polymerase activity, DNA-binding activity, or DNA endonuclease activity of a prime editor comprising a bGB1 domain relative to a prime editor comprising a GB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a bGB1 domain may increase the solubility of a prime editor in vitro, relative to a prime editor lacking the bGB1 domain. In some embodiments, a bGB1 domain may increase the solubility of a prime editor in vivo, relative to a prime editor lacking the bGB1 domain. The increase in solubility of a prime editor comprising a bGB1 domain relative to a prime editor lacking the bGB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in solubility of a prime editor comprising a bGB1 domain relative to a prime editor lacking the bGB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in solubility of a prime editor comprising a bGB1 domain relative to a prime editor lacking the bGB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in solubility of a prime editor comprising a bGB1 domain relative to a prime editor lacking the bGB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a bGB1 domain may increase the expression level of a prime editor in vitro, relative to a prime editor lacking the bGB1 domain. In some embodiments, a bGB1 domain may increase the expression level of a prime editor in vivo, relative to a prime editor lacking the bGB1 domain. The increase in expression level of a prime editor comprising a bGB1 domain relative to a prime editor lacking the bGB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in expression level of a prime editor comprising a bGB1 domain relative to a prime editor lacking the bGB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in expression level of a prime editor comprising a bGB1 domain relative to a prime editor lacking the bGB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in expression level of a prime editor comprising a bGB1 domain relative to a prime editor lacking the bGB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, a prime editor comprising a bGB1 domain may have increased prime editing efficiency, DNA polymerase activity, DNA-binding activity, or DNA endonuclease activity relative to a prime editor lacking a bGB1 domain. The increase in the prime editing efficiency, DNA polymerase activity, DNA-binding activity, or DNA endonuclease activity of a prime editor comprising a bGB1 domain relative to a prime editor lacking the bGB1 domain, in some case, may be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200%. In some embodiments, the increase in the prime editing efficiency, DNA polymerase activity, DNA-binding activity, or DNA endonuclease activity of a prime editor comprising a bGB1 domain relative to a prime editor lacking the bGB1 domain may be from 10 to 20%, from 15 to 25%, from 20 to 30%, from 25 to 35%, from 30 to 40%, from 35 to 45%, from 40 to 50%, from 45 to 55%, from 50 to 60%, from 55 to 65%, from 60 to 70%, from 65 to 75%, from 70 to 80%, from 75 to 85%, from 80 to 90%, from 85 to 95%, from 90 to 100%, from 95 to 105%, from 100 to 110%, from 105 to 115%, from 110 to 120%, from 115 to 125%, from 120 to 130%, from 125 to 135%, from 130 to 140%, from 135 to 145%, from 140 to 150%, from 145 to 155%, from 150 to 160%, from 155 to 165%, from 160 to 170%, from 165 to 175%, from 170 to 180%, from 175 to 185%, from 180 to 190%, from 185 to 195%, or from 190 to 200%. The increase in the prime editing efficiency, DNA polymerase activity, DNA-binding activity, or DNA endonuclease activity of a prime editor comprising a bGB1 domain relative to a prime editor lacking the bGB1 domain, in some case, may be at least about at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 15-fold, at least about 20-fold, at least about 25-fold, at least about 30-fold, at least about 35-fold, at least about 40-fold, at least about 45-fold, or at least about 50-fold. In some embodiments, the increase in the prime editing efficiency, DNA polymerase activity, DNA-binding activity, or DNA endonuclease activity of a prime editor comprising a bGB1 domain relative to a prime editor lacking the bGB1 domain may be from 1-fold to 2-fold, from 1.5-fold to 2.5-fold, from 2-fold to 3-fold, from 2.5-fold to 3.5-fold, from 3-fold to 4-fold, from 3.5-fold to 4.5-fold, from 4-fold to 5-fold, from 4.5-fold to 5.5-fold, from 5-fold to 6-fold, from 5.5-fold to 6.5-fold, from 6-fold to 7-fold, from 6.5-fold to 7.5-fold, from 7-fold to 8-fold, from 7.5-fold to 8.5-fold, from 8-fold to 9-fold, from 8.5-fold to 9.5-fold, from 9-fold to 10-fold, from 9.5-fold to 20-fold, from 15-fold to 25-fold, from 20-fold to 30-fold, from 25-fold to 35-fold, from 30-fold to 40-fold, from 35-fold to 45-fold, or from 40-fold to 50-fold.

In some embodiments, the bGB1 domain comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, identical to an amino acid sequence set forth in SEQ ID NO: 137. In some embodiments, a bGB1 domain of a prime editor comprises an amino acid sequence set forth at SEQ ID NO: 137.

In some embodiments, a bGB1 domain may comprise asparagine at position 22 of SEQ ID NO: 102, arginine at position 36 of SEQ ID NO: 102, or lysine at position 42 of SEQ ID NO: 102. In some embodiments, a bGB1 domain may comprise asparagine at position 22 of SEQ ID NO: 102. In some embodiments, a bGB1 domain may comprise arginine at position 36 of SEQ ID NO: 102. In some embodiments, a bGB1 domain may comprise lysine at position 42 of SEQ ID NO: 102. In other cases, a bGB1 domain may comprise asparagine at position 22 of SEQ ID NO: 102, arginine at position 36 of SEQ ID NO: 102, and lysine at position 42 of SEQ ID NO: 102. In some embodiments, a bGB1 domain may have an isoelectric point (pI) of about 8. In some embodiments, a bGB1 domain may have an isoelectric point (pI) of about 8.1 In some embodiments, a bGB1 domain may have an isoelectric point (pI) of about 8.2. In some embodiments, a bGB1 domain may have an isoelectric point (pI) of about 8.3. In some embodiments, a bGB1 domain may have an isoelectric point (pI) of about 8.4. In some embodiments, a bGB1 domain may have an isoelectric point (pI) of about 8.5. In some embodiments, a bGB1 domain may have an isoelectric point (pI) of about 8.6. In some embodiments, a bGB1 domain may have an isoelectric point (pI) of about 8.7. In some embodiments, a bGB1 domain may have an isoelectric point (pI) of about 8.8. In some embodiments, a bGB1 domain may have an isoelectric point (pI) of about 8.9. In some embodiments, a bGB1 domain may have an isoelectric point (pI) of 8.67.

DNA Binding Domain

In certain aspects, the prime editors provided herein comprises a polypeptide domain having DNA binding activity (e.g., a DNA binding domain). In certain aspects, the prime editors provided herein comprise a DNA binding domain comprising an amino acid sequence at least 85% identical (e.g., at least 86%, at least 87%, at least 88%, at least 89%, at least 90%, at least 91%, at least 92%, at least 93%, at least 94%, at least 95%, at least 96%, at least 97%, at least 98%, at least 99% identical, or 100% identical) to any one of the sequences set forth in SEQ ID NO: 138-146, 494, 858, 1100 (Table 8). In some embodiments, the DNA-binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions or substitutions compared to any of the amino acid sequences set forth in SEQ ID NO: 138-146, 494, 858, or 1100. In some embodiments, the prime editors provided herein comprises a DNA binding domain comprising an amino acid sequence that does not a have a N-terminus methionine. In some embodiments, the prime editors provided herein comprises a DNA binding domain comprising an amino acid sequence comprising a N-terminus methionine. In some embodiments, the amino acid sequence of a DNA binding domain may be N-terminally modified by one or more processing enzymes, e.g., by Methionine aminopeptidases (MAP).

In certain aspects, the prime editors provided herein comprise a DNA binding domain comprising an amino acid sequence at least 85% identical (e.g., at least 86%, at least 87%, at least 88%, at least 89%, at least 90%, at least 91%, at least 92%, at least 93%, at least 94%, at least 95%, at least 96%, at least 97%, at least 98%, at least 99% identical, or 100% identical) to any one of the sequences set forth in SEQ ID NO: 495-503 (Table 8). In some embodiments, the DNA-binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions or substitutions compared to any of the amino acid sequences set forth in SEQ ID NO: 495-503.

In some embodiments, the DNA binding domain comprises a nuclease activity, for example, RNA-guided DNA endonuclease activity of a Cas polypeptide. In some embodiments, the DNA binding domain comprises a nuclease domain or nuclease activity. In some embodiments, DNA binding domain comprises a nickase, or a fully active nuclease. As used herein, the term “nickase” refers to a nuclease capable of cleaving only one strand of a double-stranded DNA target. In some embodiments, the prime editor comprises a DNA binding domain that is an inactive nuclease. In some embodiments, the DNA-binding domain is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene.

In some embodiments, the polypeptide domain comprises a DNA binding domain. In some embodiments, the polypeptide domain comprises a DNA endonuclease domain. In some embodiments, a prime editor comprises a DNA binding domain and a DNA endonuclease domain. In some embodiments, the DNA-binding domain and the DNA endonuclease domain may comprise the same amino acid sequence. In one case, the DNA-binding domain and the DNA endonuclease domain may comprise overlapping amino acids. In some embodiments, the DNA-binding domain and the DNA endonuclease domain may comprise non-overlapping amino acids, e.g., the DNA-binding domain and the DNA endonuclease domain may comprise two independent amino acid sequences. In some embodiments, a prime editor may comprise more than one DNA-binding domain. In some embodiments, a prime editor may comprise more than one DNA endonuclease domain.

In some embodiments, a prime editor may comprise DNA-binding activity or a DNA endonuclease activity. In some embodiments, a prime editor may comprise a DNA-binding activity or a DNA endonuclease activity. In some embodiments, a prime editor may comprise a DNA endonuclease activity. In some embodiments, a prime editor may comprise a DNA-binding activity and a DNA endonuclease activity.

In some embodiments, a prime editor comprises an endonuclease domain having single strand DNA cleavage activity. For example, the endonuclease domain may be a Fokl nuclease domain. In some embodiments, a prime editor comprises an endonuclease having with modified or reduced nuclease activity as compared to a wild type endonuclease domain. For example, the endonuclease domain may comprise one or more amino acid substitutions as compared to a wild type endonuclease domain. As a result, the endonuclease domain may have single strand DNA cleavage activity (i.e., a nickase) when contacted with a double stranded DNA sequence. In some instances, the endonuclease domain may comprise one or more amino acid substitutions that abolish the nuclease activity as compared to a wild type endonuclease.

The DNA-binding domain of a prime editor, in some embodiments, may comprise a Clustered Regularly Interspaced Short Palindromic Repeats (CRISPR) Associated (Cas) polypeptide, a zinc-finger nuclease (ZFN) and/or a transcription activator-like effector nucleases (TALEN).

### Cas Protein

In some embodiments, the DNA-binding domain of a prime editor may comprise a Cas protein. A Cas protein may be a Class 1 or a Class 2 Cas protein. A Cas protein may be a type I, type II, type III, type IV, type V Cas protein, or type VI Cas protein. A Cas protein may comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein domain comprises a guide nucleic acid recognition and/or a binding domain that may interact with a guide nucleic acid, and one or more nuclease domains that comprise catalytic activity for nucleic acid cleavage. In some embodiments, a Cas protein may comprise a chimeric Cas protein that is fused to other proteins or polypeptides. A Cas protein may comprise be a chimera of various Cas proteins, for example, comprising domains from different Cas proteins.

In some embodiments, a prime editor comprises a DNA binding domain that is a Cas polypeptide or a mutant, variant, or functional fragment thereof. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas1Od, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), Cas D, and homologues, modified or engineered variants, mutants, and/or functional fragments thereof.

A Cas polypeptide may be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some aspects, the organism is Staphylococcus lugdunensis (S. lugdunensis).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein may be a wildtype or a modified form of a Cas protein. A Cas protein can be an active variant, inactive variant, or fragment of a wild type or modified Cas protein. A Cas protein as described herein may comprise an amino acid change such as a deletion, insertion, substitution, variant, mutation, fusion, chimera, or any combination thereof relative to a wild-type version of the Cas protein. A Cas protein may be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein. A Cas protein may be a polypeptide with at most about 5%, 10%, 20%, 30%, 40%, 50%, 60%, 70%, 80%, 90%, 100% sequence identity and/or sequence similarity to a wild type exemplary Cas protein. A Cas protein comprise an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., deletions or substitutions compared to a wild type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein may comprise one or more nuclease domains, such as DNase domains. For example, a Cas9 protein may comprise a RuvC-like nuclease domain and/or an HNH-like nuclease domain. The RuvC and HNH domains may each cut a different strand of double-stranded DNA to make a double-stranded break in the DNA. A Cas protein may comprise only one nuclease domain (e.g., Cpf1 comprises RuvC domain but lacks HNH domain).

A Cas protein may comprise an ammo acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein. A Cas protein comprise an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 differences e.g., mutations e.g., deletions or substitutions compared to a a nuclease domain (e.g., RuvC domain, HNH domain) of a wild-type Cas protein.

A Cas protein may be modified to optimize regulation of gene expression. A Cas protein may be modified to increase or decrease nucleic acid binding affinity, nucleic acid binding specificity, and/or enzymatic activity. Cas proteins may also be modified to change any other activity or property of the protein, such as stability. For example, one or more nuclease domains of the Cas protein may be modified, deleted, or inactivated, or a Cas protein may be truncated to remove domains that are not essential for the function of the protein or to optimize (e g, enhance or reduce) the activity of the Cas protein for regulating gene expression.

A Cas protein may be a fusion protein. For example, a Cas protein may be fused to a cleavage domain, an epigenetic modification domain, a transcriptional activation domain, or a transcriptional repressor domain. A Cas protein may also be fused to a heterologous polypeptide providing increased or decreased stability. The fused domain or heterologous polypeptide may be located at the N-terminus, the C-terminus, or internally within the Cas protein.

A Cas protein may be provided in any form. For example, a Cas protein may be provided in the form of a protein, such as a Cas protein alone or complexed with a guide nucleic acid. A Cas protein may be provided in the form of a nucleic acid encoding the Cas protein, such as an RNA (e.g., messenger RNA (mRNA)) or DNA. The nucleic acid encoding the Cas protein may be codon optimized for efficient translation into protein in a particular cell or organism.

Nucleic acids encoding Cas proteins may be stably integrated in the genome of the cell. Nucleic acids encoding Cas proteins may be operably linked to a promoter active in the cell. Nucleic acids encoding Cas proteins may be operably linked to a promoter in an expression construct. Expression constructs may include any nucleic acid constructs capable of directing expression of a gene or other nucleic acid sequence of interest (e.g., a Cas gene) and which may transfer such a nucleic acid sequence of interest to a target cell. In some embodiments, the Cas molecule or Cas domain comprises a responsive intein. In some embodiments, a DNA binding domain may comprise a split Cas protein, e.g., a split Cas9. In some embodiments, a split refers to division into two or more fragments. In some embodiments, a split Cas9 protein may include an active nuclease, a nickase, and a nuclease-null Cas9 protein.

In some embodiments, a split Cas9 reconstitutes a full-length Cas9 protein at least 20%, at least 30%, at least 40%, at least 50%, at least 60%, at least 70%, at least 75%, at least 80%, at least 85%, at least 90%, at least 95%, at least 96%, at least 97%, at least 98%, at least 99%, or at least 100% efficiency compared to a Cas9 that is not split.

A Cas protein may comprise a modified form of a wild type Cas protein. The modified form of the wild type Cas protein may comprise an amino acid change (e.g., deletion, insertion, or substitution) that reduces the nucleic acid-cleaving activity of the Cas protein. For example, the modified form of the Cas protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein may have no substantial nucleic acid-cleaving activity. When a Cas protein is a modified form that has no substantial nucleic acid-cleaving activity, it may be referred to as enzymatically inactive and/or “dead” (abbreviated by “d”). A dead Cas protein (e.g., dCas, dCas9) may bind to a target polynucleotide but may not cleave the target polynucleotide. In some aspects, a dead Cas protein is a dead Cas9 protein.

Enzymatically inactive can refer to a polypeptide that can bind to a nucleic acid sequence in a polynucleotide in a sequence-specific manner but may not cleave a target polynucleotide. An enzymatically inactive site-directed polypeptide may comprise an enzymatically inactive domain (e.g. nuclease domain). Enzymatically inactive can refer to no activity. Enzymatically inactive may refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type Cas9 activity).

One or a plurality of the nuclease domains (e.g., RuvC, HNH) of a Cas protein may be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, may generate a single-strand break at a CRISPR RNA (crRNA) recognition sequence within a double-stranded DNA but not a double-strand break. Such a nickase can cleave the complementary strand or the non-complementary strand but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein may have a reduced or no ability to cleave both strands of a double-stranded target DNA. An example of a mutation that may convert a Cas9 protein into a nickase is a D10A (aspartate to alanine at position 10 of Cas9 as set forth in SEQ ID NO: 138) mutation in the RuvC domain of Cas9 from S. pyogenes. A mutation corresponding to the H840A amino acid substitution (histidine to alanine at amino acid position 840 as set forth in SEQ ID NO: 138) in the HNH domain of Cas9 from S. pyogenes may convert the Cas9 into a nickase. An example of a mutation that may convert a Cas9 protein into a dead Cas9 is a D10A (aspartate to alanine at position 10 of Cas9) mutation in the RuvC domain and H840A (histidine to alanine at amino acid position 840) in the HNH domain of Cas9 from S. pyogenes.

A dead Cas protein may comprise one or more mutations relative to a wild-type version of the protein. The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type Cas protein. The mutation may result in one or more of the plurality of nucleic acid-cleaving domains retaining the ability to cleave the complementary strand of the target nucleic acid but reducing its ability to cleave the non-complementary strand of the target nucleic acid. The mutation may result in one or more of the plurality of nucleic acid-cleaving domains retaining the ability to cleave the non-complementary strand of the target nucleic acid but reducing its ability to cleave the complementary strand of the target nucleic acid. The mutation may result in one or more of the plurality of nucleic acid-cleaving domains lacking the ability to cleave the complementary strand and the non-complementary strand of the target nucleic acid. The residues to be mutated in a nuclease domain may correspond to one or more catalytic residues of the nuclease. For example, residues in the wild type exemplary S. pyogenes Cas9 polypeptide such as Asp10, His840, Asn854 and Asn856 may be mutated to inactivate one or more of the plurality of nucleic acid-cleaving domains (e.g., nuclease domains). The residues to be mutated in a nuclease domain of a Cas protein may correspond to residues Asp10, His840, Asn854 and Asn856 in the wild type S. pyogenes Cas9 polypeptide, for example, as determined by sequence and/or structural alignment.

As non-limiting examples, one or more of amino acid residues D10, G12, G17, E762, H840, N854, N863, H982, H983, A984, D986, and/or A987 in a SpCas9 as set forth in SEQ ID NO: 138, or corresponding amino acid residues in another Cas9 protein may be mutated. For example, a Cas9 protein variant may comprise one or more of D10A, G12A, G17A, E762A, H840A, N854A, N863A, H982A, H983A, A984A, and/or D986A amino acid substitutions as set forth in SEQ ID NO: 138 or corresponding mutations. Mutations other than alanine substitutions can be suitable.

A D10A mutation may be combined with one or more of H840A, N854A, or N856A mutations to produce a Cas protein substantially lacking DNA cleavage activity (e.g., a dead Cas9 protein). A H840A mutation may be combined with one or more of D10A, N854A, or N856A mutations to produce a site-directed polypeptide substantially lacking DNA cleavage activity. A N854A mutation may be combined with one or more of H840A, D10A, or N856A mutations to produce a site-directed polypeptide substantially lacking DNA cleavage activity. A N856A mutation may be combined with one or more of H840A, N854A, or D10A mutations to produce a site-directed polypeptide substantially lacking DNA cleavage activity.

In some embodiments, the DNA-binding domain comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain. In some embodiments, the Cas nickase is a Cas9 nickase comprising one or more mutation in the HNH domain that reduces or abolishes nuclease activity of the HNH domain. Sequences of exemplary Cas9 nickase variants and corresponding prime editors are provided in Table 8. In some embodiments, the Cas9 nickase comprises one or more of amino acid substitutions corresponding to the nickase mutations as provided in Table 8 when aligned against the corresponding reference nuclease Cas9 sequence in Table 8.

A Cas protein domain provided herein can be a nuclease active variant, nuclease inactive variant, a nickase, or a functional variant or functional fragment of a wild type Cas protein. A Cas protein domain provided herein can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to an exemplary Cas protein domain provided herein.

A Cas protein domain may be a fusion protein. For example, a Cas protein domain provided herein can be fused to a cleavage domain, an epigenetic modification domain, a transcriptional regulation domain, or a polymerase domain. A Cas domain protein can also be fused to a heterologous polypeptide providing increased or decreased stability. The fused domain or heterologous polypeptide can be located at the N-terminus, the C-terminus, or internally within the Cas protein domain.

As described herein, the Cas protein domain may be between 800 and 1500 amino acids in length, between 1400 and 900 amino acids in length, or at least 1000 and 1300 amino acids in length. The Cas9 protein domain may be at least 800 amino acids in length, at least 900 amino acids in length, at least 1000 amino acids in length, at least 1100 amino acids in length, or at least 1200 amino acids in length. In some embodiments, the Cas9 protein domain is 1057 amino acids in length. In some embodiments, the Cas protein domain is 1069 amino acids in length. In some embodiments, the Cas protein domain is 1369 amino acids in length.

In some embodiments, the Cas protein domain recognizes the PAM sequence “NGA,” wherein N is any nucleotide. In some embodiments, the Cas protein domain recognizes the PAM sequence “NGN,” wherein N is any nucleotide. In some embodiments, the Cas protein domain recognizes the PAM sequence “NRN,” wherein N is any nucleotide. In some embodiments, the Cas protein domain recognizes the PAM sequence “NNGRRT,” wherein N is any nucleotide. In some embodiments, the Cas protein domain recognizes the PAM sequence “NNGG,” wherein N is any nucleotide.

In some embodiments, a prime editor provided herein comprises a Cas protein domain that contains modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein domain. The specific PAM sequence required for Cas protein domain recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM.

In some embodiments, a prime editor comprises a DNA binding domain that has nickase activity to cleave a first strand of a double stranded target DNA sequence. In some cases, the prime editor may cleave a first stand of a double stranded target DNA sequence. In some cases, the first strand of a double stranded target DNA sequence cleavable a prime editor may comprise a PAM sequence. In one case, when the first strand of a double stranded target DNA sequence cleavable by a a prime editor comprises a PAM sequence, the second strand of the double stranded target DNA sequence may comprise a complement of the PAM sequence.

In some embodiments, a Cas protein domain comprises one or more nuclease domains. A Cas protein domain may comprise an amino acid sequence having at least about 50%, 60%, 70%, 80%, 90%, 91%, 92%, 93%, 94%, 95%, 96%, 97%, 98%, 99%, or 100% sequence identity to a nuclease domain of a wild-type Cas protein. In some embodiments, a Cas protein domain comprises a single nuclease domain

In some embodiments, a prime editor comprises a Cas protein domain that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity).

Exemplary Cas protein domains are shown in Table 8. In some embodiments, the Cas protein domain is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence provided in Table 8. In some embodiments, the Cas protein domain comprises an amino acid sequence identical to any one of the sequences set forth in SEQ ID NO: 138-146, 494, 858, 1100 (e.g., Table 8). It should be appreciated that for each of the variants provided, the Cas protein comprises one or more of the amino acid substitutions compared to a wild type Cas protein sequence, for example, the Cas9 as set forth in SEQ ID NO: 138 or SEQ ID NO: 858.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking substantial cleavage activity. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. Aureus. In some embodiments, the Cas9 protein is derived from a Cas9 protein from S. pyogenes or S. Aureus. For example, a S. pyogenes or S. aureus Cas9 protein lacking substantial cleavage activity.

In some instances, a Cas9 protein may comprise a wildtype Cas9 protein or a variant Cas9 protein, functional portion of any of these, fusion protein of any of these, or any combinations thereof. In some cases, a Cas9 polypeptide may comprise a wildtype Cas9 polypeptide. In some cases, a Cas9 polypeptide may comprise a variant Cas9 polypeptide.

In some embodiments, the DNA binding domain comprises a nuclease-active Cas domain, a Cas nickase (nCas) domain, e.g., nCas9 domain, or a nuclease-5 inactive Cas (dCas) domain, e.g., dCas9 domain. In some embodiments, the DNA binding domain comprises a Cas9 domain of Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i or a mutant, functional fragment, or variant thereof.

In some instances, a Cas9 polypeptide of a prime editor may comprise a nickase activity. In some cases, the Cas9 polypeptide may comprise a Cas9 nickase. In some cases, a wildtype Cas9 polypeptide may cleave both strands of a double stranded target DNA sequence. In some cases, a Cas9 nickase may cleave one strand of a double stranded target DNA sequence. In some cases, a Cas9 nickase may comprise a mutation in a wildtype Cas9 polypeptide. Such mutation may comprise any mutation described herein.

In some embodiments, the Cas9 protein domain recognizes a PAM sequence flanked by a spacer. In some embodiments, the spacer is on the 5′ end of the PAM sequence. In some embodiments, the spacer is on the 3′ end of the PAM sequence. In some embodiments, the spacer is 10, 11, 12, 13, 14, 15, 16, 17, 18, 19, 20, 21, 22, 23, 24, 25, 26, 27, 28, 29, or 30 nucleotides in length.

In some embodiments, a Cas9 protein domain of a prime editor may comprise an alanine-to-histidine substitution at 840th position (H840A), an aspartic acid-to-alanine substitution at the 10th position (D10A), or at the corresponding position of the wildtype Cas9 polypeptide. In some cases, a Cas9 polypeptide of a prime editor may comprise mutation H840A of a SpCas9 polypeptide. In some cases, a Cas9 polypeptide of a prime editor may comprise mutation H840A or at the corresponding position of the wildtype Cas9 polypeptide. In some cases, a Cas9 polypeptide of a prime editor may comprise mutation D10A, or at the corresponding position of the wildtype Cas9 polypeptide. In some cases, a Cas9 polypeptide of a prime editor may comprise mutation D10A of a wildtype SpCas9 polypeptide. In other cases, a Cas9 polypeptide of a prime editor may comprise H840A and D10A mutations, or at the corresponding position of the wildtype Cas9 polypeptide.

A wildtype Cas9 polypeptide may comprise a RuvC domain and an HNH domain. A Cas9 polypeptide with a functional RuvC domain and a functional HNH domain may cleave both strands of a double stranded target DNA sequence. A Cas9 polypeptide with only one functional RuvC domain or one functional HNH domain may cleave one strand of a double stranded target DNA sequence. A Cas9 polypeptide without one functional RuvC domain and one functional HNH domain may not cleave any strand of a double stranded target DNA sequence.

In some embodiments, a Cas9 polypeptide may comprise a RuvC domain. In some cases, a mutation in the RuvC domain of a Cas9 polypeptide may comprise mutation D10A. A mutation in the RuvC domain of a Cas9 polypeptide, in some cases, may comprise mutation D10A or structural equivalent thereof of a wildtype SpCas9 polypeptide. In other cases, a mutation in the RuvC domain of a Cas9 polypeptide, in some cases, may comprise mutation H983A, D986A, or E762A. A Cas9 polypeptide comprising a wildtype RuvC domain may cleave a second strand of a double stranded target DNA sequence. Such a second strand of a double stranded target DNA sequence, in some cases, may not comprise a PAM sequence. In some cases, a second strand of a double stranded target DNA sequence, cleavable by a Cas9 polypeptide comprising a wildtype RuvC domain, may comprise complement of a PAM sequence. A Cas9 polypeptide comprising a mutation in the RuvC domain may not cleave the second strand of a double stranded target DNA sequence.

In some embodiments, a Cas9 polypeptide may comprise an HNH domain. In some cases, a mutation in the HNH domain of a Cas9 polypeptide may comprise mutation H840A. A mutation in the HNH domain of a Cas9 polypeptide, in some cases, may comprise mutation H840A or structural equivalent thereof of a wildtype SpCas9 polypeptide. In other cases, a mutation in the HNH domain of a Cas9 polypeptide, in some cases, may comprise mutation N863A. A Cas9 polypeptide comprising a wildtype HNH domain may cleave a first strand of a double stranded target DNA sequence. Such a first strand of a double stranded target DNA sequence, in some cases, may comprise a PAM sequence. In some cases, a second strand of a double stranded target DNA sequence, cleavable by a Cas9 polypeptide comprising a wildtype HNH domain, may comprise complement of a PAM sequence. A Cas9 polypeptide comprising a mutation in the HNH domain may not cleave the first strand of a double stranded target DNA sequence. In certain instances, the Cas9 polypeptide in a prime editor is a Cas9 nickase comprising a mutation in the HNH domain which inactivates the HNH nuclease activity. For example, a prime editor may comprise a Cas9 nickase that comprises a H840X mutation and/or a N863X mutation, wherein X is any amino acid other than the wild type amino acid. In certain embodiments, the Cas9 nickase comprises a H840A or N863A or a combination thereof. In some instances, the Cas9 polypeptide comprises a RuvC domain. In some embodiments, the Cas9 polypeptide may be a nickase that comprises a RuvC domain and not an HNH domain.

In some embodiments, a Cas9 protein may comprise a Cas9 protein from Streptococcus pyogenes (Sp), Staphylococcus aureus (Sa), Streptococcus canis (Sc), Streptococcus thermophilus (St), Neisseria meningitidis (Nm), Campylobacter jejuni (Cj), Francisella novicida (Fn), or Treponema denticola (Td). In some cases, a Cas9 polypeptide may comprise a SpCas9 polypeptide. In some cases, a Cas9 polypeptide may comprise a SaCas9 polypeptide. In some cases, a Cas9 polypeptide may comprise a ScCas9 polypeptide. In some cases, a Cas9 polypeptide may comprise a StCas9 polypeptide. In some cases, a Cas9 polypeptide may comprise a NmCas9 polypeptide. In some cases, a Cas9 polypeptide may comprise a CjCas9 polypeptide. In some cases, a Cas9 polypeptide may comprise a FnCas9 polypeptide. In some cases, a Cas9 polypeptide may comprise a TdCas9 polypeptide.

An exemplary Streptococcus pyogenes Cas9 (SpCas9) amino acid sequence is provided below:

(SEQ ID NO: 138)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

An exemplary Staphylococcus aureus (SaCas9) amino acid sequence is provided below:

(SEQ ID NO: 140)
MKRNYILGLDIGITSVGYGIIDYETRDVIDAGVRLFKEANVENNEGRRS
KRGARRLKRRRRHRIQRVKKLLFDYNLLTDHSELSGINPYEARVKGLSQ
KLSEEEFSAALLHLAKRRGVHNVNEVEEDTGNELSTKEQISRNSKALEE
KYVAELQLERLKKDGEVRGSINRFKTSDYVKEAKQLLKVQKAYHQLDQS
FIDTYIDLLETRRTYYEGPGEGSPFGWKDIKEWYEMLMGHCTYFPEELR
SVKYAYNADLYNALNDLNNLVITRDENEKLEYYEKFQIIENVFKQKKKP
TLKQIAKEILVNEEDIKGYRVTSTGKPEFTNLKVYHDIKDITARKEIIE
NAELLDQIAKILTIYQSSEDIQEELTNLNSELTQEEIEQISNLKGYTGT
HNLSLKAINLILDELWHTNDNQIAIFNRLKLVPKKVDLSQQKEIPTTLV
DDFILSPVVKRSFIQSIKVINAIIKKYGLPNDIIIELAREKNSKDAQKM
INEMQKRNRQTNERIEEIIRTTGKENAKYLIEKIKLHDMQEGKCLYSLE
AIPLEDLLNNPFNYEVDHIIPRSVSFDNSFNNKVLVKQEEASKKGNRTP
FQYLSSSDSKISYETFKKHILNLAKGKGRISKTKKEYLLEERDINRFSV
QKDFINRNLVDTRYATRGLMNLLRSYFRVNNLDVKVKSINGGFTSFLRR
KWKFKKERNKGYKHHAEDALIIANADFIFKEWKKLDKAKKVMENQMFEE
KQAESMPEIETEQEYKEIFITPHQIKHIKDFKDYKYSHRVDKKPNRELI
NDTLYSTRKDDKGNTLIVNNLNGLYDKDNDKLKKLINKSPEKLLMYHHD
PQTYQKLKLIMEQYGDEKNPLYKYYEETGNYLTKYSKKDNGPVIKKIKY
YGNKLNAHLDITDDYPNSRNKVVKLSLKPYRFDVYLDNGVYKFVTVKNL
DVIKKENYYEVNSKCYEEAKKLKKISNQAEFIASFYNNDLIKINGELYR
VIGVNNDLLNRIEVNMIDITYREYLENMNDKRPPRIIKTIASKTQSIKK
YSTDILGNLYEVKSKKHPQIIKK

An exemplary Staphylococcus aureus (SaCas9) amino acid sequence is provided below:

(SEQ ID NO: 858)
MKRNYILGLDIGITSVGYGIIDYETRDVIDAGVRLFKEANVENNEGRRS
KRGARRLKRRRRHRIQRVKKLLFDYNLLTDHSELSGINPYEARVKGLSQ
KLSEEEFSAALLHLAKRRGVHNVNEVEEDTGNELSTKEQISRNSKALEE
KYVAELQLERLKKDGEVRGSINRFKTSDYVKEAKQLLKVQKAYHQLDQS
FIDTYIDLLETRRTYYEGPGEGSPFGWKDIKEWYEMLMGHCTYFPEELR
SVKYAYNADLYNALNDLNNLVITRDENEKLEYYEKFQIIENVFKQKKKP
TLKQIAKEILVNEEDIKGYRVTSTGKPEFTNLKVYHDIKDITARKEIIE
NAELLDQIAKILTIYQSSEDIQEELTNLNSELTQEEIEQISNLKGYTGT
HNLSLKAINLILDELWHTNDNQIAIFNRLKLVPKKVDLSQQKEIPTTLV
DDFILSPVVKRSFIQSIKVINAIIKKYGLPNDIIIELAREKNSKDAQKM
INEMQKRNRQTNERIEEIIRTTGKENAKYLIEKIKLHDMQEGKCLYSLE
AIPLEDLLNNPFNYEVDHIIPRSVSFDNSFNNKVLVKQEENSKKGNRTP
FQYLSSSDSKISYETFKKHILNLAKGKGRISKTKKEYLLEERDINRFSV
QKDFINRNLVDTRYATRGLMNLLRSYFRVNNLDVKVKSINGGFTSFLRR
KWKFKKERNKGYKHHAEDALIIANADFIFKEWKKLDKAKKVMENQMFEE
KQAESMPEIETEQEYKEIFITPHQIKHIKDFKDYKYSHRVDKKPNRELI
NDTLYSTRKDDKGNTLIVNNLNGLYDKDNDKLKKLINKSPEKLLMYHHD
PQTYQKLKLIMEQYGDEKNPLYKYYEETGNYLTKYSKKDNGPVIKKIKY
YGNKLNAHLDITDDYPNSRNKVVKLSLKPYRFDVYLDNGVYKFVTVKNL
DVIKKENYYEVNSKCYEEAKKLKKISNQAEFIASFYNNDLIKINGELYR
VIGVNNDLLNRIEVNMIDITYREYLENMNDKRPPRIIKTIASKTQSIKK
YSTDILGNLYEVKSKKHPQIIKKG

In some embodiments, a prime editor comprises a Cas9 protein from Staphylococcus lugdunensis (Slu Cas9). An exemplary amino acid sequence of a Staphylococcus lugdunensis (Slu Cas9) is provided in SEQ ID NO: 139.

(SEQ ID NO: 139)
MNQKFILGLDIGITSVGYGLIDYETKNIIDAGVRLFPEANVENNEGRRS
KRGSRRLKRRRIHRLERVKKLLEDYNLLDQSQIPQSTNPYAIRVKGLSE
ALSKDELVIALLHIAKRRGIHKIDVIDSNDDVGNELSTKEQLNKNSKLL
KDKFVCQIQLERMNEGQVRGEKNRFKTADIIKEIIQLLNVQKNFHQLDE
NFINKYIELVEMRREYFEGPGKGSPYGWEGDPKAWYETLMGHCTYFPDE
LRSVKYAYSADLFNALNDLNNLVIQRDGLSKLEYHEKYHIIENVFKQKK
KPTLKQIANEINVNPEDIKGYRITKSGKPQFTEFKLYHDLKSVLFDQSI
LENEDVLDQIAEILTIYQDKDSIKSKLTELDILLNEEDKENIAQLTGYT
GTHRLSLKCIRLVLEEQWYSSRNQMEIFTHLNIKPKKINLTAANKIPKA
MIDEFILSPVVKRTFGQAINLINKIIEKYGVPEDIIIELARENNSKDKQ
KFINEMQKKNENTRKRINEIIGKYGNQNAKRLVEKIRLHDEQEGKCLYS
LESIPLEDLLNNPNHYEVDHIIPRSVSFDNSYHNKVLVKQSENSKKSNL
TPYQYFNSGKSKLSYNQFKQHILNLSKSQDRISKKKKEYLLEERDINKF
EVQKEFINRNLVDTRYATRELTNYLKAYFSANNMNVKVKTINGSFTDYL
RKVWKFKKERNHGYKHHAEDALIIANADFLFKENKKLKAVNSVLEKPEI
ESKQLDIQVDSEDNYSEMFIIPKQVQDIKDFRNFKYSHRVDKKPNRQLI
NDTLYSTRKKDNSTYIVQTIKDIYAKDNTTLKKQFDKSPEKFLMYQHDP
RTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLD
VLKKDNYYYIPEQKYDKLKLGKAIDKNAKFIASFYKNDLIKLDGEIYKI
IGVNSDTRNMIELDLPDIRYKEYCELNNIKGEPRIKKTIGKKVNSIEKL
TTDVLGNVFTNTQYTKPQLLFKRGN

The Slu Cas9 may comprise one or more mutations that modifies or reduces its nuclease activity. For example, the Slu Cas9 may comprise a mutation in a HNH domain, resulting in a Slu Cas9 nickase. In some embodiments a sluCas9 recognizes a “NNGG” PAM.

In some embodiments, a Cas9 is a modified Cas9; e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified sluCas9 shows increased editing efficiency and/or specificity relative to a sluCas9 that is not modified. In some embodiments, a modified Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in specificity compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in cleavage activity compared to a Cas9 that is not modified. In some embodiments, a Cas9, e.g., a sRGN shows ability to cleave a 5′-NNGG-3′ PAM-containing target.

Cas9 polypeptides and variants are also contemplated. In some embodiments, the N-terminal methionine is removed from a Cas protein domain provided herein. In some embodiments, a Cas9 polypeptide can comprise mutation A61R, L111R, D1135V, R221K, A262T, R324L, N394K, S4091, S4091, E427G, E480K, M495V, N497A, Y515N, K526E, F539S, E543D, R654L, R661A, R661L, R691A, N692A, M694A, M6941, Q695A, H698A, R753G, M7631, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, or any combinations thereof of a wildtype SpCas9 polypeptide.

In some embodiments, a Cas9 polypeptide of a prime editor may comprise a Cas9 polypeptide other than a SpCas9 polypeptide. In some cases, a SaCas9 polypeptide of a prime editor may comprise a wildtype SaCas9 polypeptide. In some cases, a SaCas9 polypeptide of a prime editor may comprise a variant SaCas9 polypeptide. In some cases, a SaCas9 polypeptide may comprise mutation E782K, N968K, or R1015H. In some cases, a FnCas9 polypeptide may comprise a wildtype FnCas9 polypeptide. In some cases, a FnCas9 polypeptide may comprise a variant FnCas9 polypeptide. In some cases, a FnCas9 polypeptide may comprise mutation E1369R, E1449H, or R1556A. In some cases, a StCas9 may comprise a Stl Cas9 or a St3 Cas9 polypeptide. In some cases, a ScCas9 polypeptide may comprise a wildtype ScCas9 polypeptide. In some cases, a ScCas9 polypeptide may comprise a variant ScCas9 polypeptide. In some cases, a ScCas9 polypeptide may comprise mutation 1367K, G368D, 1369K, H371L, T375S, T376G, or T1227K.

In some instances, the DNA binding domain of a prime editor, e.g., Cas9 domain, may be configured to cleave the first strand of a double stranded target DNA sequence at a cleavage site upstream of a PAM sequence. In some cases, the second polypeptide of a prime editor may be configured to cleave the first strand of a double stranded target DNA sequence at a cleavage site 5′ of a PAM sequence. In some cases, the cleavage site is at least 1, at least 2, at least 3, at least 4, at least 5, at least 6, at least 7, at least 8, at least 9 nucleotides upstream or 5′ of the PAM sequence. In some cases, the cleavage site is at least 1 nucleotide upstream or 5′ of the PAM sequence. In some cases, the cleavage site is at least 2 nucleotides upstream or 5′ of the PAM sequence. In some cases, the cleavage site is at least 3 nucleotides upstream or 5′ of the PAM sequence. In some cases, the cleavage site is at least 4 nucleotides upstream or 5′ of the PAM sequence. In some cases, the cleavage site is at least 5 nucleotides upstream or 5′ of the PAM sequence. In some cases, the cleavage site is at least 6 nucleotides upstream or 5′ of the PAM sequence. In some cases, the cleavage site is at least 7 nucleotides upstream or 5′ of the PAM sequence. In some cases, the cleavage site is at least 8 nucleotides upstream or 5′ of the PAM sequence. In some cases, the cleavage site is at least 9 nucleotides upstream or 5′ of the PAM sequence. In some cases, the cleavage site is from 10 to 20 nucleotides, from 15 to 25 nucleotides, from 20 to 30 nucleotides, from 25 to 35 nucleotides, from 30 to 40 nucleotides, from 35 to 45, or from 45 to 50 nucleotides upstream or 5′ of the PAM sequence.

In some embodiments, a PAM sequence in a first strand of a double stranded target DNA sequence may comprise NGG, wherein N is A, C, T, or G and applied to other PAM sequence described herein. In some cases, a prime editor may cleave a double stranded target DNA sequence comprising NGG as a PAM sequence in the first strand of the double stranded target DNA sequence. In some cases, a prime editor comprising a second polypeptide may cleave a double stranded target DNA sequence comprising NGG as a PAM sequence in the first strand of the double stranded target DNA sequence. In some cases, a prime editor comprising a Cas9 polypeptide as a second polypeptide may cleave a double stranded target DNA sequence comprising NGG as a PAM sequence in the first strand of the double stranded target DNA sequence. In some cases, a prime editor comprising a wildtype SpCas9 polypeptide as a second polypeptide may cleave a double stranded target DNA sequence comprising NGG as a PAM sequence in the first strand of the double stranded target DNA sequence. In some cases, a SpCas9 polypeptide may be modified to alter the PAM specificity. In some cases, a Cas9 polypeptide comprising mutations D1135V, R1335Q, and T1337R may cleave a double stranded target DNA sequence comprising NGAN or NGNG as a PAM sequence in the first strand of the double stranded target DNA sequence. In some cases, a SpCas9 polypeptide comprising mutations D1135E, R1335Q, and T1337R may cleave a double stranded target DNA sequence comprising NGAG as a PAM sequence in the first strand of the double stranded target DNA sequence. In some cases, a SpCas9 polypeptide comprising mutations D1135V, G1218R, R1335E, and T1337R may cleave a double stranded target DNA sequence comprising NGCG as a PAM sequence in the first strand of the double stranded target DNA sequence. In some cases, a SpCas9 polypeptide comprising mutation D1135E may have enhanced specificity to NGG when compared to a wildtype SpCas9 polypeptide. In some cases, a SaCas9 may recognize NGRRT or NGRRN as a PAM sequence. In some cases, a NmCas9 may recognize NNNNGATT as a PAM sequence. In some cases, a StCas9 may recognize NNAGAAW as a PAM sequence. In some cases, a NmCas9 may recognize NNNNGATT as a PAM sequence. In some cases, a TdCas9 may recognize NAAAAC as a PAM sequence.

An exemplary sequence of a Cas9 recognizing a NG PAM sequence is provided below:

(SEQ ID NO: 1100)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESIRPK

In some embodiments, a Cas protein domain provided herein comprises a Cas fragment that is a functional fragment of a Cas protein domain provided herein that retains one or more Cas activities. In some embodiments, the Cas fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a Cas9 domain may comprise an amino acid sequence comprising at least 85% identical (e.g., at least 86%, at least 87%, at least 88%, at least 89%, at least 90%, at least 91%, at least 92%, at least 93%, at least 94%, at least 95%, at least 96%, at least 97%, at least 98%, at least 99% identical, or 100% identical) to any one of the sequences set forth in: SEQ ID NOs:138-146 or 1100. In some embodiments, the Cas9 domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions or substitutions compared to any of the amino acid sequences set forth in SEQ ID NO: 138-146, 858, or 1100.

In some embodiments, a prime editor comprises a Cas protein domain that comprises a circular permutant Cas variant. For example, a Cas protein domain of a prime editor may be engineered such that the N-terminus and the C-terminus of a Cas protein domain (e.g., a Cas protein domain provided herein) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein domain may have the following structure: N-terminus-[original C-terminus]-[optional linker]-[original N-terminus]-C-terminus.

In some embodiments, the circular permutant can be formed by linking a C-terminal fragment of a Cas to an N-terminal fragment of a Cas, either directly or by using a linker, such as an amino acid linker. In some embodiments, The C-terminal fragment may correspond to the C-terminal 95% or more of the amino acids of a Cas protein domain provided herein, or the C-terminal 90%, 85%, 80%, 75%, 70%, 65%, 60%, 55%, 50%, 45%, 40%, 35%, 30%, 25%, 20%, 15%, 10%, or 5% or more of a Cas protein domain (e.g., as set forth Table 8). The N-terminal portion may correspond to the N-terminal 95% or more of the amino acids of a Cas protein domain provided herein, or the N-terminal 90%, 85%, 80%, 75%, 70%, 65%, 60%, 55%, 50%, 45%, 40%, 35%, 30%, 25%, 20%, 15%, 10%, or 5% or more of a Cas domain provided herein (e.g., as set forth Table 8).

In some embodiments, the circular permutant can be formed by linking a C-terminal fragment of a Cas protein domain provided herein to an N-terminal fragment of a Cas protein domain provided herein, either directly or by using a linker, such as an amino acid linker. In some embodiments, the C-terminal fragment that is rearranged to the N-terminus includes or corresponds to the C-terminal 30% or less of the amino acids of a Cas protein domain provided herein (e.g., as set forth Table 8). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas protein domain provided herein (e.g., as set forth Table 8). In some embodiments, the C-terminal fragment that is rearranged to the N-terminus, includes or corresponds to the C-terminal 410 residues or less of a Cas protein domain provided herein (e.g., as set forth Table 8). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas protein domain provided herein (e.g., as set forth Table 8). In some embodiments, the C-terminal portion that is rearranged to the N-terminus includes or corresponds to the C-terminal 357, 341, 328, 120, or 69 residues of a Cas protein domain provided herein (e.g., as set forth Table 8).

Prime editors described herein may also comprise Cas proteins other than Cas9. For example, a second polypeptide of a prime editor as described herein may comprise a Cas12a (Cpf1) polypeptide or variants thereof. In some embodiments, the Cas12a polypeptide may comprise a mutation that reduces or abolishes the endonuclease domain of the Cas12a polypeptide. For example, the Cas12a polypeptide may be a Cas12a nickase. In some instances, the Cas protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally occurring Cas12a polypeptide.

In some embodiments, the DNA binding domain of a prime editor may be a Cas protein that comprises a Cas12b (C2c1) or a Cas12c (C2c3) polypeptide. In some instances, the Cas protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12b (C2c1) or Cas12c (C2c3) protein. In some instances, the Cas protein is a Cas12b nickase or a Cas12c nickase. In some instances, the Cas protein is a Cas12e, a Cas12d, a Cas13, or a Cas (1) polypeptide. In some instances, the Cas protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e, Cas12d, Cas13, or Cas Φprotein. In some instances, the Cas protein is a Cas12e, Cas12d, Cas13, or Cas Φnickase.

### Prime Editor Linker

In some embodiments, polypeptide domains of a prime editor may be fused or linked by a linker, e.g., a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a DNA polymerase domain (e.g., a reverse transcriptase domain) associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

Linkers

Polypeptides comprising components of a prime editor may be fused via linkers, e.g., peptide linkers. In some embodiments, the linker region can be truncated and/or modified. In some embodiments, a prime editor may comprise a linker as described in Table 11. In some embodiments, a linker may comprise a sequence that is at least about 50%, 60%, 70%, 80%, 90%, 95%, 99%, identical to a sequence set forth in SEQ ID NOs: 273-318. In some embodiments, a linker may comprise a sequence set forth in any ne of SEQ ID NOs: 272-318. In some embodiments, polypeptides comprising components of a prime editor may be provided in trans relevant to each other. For example, a reverse transcriptase domain provided herein may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain. In some embodiments, a DNA binding domain of a prime editor may be located C-terminal to the DNA polymerase domain. In some embodiments, a DNA binding domain of a prime editor may be located N-terminal to the DNA polymerase domain

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 911). In some embodiments, the amino acid sequence of the MCP is:

(SEQ ID NO: 912)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIAANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain aspects, the prime editors provided herein comprise a linker between the Cas protein domain and the prime editing domain (e.g., reverse transcriptase domain). The linker may affect the editing efficiency by the prime editor as well as the stability and half-life of the prime editor.

As described herein, the linker may be between 10 and 200 amino acids in length, between 15 and 155 amino acids in length, or at least 30 and 300 amino acids in length. The linker may be at least 10 amino acids in length, at least 20 amino acids in length, at least 30 amino acids in length, at least 40 amino acids in length, or at least 50 amino acids in length. In some embodiments, the linker is about 15 amino acids in length. In some embodiments, the linker is about 25 amino acids in length. In some embodiments, the linker is about 50 amino acids in length. In some embodiments, the linker is about 100 amino acids in length. In some embodiments, the linker is about 155 amino acids in length.

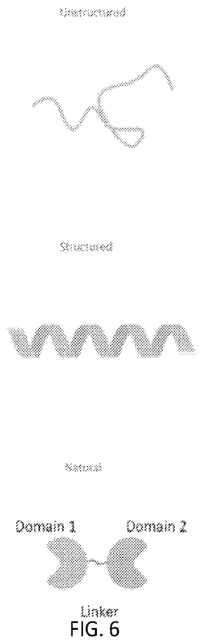

It should be appreciated that the linker of this disclosure may be an unstructured linker, a structured linker, or a natural linker. As used herein, a “natural linker” is a linker that has evolved by nature to maintain activity between protein domains (e.g., between Cas protein domain and reverse transcriptase domain). In some embodiments, the natural linker comprises at least 1 domain, at least 2 domains, at least 3 domains, at least 4 domains, at least 5 domains, at least 6 domains, at least 7 domains, at least 8 domains, at least 9 domains, or at least 10 domains. An exemplary natural linker is shown in FIG. 6.

As use herein, a “structured linker” is a linker that maintains structural integrity and increases stability of protein domains (e.g., between Cas protein domain and reverse transcriptase domain). In some embodiments, the structured linker comprises a fixed distance between protein domains (e.g., 1 domain, 2 domains, 3 domains, 4 domains, 5 domains, 6 domains, 7 domains, 8 domains, 9 domains, or 10 domains). In some embodiments, the fixed distance is at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, or at least 10 nucleotides. An exemplary structured linker is shown in FIG. 6.

As used herein, an “unstructured linker” is a linker that is structurally flexible and allows access to productive conformations of protein domains (e.g., between Cas protein domain and reverse transcriptase domain). In some embodiments, the unstructured linker comprises at least 1 domain, at least 2 domains, at least 3 domains, at least 4 domains, at least 5 domains, at least 6 domains, at least 7 domains, at least 8 domains, at least 9 domains, or at least 10 domains. An exemplary unstructured linker is shown in FIG. 6.

The linker of the disclosure may comprise a PE2 linker, a GS11 linker, a ALEA linker, a GSS linker, a NAT17 linker, a NAT 23 linker, or a GS8 linker.

In some embodiments, the linker is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence provided in Table 11. In some embodiments, the linker comprises an amino acid sequence selected from SEQ ID NO: 272-318 (Table 11). Exemplary linkers are shown in Table 11.

In some embodiments, a prime editing complex comprising a prime editor linker disclosed herein has at least 0.5, at least 1, at least 1.1, at least 1.2, at least 1.25, at least 1.3, at least 1.4, at least 1.5, at least 1.6, at least 1.7 at least 1.8, at least 1.9, or at least 2.0 fold average increase in editing efficiency over a PE2 system.

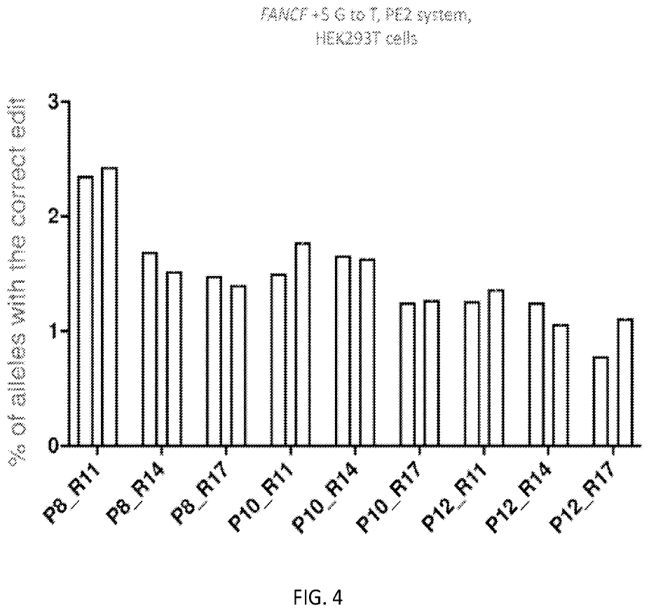

A person of skill in the art would appreciate that the present disclosure is not limited by the sequences in Tables 11 and structures in FIG. 6 as the configurations in Tables 11 and FIG. 4 are examples of a broader class of linkers included in the present disclosure.

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

A linker may be flexible, rigid, and/or cleavable. In some embodiments, the linker is a flexible linker. In some embodiments, the linker comprises the amino acid sequence (GGGGS)n (SEQ ID NO: 913), (G)n (SEQ ID NO: 914), (EAAAK)n (SEQ ID NO: 915), (GGS)n (SEQ ID NO: 916), (SGGS)n (SEQ ID NO: 917), (XP)n (SEQ ID NO: 918), or any combination thereof, wherein n is independently an integer between 1 and 30, and wherein X is any amino acid. In some embodiments, the linker comprises the amino acid sequence (GGS)n, wherein n is 1, 3, or 7(SEQ ID NO: 919). In some embodiments, the linker comprises the amino acid sequence SGSETPGTSESATPES (SEQ ID NO: 920). In some embodiments, the linker comprises the amino acid sequence SGGSSGGSSGS ETPGTSESATPESSGGSSGGS (SEQ ID NO: 921). In some embodiments, the linker comprises the amino acid sequence SGGSGGSGGS (SEQ ID NO: 922). In some embodiments, the linker comprises the amino acid sequence SGGS (SEQ ID NO: 923). In other embodiments, the linker comprises the amino acid sequence

(SEQ ID NO: 924)
SGGSSGGSSGSETPGTSESATPESAGSYPYDVPDYAGSAAPAAKKKKLD
GSGSGGSSGGS.

In some embodiments, a linker comprises 1-100 amino acids. In some embodiments, the linker comprises the amino acid sequence SGSETPGTSESATPES (SEQ ID NO: 920). In some embodiments, the linker comprises the amino acid sequence SGGSSGGSSGSETPGTSESATPESSGGSSGGS (SEQ ID NO: 921). In some embodiments, the linker comprises the amino acid sequence SGGSGGSGGS (SEQ ID NO: 922). In some embodiments, the linker comprises the amino acid sequence SGGS (SEQ ID NO: 923). In some embodiments, the linker comprises the amino acid sequence GGSGGS (SEQ ID NO: 925), GGSGGSGGS (SEQ ID NO: 926), or SGGSSGGSSGSETPGTSESATPESAGSYPYDVPDYAGSAAPAAKKKKLDGSGSGGSSGGS (SEQ ID NO: 924), or SGGSSGGSSGSETPGTSESATPESSGGSSGGSS (SEQ ID NO: 316).

In some embodiments the amino acid linkers are homologous to the endogenous amino acids that exist between such domains in a native polypeptide. In some embodiments the endogenous amino acids that exist between such domains are substituted but the length is unchanged from the natural length.

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]—[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell.

In some embodiments, a prime editor fusion protein is PE1. In some embodiments, a prime editor fusion protein comprises one or more individual components of PE1, as disclosed in WO/2020/191234, which is hereby incorporated by reference. In some embodiments, a PE1 prime editor fusion protein comprises a RT domain having an amino acid sequence as set forth in SEQ ID NO: 855. In some embodiments, a PE1 prime editor fusion protein comprises a RT domain having an amino acid sequence as set forth in SEQ ID NO: 857. In some embodiments, a prime editor fusion protein is PE2, as disclosed in WO/2020/191234. In some embodiments, a prime editor fusion protein comprises one or more individual components of PE2. The amino acid sequence of an exemplary PE2 and its individual components in shown in Table 15 and 16.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the prime editor fusion sequences described herein or known in the art.

Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In some embodiments, a NLS may be linked or fused to the C-terminus of a DNA binding domain. In some embodiments, a NLS may be linked or fused to the N-terminus of a DNA binding domain. In some embodiments, a NLS may be linked or fused to the C-terminus of a DNA polymerase domain. In some embodiments, a NLS may be linked or fused to the N-terminus of a DNA polymerase domain. In some embodiments, a first NLS may be linked or fused to the C-terminus of a DNA binding domain and a second NLS may be linked or fused to the N-terminus of a DNA binding domain. In some embodiments, a NLS may be linked or fused to the N-terminus of a DNA polymerase domain. In some embodiments, a first NLS may be linked or fused to the C-terminus of a DNA polymerase domain and a second NLS may be linked or fused to the N-terminus of a DNA polymerase domain. In some embodiments, a first NLS may be linked or fused to the C-terminus of a DNA binding domain and a second NLS may be linked or fused to the N-terminus of a DNA polymerase domain. In some embodiments, a first NLS may be linked or fused to the C-terminus of a DNA polymerase domain and a second NLS may be linked or fused to the N-terminus of a DNA binding domain. In some embodiments, the first and the second NLs are identical. In some embodiments, the first and the second NLS are different.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor may further comprise more than 4, 5, 6, 7, 8, 9 or 10 NLSs.

In addition, the NLSs may be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a prime editor comprises an amino acid sequence comprising a nuclear localization signal (NLS) having the sequence

(SEQ ID NO: 524)
MDSLLMNRRKFLYQFKNVRWAKGRRETYLC,
(SEQ ID NO: 927)
KRTADGSEFESPKKKRKV,
(SEQ ID NO: 928)
KRTADGSEFEPKKKRKV,
(SEQ ID NO: 929)
NLS KRPAAIKKAGQAKKKK,
(SEQ ID NO: 930)
RQRRNELKRSF,
or
(SEQ ID NO: 931)
NQSSNFGPMKGGNFGGRSSGPYGGGGQYFAKPRNQGGY.

In some embodiments, a NLS is a monopartite NLS. For example, in some embodiments, a NLS is a SV40 large T antigen NLS PKKKRKV(SEQ ID NO: 522). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the Xenopus nucleoplasmin sequence KRXXXXXXXXXXKKKL (SEQ ID NO: 922) wherein X is any amino acid. In some embodiments, a NLS is a noncanonical sequences such as M9 of the hnRNP A1 protein, the influenza virus nucleoprotein NLS, and the yeast Gal4 protein NLS.

Other non-limiting examples of NLS sequences are provided in Table 12 below.

### Engineered Cas-RT

In some embodiments. prime editors described herein comprise an engineered Cas-RT fusion protein or protein complexes, or a DNA binding domain (e.g., a Cas protein) -DNA polymerase (e.g., RT) fusion proteins or complexes. For example, a prime editor may comprise a fusion protein of a Cas polypeptide and a RT polypeptide, where the Cas component of a naturally occurring fusion protein of a Cas-RT and a Type III Cas protein is replaced by a heterologous DNA binding domain, e.g., a heterologous Cas polypeptide. In some embodiments, a prime editor comprises a RT domain of a naturally occurring Cas-RT fusion protein, and a DNA binding domain that is heterologous Cas polypeptide, i.e., the Cas polypeptide that is different that the one in the corresponding naturally occurring Cas-RT fusion protein. In some embodiments, a prime editor comprises a DNA binding domain and a RT domain that naturally occurs in a Type III CRISPR system Cas-RT fusion protein. In some embodiments, the Cas polypeptide in a naturally occurring Cas-RT fusion protein that is replaced by a heterologous Cas polypeptide comprises a Cas1 polypeptide, a Cas3 polypeptide, or a Cas6 polypeptide. In some embodiments, a prime editor comprises a DNA binding domain and an RT domain that naturally occurs in a Cas1-RT fusion protein (the RT also referred to as a “Cas1 RT”), and the DNA binding domain is not a Cas1 domain. In some embodiments, a prime editor comprises a DNA binding domain comprises a DNA binding domain and an RT domain that naturally occurs in a Cas6-RT fusion protein (the RT also referred to as a “Cas6 RT”), and the DNA binding domain is not a Cas6 domain. In some embodiments, a prime editor comprises a DNA binding domain comprises a DNA binding domain and an RT domain that naturally occurs in a Cas-RT fusion protein comprising Cas1 and Cas6 ((the RT also referred to as a “Cas1-Cas6 RT”), and the DNA binding domain is not a Cas1 or Cas6 domain. The naturally occurring Cas-RT fusion may have a configuration of Cas1-RT-Cas6 or Cas6-RT-Cas1, where either of the Cas1 or Cas6 may be replaced by a heterologous DNA binding domain for the purpose of a prime editor. In some embodiments, the DNA binding domain-RT fusion or complex may further be able to interact with other CRISPR Cas proteins, e.g., Cas3. As used herein, “heterologous” means a non-native gene or protein component of, e.g., an engineered complex or fusion protein that does not naturally occur in the same organism, or in a naturally occurring fusion protein or complex, as other components of the complex or fusion protein, but which is engineered into the complex or fusion protein.

The DNA binding domain that is used to replace a Cas domain in a naturally occurring Cas-RT fusion can be any DNA binding domain as described herein. In some embodiments, the DNA binding domain comprises a Cas9 polypeptide, or any functional variant or fragment as described herein. In some embodiments, the DNA binding domain comprises a Cas12a polypeptide, or any functional variant or fragment as described herein. In some embodiments, the DNA binding domain comprises a Cas12b, Cas12c, Cas12d, Cas 12e, or a Cas (1) polypeptide or any functional variant or fragment as described herein. In some embodiments, the DNA binding domain comprises nuclease activity, for example, a nickase activity.

In some embodiments, the DNA binding domain comprises a Cas9 polypeptide or a Cas9 variant as described herein, or a functional fragment thereof. In some embodiments, the DNA binding domain comprises a Cas9 nickase as disclosed herein, e.g., a H840A Cas9 nickase.

In some embodiments, a RT domain of a Cas-RT comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID Nos: 129-136, 345, 368, 396, or 533-846. In some embodiments, a RT domain of a Cas-RT comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions or substitutions compared to any of the amino acid sequences set forth in SEQ ID NO: 129-136 or 533-846. In some embodiments, the RT domain of a Cas-RT comprises an amino acid sequence selected from the group consisting of: SEQ ID Nos: 129-136, 345, 368, 396, or 533-846.

In some embodiments, a prime editor comprising a Cas9 polypeptide and an RT domain (e.g., RT domain of a naturally occurring Cas-RT) that comprises an amino acid sequence that is at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a sequence selected from the group consisting of: SEQ ID Nos: 129-136, 345, 368, 396, or 533-846. In some embodiments, a prime editor comprising a Cas9 polypeptide and an RT domain (e.g., RT domain of a naturally occurring Cas-RT) comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions or substitutions compared to any of the amino acid sequences set forth in SEQ ID NO: 129-136, 345, 368, 396, or 533-846. In some embodiments, a prime editor comprising a Cas9 polypeptide and an RT domain (e.g., RT domain of a Cas-RT) comprises an amino acid sequence selected from the group consisting of: SEQ ID Nos: 129-136, 345, 368, 396, or 533-846.

The engineered Cas-RTs described herein may comprise additional functional domains, for example, any of the SET domains as described herein. In some embodiments, an engineered Cas-RT comprises a GB1 domain, or bGB1 domain described herein. In some embodiments, the engineered Cas-RT described herein and fragments thereof may also comprise any of the SET domain, GB1 domain, or bGB1 domain described herein. The SET domain, GB1 domain, or bGB1 domain may comprise any SET domains, GB1 domains, or bGB1 domains described in this disclosure.

### Domain Structures of Prime Editors

In some embodiments, a prime editor may comprise a fusion polypeptide comprising a first polypeptide and a second polypeptide. In some embodiments, a first polypeptide of a prime editor may be located at the N-terminus or C-terminus of the second polypeptide of the primer editor. In some embodiments, the first polypeptide is located at the N-terminus of the second polypeptide. In some embodiments, the first polypeptide is located at the C-terminus of the second polypeptide.

In some embodiments, a prime editor may further comprise at least one nuclear localization sequence (NLS). In some embodiments, a prime editor may further comprise 1 NLS. In some embodiments, a prime editor may further comprise 2 NLSs. In other cases, a prime editor may further comprise 3 NLSs. In one case, a primer editor may further comprise more than 4, 5, 6, 7, 8, 9 or 10 NLSs.

In some embodiments, the domain structure of a prime editor may comprise NH2-first polypeptide-second polypeptide-COOH or NH2-second polypeptide-first polypeptide-COOH, wherein NH2 is the N-terminus of the primer editor, wherein COOH is the C-terminus of the prime editor, wherein—comprises from 0-100 amino acids. In some embodiments, the domain structure of a prime editor may comprise NH2-first polypeptide-second polypeptide-COOH. In some embodiments, the domain structure of a prime editor may comprise NH2-second polypeptide-first polypeptide-COOH.

In some instance, the domain structure of a prime editor comprising one NLS may comprise NH2-first polypeptide-second polypeptide-NLS-COOH, NH2-first polypeptide-NLS-second polypeptide-COOH, NH2-NLS-first polypeptide-second polypeptide-COOH, NH2-second polypeptide-first polypeptide-NLS—COOH, NH2-second polypeptide-NLS-first polypeptide-COOH, or NH2-NLS-second polypeptide-first polypeptide-COOH. In some embodiments, the domain structure of a prime editor comprising one NLS may comprise NH2-first polypeptide-second polypeptide-NLS—COOH. In some embodiments, the domain structure of a prime editor comprising one NLS may comprise NH2-first polypeptide-NLS-second polypeptide-COOH. In some embodiments, the domain structure of a prime editor comprising one NLS may comprise NH2-NLS-first polypeptide-second polypeptide-COOH. In some embodiments, the domain structure of a prime editor comprising one NLS may comprise NH2-second polypeptide-first polypeptide-NLS—COOH. In some embodiments, the domain structure of a prime editor comprising one NLS may comprise NH2-second polypeptide-NLS-first polypeptide-COOH. In some embodiments, the domain structure of a prime editor comprising one NLS may comprise NH2-NLS-second polypeptide-first polypeptide-COOH. In some embodiments, a prime editor comprising more than one NLS may have an NLS sequence located in the N-terminus or C-terminus of the first polypeptide or the second polypeptide. In some embodiments, an NLS sequence may be located in the N-terminus or C-terminus of another NLS.

In some embodiments, a second polypeptide may comprise a DNA-binding domain or a DNA endonuclease domain. In some embodiments, a second polypeptide may comprise a DNA-binding domain. In some embodiments, the second polypeptide of a prime editor may comprise a DNA endonuclease domain. In one case, a second polypeptide may comprise a DNA-binding domain and a DNA endonuclease domain. In some embodiments, the DNA-binding domain may comprise DNA endonuclease activity. In some embodiments, the DNA-binding domain and the DNA endonuclease domain may comprise overlapping amino acids. In some embodiments, the prime editor may comprise a DNA-binding domain and further a separate endonuclease domain. For example, the DNA-binding domain and the DNA endonuclease domain may comprise two independent amino acid sequences. In other cases, the second polypeptide of a prime editor may comprise more than one DNA-binding domain. In one case, the second polypeptide of a prime editor may comprise more than one DNA endonuclease domain.

In some embodiments, the DNA-binding domain of a prime editor may be located at the N-terminus or C-terminus of the DNA endonuclease domain of the prime editor. In some embodiments, the DNA-binding domain is located at the N-terminus of the DNA endonuclease domain of the prime editor. In some embodiments, the DNA-binding domain of a prime editor is located at the C-terminus of the DNA endonuclease domain of the prime editor. In some embodiments, the DNA-binding domain of a prime editor is located within the DNA endonuclease domain of the prime editor. In other cases, the DNA endonuclease domain of a prime editor is located within the DNA-binding domain of the prime editor. For the prime editor comprising more than one DNA-binding domain, the DNA-binding domain may also be located at the N-terminus and the C-terminus of a DNA endonuclease domain. For the prime editor comprising more than one DNA endonuclease domain, the DNA endonuclease domain may also be located at the N-terminus and the C-terminus of a DNA-binding domain. In some embodiments, a prime editor may comprise the combinations of the arrangements of the DNA-binding domain and the DNA endonuclease domain described in this disclosure.

Flap Endonuclease

In some embodiments, a prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a- trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C, essentially in same way as a contiguous intein does. In some embodiments, a split-intein is derived from a eukaryotic intein, a bacterial intein, or an archaeal intein. Preferably, the split intein so derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a prime editor comprises a protein domain that is capable of modifying the intracellular half-life of the prime editor.

Components of a prime editors may be arranged in a modular fashion to target, edit, or modify a target DNA sequence, e.g., to install a desired nucleotide edit into a target cell genome, by reverse transcription. In some embodiments, the components of a prime editor may comprise an unrelated DNA binding domain, and a DNA polymerase domain, e.g., reverse transcriptase domain. In some embodiments, the DNA binding domain and the DNA polymerase domain can be interchangeably located in the 5′ portion of the prime editor or the 3′ portion of the prime editor. In some embodiments, multiple functional domains may arise from a single protein. In some embodiments, all functional domains may arise from different proteins. In some embodiments, a DNA binding domain of a prime editor may be located C-terminal to the DNA polymerase domain. In some embodiments, a DNA binding domain of a prime editor may be located N-terminal to the DNA polymerase domain.

In some embodiments, a prime editor may comprise a DNA polymerase domain having an amino acid sequence that is selected from any of the amino acid sequence selected from SEQ ID NOs: 1-95, 138-146, 198-271, 319-493, 855-857, 884, or 990-1006, a DNA binding domain having an amino acid sequence that is selected from any of the amino acid sequence selected from SEQ ID NOs: 138-146, 494, 858, 1100, 495-503, and optionally a linker having an amino acid sequence that is selected from any of the amino acid sequence selected from SEQ ID NOs: 272-318. In some embodiments, a prime editor further comprises one or more nuclear localization sequence (NLS) having an amino acid sequence that is selected from any of the amino acid sequence selected from SEQ ID NOs: 522-532 or described herein. In some embodiments, the NLS is fused to the N-terminus of a DNA polymerase domain described herein. In some embodiments, the NLS is fused to the C-terminus of the DNA polymerase domain. In some embodiments, the NLS is fused to the N-terminus or the C-terminus of a DNA binding domain. In some embodiments, a linker sequence is disposed between the NLS and a domain of the prime editor.

PE gRNAs

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene or may refer to the edit encoded by the editing template on the newly synthesized single stranded DNA that replaces the editing target sequence, as compared to the editing target sequence.

In some embodiments, a PEgRNA comprises at least one of: a spacer, an extension arm, and a gRNA core. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence can be referred to as an extension arm.

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase iii domain. The reverse transcriptase editing template may also be referred to herein as an RT template, or RTT. In some embodiments, the editing template comprises partial complementarity to an editing target sequence in the target gene. In some embodiments, the editing template comprises substantial or partial complementarity to the editing target sequence except at the position of the intended nucleotide edits to be incorporated into the target gene.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

In some embodiments, a spacer may comprise a sequence that hybridizes to a first strand of a double stranded target DNA sequence. For example, the spacer may comprise complementary sequence to a protospacer sequence in the first strand of the double stranded DNA sequence. In some cases, an extension arm may comprise a sequence that hybridizes to a second strand (i.e., the complementary strand of the first strand) of the double stranded target DNA sequence. In some embodiments, a gRNA core may comprise a sequence that interacts with the second polypeptide. In some instances, a nucleotide of a PEgRNA may be part of a spacer. In some cases, a nucleotide of a PEgRNA may be part of an extension arm. In some cases, a nucleotide of a PEgRNA may be part of a gRNA core. In some embodiments, a nucleotide of a PEgRNA may be part of a spacer and an extension arm. In some embodiments, a nucleotide of a PEgRNA may be part of a spacer and a gRNA core. In some embodiments, a nucleotide of a PEgRNA may be part of an extension arm and a gRNA core. In some embodiments, a nucleotide of a PEgRNA may be part of a spacer and an extension arm. In some embodiments, a nucleotide of a PEgRNA may not be part of a spacer, an extension arm, or a gRNA core.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing target, a PBS, a spacer, and a gRNA core.

Provided herein in some embodiments are example sequences for PEgRNAs, including PEgRNA spacers, PBS, RTT, and ngRNA spacers for a prime editing system comprising a nuclease that recognizes the PAM sequence “NG.” In some embodiments, a PAM motif on the edit strand comprises an “NG” motif, wherein N is any nucleotide. In some embodiments, a PEgRNA of this disclosure is part of a prime editing system that recognizes the PAM motif GG and comprises a spacer in, a PBS sequence, and an RTT sequence. In some embodiments, a PEgRNA of this disclosure is part of a prime editing system that recognizes the PAM motif TG. In some embodiments, a PEgRNA of this disclosure is part of a prime editing system that recognizes the PAM motif CG. In some embodiments, a PEgRNA of this disclosure is part of a prime editing system that recognizes the PAM motif AG.

Provided herein in some embodiments are example sequences for PEgRNA spacers, PBS, RTT, and ngRNA spacers for a prime editing system comprising a nuclease that recognizes the PAM sequence “NAG.” In some embodiments, a PAM motif on the edit strand comprises an “NAG” motif, wherein N is any nucleotide.

In some embodiments, a PEgRNA of this disclosure is part of a prime editing system that recognizes the PAM motif GAG. In some embodiments, a PEgRNA of this disclosure is part of a prime editing system that recognizes the PAM motif CAG.

Provided herein in some embodiments are example sequences for PEgRNA spacers, PBS, RTT, and ngRNA spacers for a prime editing system comprising a nuclease that recognizes the PAM sequence “NGA.” In some embodiments, a PAM motif on the edit strand comprises an “NGA” motif, wherein N is any nucleotide. In some embodiments, a PEgRNA of this disclosure is part of a prime editing system that recognizes the PAM motif GGA. In some embodiments, a PEgRNA of this disclosure is part of a prime editing system that recognizes the PAM motif TGA.

Provided herein in some embodiments are example sequences for PEgRNA spacers, PBS, RTT, and ngRNA spacers for a prime editing system comprising a nuclease that recognizes the PAM sequence “NGG.” In some embodiments, a PAM motif on the edit strand comprises an “NGG” motif, wherein N is any nucleotide. In some embodiments, a PEgRNA of this disclosure is part of a prime editing system that recognizes the PAM motif CGG. In some embodiments, a PEgRNA of this disclosure is part of a prime editing system that recognizes the PAM motif AGG.

Provided herein in some embodiments are example sequences for PEgRNA spacers, PBS, RTT, and ngRNA spacers for a prime editor complex comprising a nuclease that recognizes the PAM sequence “NNGG.” In some embodiments, a PAM motif on the edit strand comprises an “NNGG” motif, wherein N is any nucleotide. In some embodiments, a PEgRNA of this disclosure is part of a prime editing system that recognizes the PAM motif GCGG. In some embodiments, a PEgRNA of this disclosure is part of a prime editing system that recognizes the PAM motif GAGG.

Provided herein in some embodiments are example sequences for PEgRNA spacers, PBS, RTT, and ngRNA spacers for a prime editing system comprising a nuclease that recognizes the PAM sequence “NNGRRT.” In some embodiments, a PAM motif on the edit strand comprises an “NNGRRT” motif, wherein N is any nucleotide and R is A or G. In some embodiments, a PEgRNA of this disclosure is part of a prime editing system that recognizes the PAM motif AAGGGT. In some embodiments, the PEgRNA recognizes the PAM motif GGGGAT.

Provided herein in some embodiments are example sequences for PEgRNA spacers, PBS, RTT, and ngRNA spacers for a prime editor complex comprising a nuclease that recognizes the PAM sequence “NGA.” In some embodiments, a PAM motif on the edit strand comprises an “NGA” motif, wherein N is any nucleotide.

Provided herein in some embodiments are example sequences for PEgRNA spacers, PBS, RTT, and ngRNA spacers for a prime editor complex comprising a nuclease that recognizes the PAM sequence “NGN.” In some embodiments, a PAM motif on the edit strand comprises an “NGN” motif, wherein N is any nucleotide.

Provided herein in some embodiments are example sequences for PEgRNA spacers, PBS, RTT, and ngRNA spacers for a prime editor complex comprising a nuclease that recognizes the PAM sequence “NGA.” In some embodiments, a PAM motif on the edit strand comprises an “NRN” motif, wherein N is any nucleotide and R is A or G.

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA can comprise a first polynucleotide comprising the spacer and a first portion of a gRNA core comprising, which can be also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem.

In some embodiments, a spacer sequence comprises a region that has substantial complementarity to a search target sequence on the target strand of a double stranded target DNA. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence. In some embodiments, a spacer may guide a prime editing complex to a genomic locus with identical sequence during prime editing.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

In some embodiments, the spacer comprises a first spacer sequence comprising the 5′ half of the spacer and a second spacer sequence comprising the 3′ half of the spacer, wherein the tag sequence is between the first spacer sequence and the second spacer sequence. In some embodiments, the tag sequence does not have substantial complementarity to the spacer. In some embodiments, the tag does not have complementarity to the spacer.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

The extension arm of a PEgRNA can comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA can comprise a primer binding site sequence (PBS, or PBS sequence) that hybridizes with a free 3′ end of a single stranded DNA in the target generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS can be complementary or substantially complementary to a DNA sequence in the edit strand of the target gene. By annealing with the edit strand at a free hydroxy group, e.g., a free 3′ end generated by prime editor nicking, the PBS can initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene. In some embodiments, the PBS is perfectly complementary, or 100% complementary, to a region of the edit strand of the target gene.

An extension arm of a PEgRNA may comprise an editing template that serves as a DNA synthesis template for the DNA polymerase in a prime editor during prime editing.

The length of an editing template can vary depending on, e.g., the prime editor components, the search target sequence, and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated into the target gene. In some embodiments, the editing template comprises a nucleotide sequence comprising about 85% to about 95% complementarity to an editing target sequence in the edit strand in the target gene. In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene.

Nucleotide Editing

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA can comprise one or more intended nucleotide edits, compared to the target gene to be edited. Position of the intended nucleotide edit(s) relevant to other components of the PEgRNA, or to particular nucleotides (e.g., mutations) in the target gene can vary. In some embodiments, the nucleotide edit is in a region of the PEgRNA corresponding to or homologous to the protospacer sequence. In some embodiments, the nucleotide edit is in a region of the PEgRNA corresponding to a region of the target gene outside of the protospacer sequence.

In some embodiments, the position of a nucleotide edit incorporation in the target gene can be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream of the 5′ most nucleotide of the PAM sequence in the edit strand of the target gene. By 0 base pair upstream or downstream of a reference position, it is meant that the intended nucleotide is immediately upstream or downstream of the reference position. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream of the 5′ most nucleotide of the PAM sequence. In some embodiments, the nucleotide edit is incorporated at a position corresponding to 3 base pairs upstream of the 5′ most nucleotide of the PAM sequence. In some embodiments, the nucleotide edit in is incorporated at a position corresponding to 4 base pairs upstream of the 5′ most nucleotide of the PAM sequence. In some embodiments, the nucleotide edit is incorporated at a position corresponding to 5 base pairs upstream of the 5′ most nucleotide of the PAM sequence. In some embodiments, the nucleotide edit in the editing template is at a position corresponding to 6 base pairs upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs downstream of the 5′ most nucleotide of the PAM sequence in the edit strand of the target gene. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs downstream of the 5′ most nucleotide of the PAM sequence. In some embodiments, a nucleotide edit is incorporated at a position corresponding to 3 base pairs downstream of the 5′ most nucleotide of the PAM sequence. In some embodiments, a nucleotide edit is incorporated at a position corresponding to 4 base pairs downstream of the 5′ most nucleotide of the PAM sequence. In some embodiments, a nucleotide edit is incorporated at a position corresponding to 5 base pairs downstream of the 5′ most nucleotide of the PAM sequence. In some embodiments, a nucleotide edit is incorporated at a position corresponding to 6 base pairs downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene can be determined based on position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template can be referred to by aligning the editing template with the partially complementary editing target sequence on the edit strand and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand can be referred to as at positions +1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity can also be referred to as position 0 in the editing template, the nucleotides in the editing template corresponding to the nucleotides at positions +1, +2, +3, +4, . . . , +n on the PAM strand of the double stranded target DNA can also be referred to as at positions +1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions +1, +2, +3, +4, . . . , +n are 5′ of position 0 and positions −1, −2, −3, −4, . . . −n are 3′ of position 0 in the editing template. In some embodiments, an intended nucleotide edit is at position+n of the editing template relative to position 0. Accordingly, the intended nucleotide edit may be incorporated at position+n of the PAM strand of the double stranded target DNA (and subsequently, the target strand of the double stranded target DNA) by prime editing. The number n may be referred to as the nick to edit distance.

When referred to in the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

The corresponding positions of the intended nucleotide edit incorporated in the target gene may also be referred to be based on the nicking position generated by a prime editor based on sequence homology and complementarity. For example, in embodiments, the distance between the nucleotide edits to be incorporated into the target gene and the nick generated by the prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template extends beyond a nucleotide edit to be incorporated to the target gene sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 base pairs 3′ to the nucleotide edit to be incorporated to the target gene sequence. In some embodiments, the editing template comprises at least 4 to 30 base pairs 3′ to the nucleotide edit to be incorporated to the target gene sequence. In some embodiments, the editing template comprises at least 4 to 25 base pairs 3′ to the nucleotide edit to be incorporated to the target gene sequence. In some embodiments, the editing template comprises at least 4 to 20 base pairs 3′ to the nucleotide edit to be incorporated to the target gene sequence. In some embodiments, the editing template comprises at least 4 to 30 base pairs 5′ to the nucleotide edit to be incorporated to the target gene sequence. In some embodiments, the editing template comprises at least 4 to 25 base pairs 5′ to the nucleotide edit to be incorporated to the target gene sequence. In some embodiments, the editing template comprises at least 4 to 20 base pairs 5′ to the nucleotide edit to be incorporated to the target gene sequence.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5 ‘-spacer-gRNA core-RTT-PBS-3’ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g. by reverse transcription) to replace a target sequence in the target gene. In some embodiments, the editing target sequence in the edit strand of the target gene is replaced by the newly synthesized strand, and the nucleotide edit(s) are incorporated in the region of the target gene. In some embodiments, the target gene is a target gene. In some embodiments, the editing template of the PEgRNA encodes a newly synthesized single stranded DNA that comprises a wild type target gene sequence. In some embodiments, the newly synthesized DNA strand replaces the editing target sequence in the target gene, wherein the editing target sequence (or the endogenous sequence complementary to the editing target sequence on the target strand of the target gene) comprises a mutation compared to a wild type target gene.

In some embodiments, the editing target sequence comprises a mutation in an intron of the target gene as compared to a wild type target gene. In some embodiments, the editing target sequence comprises a mutation in an intron of the target gene that results in altered or aberrant splicing of a transcript encoded by the target gene compared to a transcript encoded by a wild type target gene.

In some embodiments, the editing template comprises one or more intended nucleotide edits compared to the sequence on the target strand of the target gene that is complementary to the editing target sequence. In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits.

In some embodiments, incorporation of the one or more intended nucleotide edits corrects the mutation in the editing target sequence to wild type nucleotides at corresponding positions in the target gene. As used herein, “correcting” a mutation means restoring a wild type sequence at the place of the mutation in the double stranded target DNA, e.g., target gene, by prime editing. In some embodiments, the editing template comprises and/or encodes a wild type target gene sequence.

In some embodiments, incorporation of the one or more intended nucleotide edits does not correct the mutation in the editing target sequence to wild type sequence but allows for expression of a functional protein encoded by the target gene.

In some embodiments, the editing template comprises one or more intended nucleotide edits compared to the sequence on the target strand of the target gene that is complementary to the editing target sequence, wherein the one or more intended nucleotide edits is a single nucleotide substitution, polynucleotide substitution, nucleotide insertion, or nucleotide deletion. In some embodiments, the intended nucleotide edit in the editing template comprises a single nucleotide substitution, polynucleotide substitution, nucleotide insertion, or nucleotide deletion compared to the sequence on the target strand of the target gene that is complementary to the editing target at a position corresponding to a mutation in target gene, wherein the editing target sequence is on the sense strand of the target gene.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, the gRNA core comprises the sequence: GUUUUAGAGCUAGAAAUAGCAAGUUAAAAUAAGGCUAGUCCGUUAUCAACUUGAAA AAGUGGCACCGAGUCGGUGC (SEQ ID NO:933); GUUUGAGAGCUAGAAAUAGCAAGUUUAAAUAAGGCUAGUCCGUUAUCAACUUGAAA AAGUGGGACCGAGUCGGUCC (SEQ ID NO:896), or GUUUAAGAGCUAUGCUGGAAACAGCAUAGCAAGUUUAAAUAAGGCUAGUCCGUUAUC AACUUGAAAAAGUGGCACCGAGUCGGUGC (SEQ ID NO:899). In some embodiments, the gRNA core comprises the sequence

(SEQ ID NO: 933)
GUUUUAGAGCUAGAAAUAGCAAGUUAAAAUAAGGCUAGUCCGUUAUCAA
CUUGAAAAAGUGGCACCGAGUCGGUGC.

In some embodiments, the PEgRNA comprises a guide RNA (gRNA) core that associates with a DNA binding domain, e.g., a CRISPR-Cas protein domain, of a prime editor. In some embodiments, the gRNA core comprises a first gRNA core sequence comprising a 5′ half of the gRNA core and a second gRNA core sequence comprising a 3′ half of the gRNA core, and wherein the PEgRNA comprises, in 5′ to 3′ order: the spacer, the first gRNA core sequence, the editing template, the PBS, the tag sequence, and the second gRNA core sequence. The 5′ half and the 3′ half can form a functional gRNA core for association/binding with a programmable DNA binding protein, e.g., a Cas protein. One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor.

Any gRNA core sequences known in the art are also contemplated in the prime editing compositions described herein.

A PEgRNA can also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers can be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a PEgRNA or a nick guide RNA (ngRNA) can be chemically synthesized, or can be assembled or cloned and transcribed from a DNA sequence, e.g., a plasmid DNA sequence, or by any RNA oligonucleotide synthesis method known in the art. In some embodiments, DNA sequence that encodes a PEgRNA (or ngRNA) can be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) can be designed to append a nucleotide G at the 5′ end. Accordingly, in some embodiments, the PEgRNA (or nick guide RNA) can comprise an appended nucleotide G at the 5′ end. In some embodiments, a DNA sequence that encodes a PEgRNA (or nick guide RNA) can be designed to append a sequence that enhances transcription, e.g., a Kozak sequence, at the 5′ end. In some embodiments, a DNA sequence that encodes a PEgRNA (or nick guide RNA) can be designed to append the sequence CACC or CCACC at the 5′ end. Accordingly, in some embodiments, the PEgRNA (or nick guide RNA) can comprise an appended sequence CACC or CCACC at the 5′ end. In some embodiments, a DNA sequence that encodes a PEgRNA (or nick guide RNA) can be designed to append the sequence TTT, TTTT, TTTTT, TTTTTT, TTTTTTT at the 3′ end. Accordingly, in some embodiments, the PEgRNA (or nick guide RNA) can comprise an appended sequence UUU, UUUU, UUUUU, UUUUUU, or UUUUUUU at the 3′ end. In some embodiments, a PEgRNA or ngRNA may include a modifying sequence at the 3′ end having the sequence AACAUUGACGCGUCUCUACGUGGGGGCGCG (SEQ ID NO: 934).

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, the ngRNA is a guide RNA which contains a variable spacer sequence and a guide RNA scaffold or core region that interacts with the DNA binding domain, e.g., Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene. A prime editing system, composition, or complex comprising a ngRNA can be referred to as a “PE3” prime editing system, PE3 prime editing compositions, or PE3 prime editing complex.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system may be referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

In some embodiments, a PEgRNA (or ngRNA) comprises an additional secondary structure at the 5′ end. In some embodiments, a PEgRNA (or ngRNA) comprises an additional secondary structure at the 3′ end.

In some embodiments, the secondary structure comprises a pseudoknot. In some embodiments, the secondary structure comprises a pseudoknot derived from a virus. In some embodiments, the secondary structure comprises a pseudoknot of a Moloney murine leukemia virus (M-MLV) genome (a mpknot). In some embodiments, the secondary structure comprises a nucleotide sequence selected from the group consisting of GGGUCAGGAGCCCCCCCCCUGAACCCAGGAUAACCCUCAAAGUCGGGGGGCAACC (SEQ ID No: 935), GUCAGGGUCAGGAGCCCCCCCCCUGAACCCAGGAUAACCCUCAAAGUCGGGGGGCAA CCC (SEQ ID No: 859), GGGUCAGGAGCCCCCCCCCUGAACCCAGGAAAACCCUCAAAGUCGGGGGGCAACCC (SEQ ID No: 860), GGGUCAGGAGCCCCCCCCCUGCACCCAGGAAAACCCUCAAAGUCGGGGGGCAACCC (SEQ ID No: 861), GGGUCAGGAGCCCCCCCCCUGCACCCAGGAUAACCCUCAAAGUCGGGGGGCAACCC (SEQ ID No: 862), GUCAGGGUCAGGAGCCCCCCCCCUGAACCCAGGAAAACCCUCAAAGUCGGGGGGCAA CCC (SEQ ID No: 863), GUCAGGGUCAGGAGCCCCCCCCCUGCACCCAGGAAAACCCUCAAAGUCGGGGGGCAAC CC (SEQ ID No: 864), and GGGUCAGGAGCCCCCCCCCUGAACCCAGGAUAACCCUCAAAGUCGGGGGGC (SEQ ID No: 865), or a nucleotide sequence having at least 80%, 85%, 90%, 95%, 96%, 97%, 98%, or 99% sequence identity therewith. In some embodiments, the secondary structure comprises a nucleotide sequence of GGGUCAGGAGCCCCCCCCCUGAACCCAGGAUAACCCUCAAAGUCGGGGGGC (SEQ ID No: 865), or a nucleotide sequence having at least 80%, 85%, 90%, 95%, 96%, 97%, 98%, or 99% sequence identity therewith.

In some embodiments, the secondary structure comprises a quadruplex. In some embodiments, the secondary structure comprises a G-quadruplex. In some embodiments, the secondary structure comprises a nucleotide sequence selected from the group consisting of gq2(UGGUGGUGGUGGU) (SEQ ID No: 866), stk40(GGGACAGGGCAGGGACAGGG) (SEQ ID No: 867), apc2(GGGUCCGGGUCUGGGUCUGGG) (SEQ ID No: 868), stard3(GGGCAGGGUCUGGGCUGGG) (SEQ ID No: 869), tnsl(GGGCUGGGAUGGGAAAGGG) (SEQ ID No: 870), ceacam4(GGGCUCUGGGUGGGCCGGG) (SEQ ID No: 871), ercl(GGGCUGGGCUGGGCAGGG) (SEQ ID No: 872), pitpnm3(GGGUGGGCUGGGAAGGG) (SEQ ID No: 873), rlf(GGGAGGGAGGGCUAGGG) (SEQ ID No: 874), ube3c(GGGCAGGGCUGGGAGGG) (SEQ ID No: 875), taf15(GGGUGGGAGGGCUGGG) (SEQ ID No: 876), and xrnl(GCGUAACCUCCAUCCGAGUUGCAAGAGAGGGAAACGCAGUCUC) (SEQ ID No: 877), or a nucleotide sequence having at least 80%, 85%, 90%, 95%, 96%, 97%, 98%, or 99% sequence identity therewith.

In some embodiments, the secondary structure comprises aP4-P6 domain of a Group I intron. In some embodiments, the secondary structure comprises the nucleotide sequence of GGAAUUGCGGGAAAGGGGUCAACAGCCGUUCAGUACCAAGUCUCAGGGGAAACUUUG AGAUGGCCUUGCAAAGGGUAUGGUAAUAAGCUGACGGACAUGGUCCUAACCACGCAG CCAAGUCCUAAGUCAACAGAUCUUCUGUUGAUAUGGAUGCAGUUCA (SEQ ID No: 878), or a nucleotide sequence having at least 80%, 85%, 90%, 95%, 96%, 97%, 98%, or 99% sequence identity therewith.

In some embodiments, the secondary structure comprises a riboswitch aptamer. In some embodiments, the secondary structure comprises a riboswitch aptamer derived from a prequeosine-1 riboswitch aptamer. In some embodiments, the secondary structure comprises a modified prequeosine-1 riboswitch aptamer. In some embodiments, the secondary structure comprises a nucleotide sequence selected from the group consisting of UUGACGCGGUUCUAUCUAGUUACGCGUUAAACCAACUAGAAA (SEQ ID No: 879), UUGACGCGGUUCUAUCUACUUACGCGUUAAACCAACUAGAAA (SEQ ID No: 880), CGCGAGUCUAGGGGAUAACGCGUUAAACUUCCUAGAAGGCGGUU (SEQ ID No: 881), CGCGGAUCUAGAUUGUAACGCGUUAAACCAUCUAGAAGGCGGUU (SEQ ID No: 882), CGCGUCGCUACCGCCCGGCGCGUUAAACACACUAGAAGGCGGUU (SEQ ID No: 883), and CGCGGUUCUAUCUAGUUACGCGUUAAACCAACUAGAA (SEQ ID No: 885), or a nucleotide sequence having at least 80%, 85%, 90%, 95%, 96%, 97%, 98%, or 99% sequence identity therewith. In some embodiments, the secondary structure comprises a nucleotide sequence selected from the group consisting of UUGACGCGGUUCUAUCUAGUUACGCGUUAAACCAACUAGAAA (SEQ ID No: 879), CGCGAGUCUAGGGGAUAACGCGUUAAACUUCCUAGAAGGCGGUU (SEQ ID No: 881), CGCGGAUCUAGAUUGUAACGCGUUAAACCAUCUAGAAGGCGGUU (SEQ ID No: 882), CGCGUCGCUACCGCCCGGCGCGUUAAACACACUAGAAGGCGGUU (SEQ ID No: 883), and CGCGGUUCUAUCUAGUUACGCGUUAAACCAACUAGAA (SEQ ID No: 885), or a nucleotide sequence having at least 80%, 85%, 90%, 95%, 96%, 97%, 98%, or 99% sequence identity therewith. In some embodiments, the secondary structure comprises a nucleotide sequence of and CGCGGUUCUAUCUAGUUACGCGUUAAACCAACUAGAA (SEQ ID No: 885), or a nucleotide sequence having at least 80%, 85%, 90%, 95%, 96%, 97%, 98%, or 99% sequence identity therewith.

In some embodiments, the secondary structure is linked to one or more other component of a PEgRNA via a linker. For example, in some embodiments, the secondary structure is at the 3′ end of the PEgRNA and is linked to the 3′ end of a PBS via a linker. In some embodiments, the secondary structure is at the 5′ end of the PEgRNA and is linked to the 5′ end of a spacer via a linker. In some embodiments, the linker is a nucleotide linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the linker is 5 to 10 nucleotides in length. In some embodiments, the linker is 10 to 20 nucleotides in length. In some embodiments, the linker is 15 to 25 nucleotides in length. In some embodiments, the linker is 8 nucleotides in length.

In some embodiments, the linker is designed to minimize base pairing between the linker and another component of the PEgRNA. In some embodiments, the linker is designed to minimize base pairing between the linker and the spacer. In some embodiments, the linker is designed to minimize base pairing between the linker and the PBS. In some embodiments, the linker is designed to minimize base pairing between the linker and the editing template. In some embodiments, the linker is designed to minimize base pairing between the linker and the sequence of the RNA secondary structure. In some embodiments, the linker is optimized to minimize base pairing between the linker and another component of the PEgRNA, in order of the following priority: spacer, PBS, editing template and then scaffold. In some embodiments, base paring probability is calculated using ViennaRNA 2.0, as described in Lorenz, R. et al. ViennaRNA package 2.0. Algorithms Mol. Biol. 6, incorporated by reference in its entirety herein, under standard parameters (37° C., 1 M NaCl, 0.05 M MgCl2).

In some embodiments, the PEgRNA comprises a RNA secondary structure and/or a linker disclosed in Nelson et al. Engineered pegRNAs improve prime editing efficiency. Nat Biotechnol. (2021), the entirety of which is incorporated herein by reference.

In some embodiments, a PEgRNA is transcribed from a nucleotide encoding the PEgRNA, for example, a DNA plasmid encoding the PEgRNA. In some embodiments, the PEgRNA comprises a self-cleaving element. In some embodiments, the self-cleaving element improves transcription and/or processing of the PEgRNA when transcribed form the nucleotide encoding the PEgRNA. In some embodiments, the PEgRNA comprises a hairpin or a RNA quadruplex. In some embodiments, the PEgRNA comprises a self-cleaving ribozyme element, for example, a hammerhead, a pistol, a hatchet, a hairpin, a VS, a twister, or a twister sister ribozyme. In some embodiments, the PEgRNA comprises a HDV ribozyme. In some embodiments, the PEgRNA comprises a hairpin recognized by Csy4. In some embodiments, the PEgRNA comprises an ENE motif. In some embodiments, the PEgRNA comprises an element for nuclear expression (ENE) from MALAT1 lnc RNA. In some embodiments, the PEgRNA comprises an ENE element from Kaposi's sarcoma-associated herpesvirus (KSHV). In some embodiments, the PEgRNA comprises a 3′ box of a Ul snRNA. In some embodiments, the PEgRNA forms a circular RNA.

In some embodiments, the PEgRNA comprises an RNA secondary structure or a motif that improves binding to the DNA-RNA duple or enhances PEgRNA activity. In some embodiments, the PEgRNA comprises a sequence derived from a native nucleotide element involved in reverse transcription, e.g., initiation of retroviral transcription. In some embodiments, the PEgRNA comprises a sequence of, or derived from, a primer binding site of a substrate of a reverse transcriptase, a polypurine tract (PPT), or a kissing loop. In some embodiments, the PEgRNA comprises a dimerization motif, a kissing loop, or a GNRA tetraloop—tetraloop receptor pair that results in circularization of the PEgRNA. In some embodiments, the PEgRNA comprises an RNA secondary structure of a motif that results in physical separation of the spacer and the PBS of the PEgRNA, thereby prevents occlusion of the spacer and improves PEgRNA activity. In some embodiments, the PEgRNA comprises a secondary structure or motif, e.g., a 5′ or 3′ extension in the spacer region that form a toehold or hairpin, wherein the secondary structure or motif competes favorably against annealing between the spacer and the PBS of the PEgRNA, thereby prevents occlusion of the spacer and improves PEgRNA activity.

In some embodiments, a PEgRNA comprises the sequence GGCCGGCAUGGUCCCAGCCUCCUCGCUGGCGCCGGCUGGGCAACAUGCUUCGGCAUG GCGAAUGGGAC (SEQ ID No: 886) at the 3′ end. In some embodiments, a PEgRNA comprises the structure [spacer]-[gRNA core]-[editing template]-[PBS]-GGCCGGCAUGGUCCCAGCCUCCUCGCUGGCGCCGGCUGGGCAACAUGCUUCGGCAUG GCGAAUGGGAC (SEQ ID NO: 886), or [spacer]-[gRNA core]-[editing template]-[PBS]-GGCC GGCAUGGUCCCAGCCUCCUC GCUGGC GCC GGCUGGGCAACAUGCUUC GGCAUG GCGAAUGGGAC-(U)n (SEQ ID NO: 900), wherein n is an integer between 3 and 7. The structure derived from hepatitis D virus (HDV) is italicized.

In some embodiments, the PEgRNA comprises the sequence GGUGGGAGACGUCCCACC (SEQ ID No: 880) at the 5′ end and/or the sequence UGGGAGACGUCCCACC (SEQ ID NO: 901) at the 3′ end. In some embodiments, the PEgRNA comprises the following structure (M-MLV kissing loop): GGUGGGAGACGUCCCACC (SEQ ID NO: 880)-[spacer]-[gRNA core]-[editing template]-[PBS]-UGGGAGACGUCCCACC (SEQ ID NO: 901), or GGUGGGAGACGUCCCACC (SEQ ID NO: 880)-[spacer]-[gRNA core]-[editing template]-[PBS]-UGGGAGACGUCCCACC-(U)n (SEQ ID NO: 902), wherein n is an integer between 3 and 7. The kissing loop structure is italicized.

In some embodiments, the PEgRNA comprises the sequence GAGCAGCAUGGCGUCGCUGCUCAC (SEQ ID No: 888) at the 5′ end and/or the sequence CCAUCAGUUGACACCCUGAGG (SEQ ID No: 889) at the 3′ end. In some embodiments, the PEgRNA comprises the following structure (VS ribozyme kissing loop):

GAGCAGCAUGGCGUCGCUGCUCAC (SEQ ID NO: 888)-[spacer]-[gRNA core]-[editing template]-[PBS]- CCAUCAGUUGACACCCUGAGG (SEQ ID NO: 889s), or GAGCAGCAUGGCGUCGCUGCUCAC (SEQ ID NO: 888)-[spacer]-[gRNA core]-[editing template]-[PBS]- CCAUCAGUUGACACCCUGAGG-(U)n (SEQ ID NO: 903), wherein n is an integer between 3 and 7. (VS ribozyme kissing loop)

In some embodiments, the PEgRNA comprises the sequence GCAGACCUAAGUGGUGACAUAUGGUCUG (SEQ ID No: 890) at the 5′ end and/or the sequence CAUGCGAUUAGAAAUAAUCGCAUG (SEQ ID No: 891) at the 3′ end. In some embodiments, the PEgRNA comprises the following structure (tetraloop and receptor): GCAGACCUAAGUGGUGACAUAUGGUCUG (SEQ ID NO: 890)-[spacer]-[gRNA core]-[editing template]-[PBS]- CAUGCGAUUAGAAAUAAUCGCAUG (SEQ ID NO: 891), or GCAGACCUAAGUGGUGACAUAUGGUCUG (SEQ ID NO: 890)-[spacer]-[gRNA core]-[editing template]-[PBS]- CAUGCGAUUAGAAAUAAUCGCAUG-(U)n (SEQ ID NO: 904), wherein n is an integer between 3 and 7. The tetraloop/tetraloop receptor structure is italicized.

In some embodiments, the PEgRNA comprises the sequence GGCCGGCAUGGUCCCAGCCUCCUCGCUGGCGCCGGCUGGGCAACAUGCUUCGGCAUG GCGAAUGGGAC (SEQ ID No: 886) or UCUGCCAUCAAAGCUGCGACCGUGCUCAGUCUGGUGGGAGACGUCCCACCGGCCGGC AUGGUCCCAGCCUCCUCGCUGGCGCCGGCUGGGCAACAUGCUUCGGCAUGGCGAAUG GGAC (SEQ ID No: 892) at the 3′ end.

In some embodiments, a PEgRNA comprises a gRNA core that comprises a modified direct repeat compared to the sequence of a naturally occurring CRISPR-Cas guide RNA scaffold, for example, a Cas9 gRNA scaffold. In some embodiments, the PEgRNA comprises a “flip and extension (F+E)” gRNA core, wherein one or more base pairs in a direct repeat is modified. In some embodiments, the PEgRNA comprises a first direct repeat (the first paring element or the lower stem), wherein a Uracil is changed to a Adenine (such that in the stem region, a U-A base pair is changed to a A-U base pair). In some embodiments, the PEgRNA comprises a first direct repeat wherein the fourth U-A base pair in the stem is changed to a A-U base pair. In some embodiments, the PEgRNA comprises a first direct repeat wherein one or more U-A base pair is changed to a G-C or C-G base pair. For example, in some embodiments, the PEgRNA comprises a first direct repeat comprising a modification to a GUUUU-AAAAC pairing element, wherein one or more of the U-A base pairs is changed to a A-U base pair, a G-C base pair, or a C-G base pair. In some embodiments, the PEgRNA comprises an extended first direct repeat.

In some embodiments, a PEgRNA comprises a gRNA core comprises the sequence

(SEQ ID No: 893)
GUUUUAGAGCUAUACGUAGCAAGUUAAAAUAAGGCUAGUCCGUUAUCAA
CUUUACGAAGUGGCACCGAGUCGGUGC
or
(SEQ ID NO: 894)
GUUUUAGAGCUAUACGUAGCAAGUUAAAAUAAGGCUAGUCCGUUAUCAA
CUUUACGAAGUGGGACCGAGUCGGUCC.

In some embodiments, a PEgRNA comprises a gRNA core comprising the sequence

(SEQ ID NO: 895)
GUUUUAGAGCUAGCUCAUGAAAAUGAGCUAGCAAGUUAAAAUAAGGCUA
GUCCGUUAUCAACUUGAAAAAGUGGGACCGAGUCGGUCC.

In some embodiments, a PEgRNA comprises a gRNA core comprising the sequence

(SEQ ID NO: 896)
GUUUGAGAGCUAGAAAUAGCAAGUUUAAAUAAGGCUAGUCCGUUAUCAA
CUUGAAAAAGUGGGACCGAGUCGGUCC.

In some embodiments, a PEgRNA comprises a gRNA core comprising the sequence

GUUUAAGAGCUAGAAAUAGCAAGUUUAAAUAAGGCUAGUCCGUUAUCAACUUGA AAAAGUGGCACCGAGUCGGUGC (SEQ ID No: 897). In some embodiments, a PEgRNA comprise a gRNA core comprising the sequence

(SEQ ID NO: 898)
GUUUUAGAGCUAUGCUGGAAACAGCAUAGCAAGUUAAAAUAAGGCUAGU
CCGUUAUCAACUUGAAAAAGUGGCACCGAGUCGGUGC.

In some embodiments, a PEgRNA comprise a gRNA core comprising the sequence

(SEQ ID NO: 899)
GUUUAAGAGCUAUGCUGGAAACAGCAUAGCAAGUUUAAAUAAGGCUAGU
CCGUUAUCAACUUGAAAAAGUGGCACCG AGU CGGUGC.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in the disclosure may further comprise nucleotides added to the 5′ of the PEgRNAs. In some embodiments, the PEgRNA further comprises 1, 2, or 3 additional nucleotides added to the 5′ end. The additional nucleotides can be guanine, cytosine, adenine, or uracil. In some embodiments, the additional nucleotide at the 5′ end of the PEgRNA is a guanine or cytosine. In some embodiments, the additional nucleotides can be chemically or biologically modified.

In some embodiments, the PEgRNAs provided in the disclosure may further comprise nucleotides to the 3′ of the PEgRNAs. In some embodiments, the PEgRNA further comprises 1, 2, or 3 additional nucleotides to the 3′ end. The additional nucleotides can be guanine, cytosine, adenine, or uracil. In some embodiments, the additional nucleotides at the 3′ end of the PEgRNA is a polynucleotide comprising at least 1 uracil. In some embodiments, the additional nucleotides can be chemically or biologically modified.

In some embodiments, a PEgRNA or ngRNA is produced by transcription from a template nucleotide, for example, a template plasmid. In some embodiments, a polynucleotide encoding the PEgRNA or ngRNA is appended with one or more additional nucleotides that improves PEgRNA or ngRNA function or expression, e.g., expression from a plasmid that encodes the PEgRNA or ngRNA. In some embodiments, a polynucleotide encoding a PEgRNA or ngRNA is appended with one or more additional nucleotides at the 5′ end or at the 3′ end. In some embodiments, the polynucleotide encoding the PEgRNA or ngRNA is appended with a guanine at the 5′ end, for example, if the first nucleotide at the 5′ end of the spacer is not a guanine. In some embodiments, a polynucleotide encoding the PEgRNA or ngRNA is appended with nucleotide sequence CACC at the 5′ end. In some embodiments, the polynucleotide encoding the PEgRNA or ngRNA is appended with an additional nucleotide adenine at the 3′ end, for example, if the last nucleotide at the 3′ end of the PBS is a Thymine. In some embodiments, the polynucleotide encoding the PEgRNA or ngRNA is appended with additional nucleotide sequence TTTTTT (SEQ ID NO: 92445), TTTTTTT (SEQ ID NO: 92446), TTTTT (SEQ ID NO:), or TTTT (SEQ ID NO:) at the 3′ end. In some embodiments, the PEgRNA or ngRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises an adenine at the 3′ end, for example, if the last nucleotide at the 3′ end of the PBS is a thymine. In some embodiments, the PEgRNA or ngRNA further comprises nucleotide sequence UUUUUUU (SEQ ID NO: 92448), UUUUUU (SEQ ID NO: 92447), UUUUU (SEQ ID NO:), or UUUU (SEQ ID NO:) at the 3′ end.

In some embodiments, the PEgRNAs and/or ngRNAs provided in this disclosure may have undergone a chemical or biological modification. Modifications may be made at any position within a PEgRNA or ngRNA and may include modification to a nucleobase or to a phosphate backbone of the PEgRNA or ngRNA. In some embodiments, chemical modifications can be structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification can be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification can be within the 3′ most nucleotides of a PEgRNA or ngRNA. In some embodiments, a chemical modification can be within the 3′ most end of a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a chemical modification can be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. The gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′ phosphorothioate (MS), or a 2′-O-methyl 3′ thioPACE (MSP), or any combination thereof. In some embodiments, a chemically modified PEgRNA and/or ngRNA can comprise a′-O-methyl (M) RNA, a 2′-O-methyl 3′ phosphorothioate (MS) RNA, a 2′-O-methyl 3′ thioPACE (MSP) RNA, a 2′-F RNA, a phosphorothioate bond modification, any other chemical modifications known in the art, or any combination thereof. A chemical modification can also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor provided herein and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA may be delivered simultaneously. For example, in some embodiments, a polynucleotide encoding the prime editor and a polynucleotide encoding a PEgRNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

### Pharmaceutical Compositions

Other aspects of the present disclosure relate to pharmaceutical compositions comprising any of the prime editors, fusion proteins, polynucleotides encoding prime editor polypeptides, PEgRNAs, ngRNAs, prime editing complexes, and/or the fusion protein-guide polynucleotide complexes described herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds). As used here, the term “pharmaceutically-acceptable carrier” means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanthin; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier,” “pharmaceutically acceptable carrier” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to, salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

The pharmaceutical composition comprising any of the prime editors, fusion proteins, polynucleotides encoding prime editor polypeptides, PEgRNAs, ngRNAs, prime editing complexes, and/or the fusion protein-guide polynucleotide complexes described herein may be used in vitro or in vivo. In some embodiments, the pharmaceutical composition or any components thereof are delivered to cells (e.g., mammalian cells, e.g., human cells), e.g., in vitro or in vivo. In some embodiments, the pharmaceutical composition is formulated for delivery to a subject, e.g., for prime editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber. In other embodiments, the pharmaceutical composition described herein is delivered in a controlled release system. In some embodiments, a pump can be used. In some embodiments, polymeric materials can be used. In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in“stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating. Positively charged lipids such as N-[1-(2,3-dioleoyloxi)propyl]-N,N,N-trimethyl-amoniummethylsulfate, or “DOTAP,” are particularly preferred for such particles and vesicles.

The pharmaceutical composition described herein can be administered or packaged as a unit dose, for example, in reference to a pharmaceutical composition to physically discrete units suitable as unitary dosage for the subject, each unit containing a predetermined quantity of active material calculated to produce the desired therapeutic effect in association with the required diluent, i.e., carrier, or vehicle. Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Any of the prime editors, fusion proteins, PEgRNAs, and/or complexes described herein are provided as part of a pharmaceutical composition. In some embodiments, the pharmaceutical composition comprises any of the prime editors or fusion proteins provided herein. In some embodiments, the pharmaceutical composition comprises any of the complexes provided herein. In some embodiments, the pharmaceutical composition comprises a ribonucleoprotein complex comprising a prime editor that forms a complex with a PEgRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

### Methods of Editing

The methods and compositions disclosed herein can be used to edit a target gene of interest by prime editing. In some embodiments, the prime editing method comprises contacting a target gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, upon the contacting of the PE composition with the target gene. In some embodiments, the DNA binding domain of the PE associates with the PEgRNA. In some embodiments, the PE binds the target gene, directed by the PEgRNA. Accordingly, in some embodiments, the contacting of the target gene result in binding of a DNA binding domain of a prime editor of the target gene directed by the PEgRNA.

In some embodiments, contacting the target gene with the prime editing composition results in a nick in an edit strand of the target gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene, is in a cell. Accordingly, also provided herein are methods of modifying a cell. Accordingly, also provided herein are methods of modifying a cell, such as a human cell, a human primary cell, a human iPSC-derived cell, and human HSPC.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell.

In some embodiments, the cell is a primary cell. In some embodiments, the cell is a human primary cell. In some embodiments, the cell is a progenitor cell. In some embodiments, the cell is a human progenitor cell. In some embodiments, the cell is a human cell from an organ. In some embodiments, the cell is a primary human cell derived from an induced human pluripotent stem cell (iPSC). In some embodiments, the cell is a hepatocyte. In some embodiments, the cell is a human HSPC. In some embodiments, the cell is a progenitor cell. In some embodiments, the cell is a stem cell. in some embodiments, the cell is an induced pluripotent stem cell. In some embodiments, the cell is an embryonic stem cell. In some embodiments, the cell is a hematopoietic progenitor cell. In some embodiments, the cell is a hematopoietic stem cell. In some embodiments, the cell is a hematopoietic stem or progenitor cell (HSPC). In some embodiments, the cell is a fibroblast. In some embodiments, the cell is a CD34+ cell. In some embodiments, the cell is a hematopoietic stem cell (HSC). In some embodiments, the cell is a hematopoietic progenitor cell (HPC). In some embodiments, the cell is a human HSPC. In some embodiments, the cell is a human HPC. In some embodiments, the cell is a human HSC. In some embodiments, the cell is a long term (LT)-HSC. In some embodiments, the cell is a short-term(ST)-HSC. In some embodiments, the cell is a myeloid progenitor cell. In some embodiments, the cell is a lymphoid progenitor cell. In some embodiments, the cell is a granulocyte monocyte progenitor cell. In some embodiments, the cell is a megakaryocyte erythroid progenitor cell.

In some embodiments, the cell is a human progenitor cell. In some embodiments, the cell is a human stem cell. in some embodiments, the cell is an induced human pluripotent stem cell. In some embodiments, the cell is a human embryonic stem cell. In some embodiments, the cell is a human fibroblast. In some embodiments, the cell is a primary cell. In some embodiments, the cell is a human primary cell. In some embodiments, the cell is a differentiated cell. In some embodiments, the cell edited by prime editing can be differentiated into or give rise to recovery of a population of cells, e.g., neutrophils, platelets, red blood cells, monocytes, macrophages, antigen-presenting cells, microglia, osteoclasts, dendritic cells, inner ear cell, inner ear support cell, cochlear cell and/or lymphocytes.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a target gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. Prime editing methods disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a target cell, e.g., a primary cell, relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 1% in a target cell, e.g., a human primary cell or hepatocyte. In some embodiments, the prime editing methods disclosed herein have an editing efficiency of at least about 1% and an indel frequency of less than 0.5% in a target cell, e.g., a human primary cell or hepatocyte. In some embodiments, the prime editing methods disclosed herein have an editing efficiency of at least about 1% and an indel frequency of less than 0.1% in a target cell, e.g., a human primary cell or hepatocyte. In some embodiments, the prime editing methods disclosed herein have an editing efficiency of at least about 5% and an indel frequency of less than 1% in a target cell, e.g., a human primary cell or hepatocyte. In some embodiments, the prime editing methods disclosed herein have an editing efficiency of at least about 5% and an indel frequency of less than 0.5% in a target cell, e.g., a human primary cell or hepatocyte. In some embodiments, the prime editing methods disclosed herein have an editing efficiency of at least about 5% and an indel frequency of less than 0.1% in a target cell, e.g., a human primary cell.

In some embodiments, the prime editing methods disclosed herein have an editing efficiency of at least about 7.5% and an indel frequency of less than 1% in a target cell, e.g., a human primary cell or hepatocyte. In some embodiments, the prime editing methods disclosed herein have an editing efficiency of at least about 7.5% and an indel frequency of less than 0.5% in a target cell, e.g., a human primary cell or hepatocyte. In some embodiments, the prime editing methods disclosed herein have an editing efficiency of at least about 7.5% and an indel frequency of less than 0.1% in a target cell, e.g., a human primary cell or hepatocyte.

In some embodiments, the prime editing methods disclosed herein have an editing efficiency of at least about 10% and an indel frequency of less than 1% in a target cell, e.g., a human primary cell or hepatocyte. In some embodiments, the prime editing methods disclosed herein have an editing efficiency of at least about 10% and an indel frequency of less than 0.5% in a target cell, e.g., a human primary cell or hepatocyte. In some embodiments, the prime editing methods disclosed herein have an editing efficiency of at least about 10% and an indel frequency of less than 0.1% in a target cell, e.g., a human primary cell or hepatocyte.

In some embodiments, the prime editing methods disclosed herein have an editing efficiency of at least about 15% and an indel frequency of less than 1% in a target cell, e.g., a human primary cell or hepatocyte. In some embodiments, the prime editing methods disclosed herein have an editing efficiency of at least about 15% and an indel frequency of less than 0.5% in a target cell, e.g., a human primary cell or hepatocyte. In some embodiments, the prime editing methods disclosed herein have an editing efficiency of at least about 15% and an indel frequency of less than 0.1% in a target cell, e.g., a human primary cell or hepatocyte.

In some embodiments, the prime editing methods disclosed herein have an editing efficiency of at least about 20% and an indel frequency of less than 1% in a target cell, e.g., a human primary cell or hepatocyte. In some embodiments, the prime editing methods disclosed herein have an editing efficiency of at least about 20% and an indel frequency of less than 0.5% in a target cell, e.g., a human primary cell or hepatocyte. In some embodiments, the prime editing methods disclosed herein have an editing efficiency of at least about 20% and an indel frequency of less than 0.1% in a target cell, e.g., a human primary cell or hepatocyte.

In some embodiments, the prime editing methods disclosed herein have an editing efficiency of at least about 30% and an indel frequency of less than 1% in a target cell, e.g., a human primary cell or hepatocyte. In some embodiments, the prime editing methods disclosed herein have an editing efficiency of at least about 30% and an indel frequency of less than 0.5% in a target cell, e.g., a human primary cell or hepatocyte. In some embodiments, the prime editing methods disclosed herein have an editing efficiency of at least about 30% and an indel frequency of less than 0.1% in a target cell, e.g., a human primary cell or hepatocyte.

In some embodiments, the prime editing methods disclosed herein have an editing efficiency of at least about 40% and an indel frequency of less than 1% in a target cell, e.g., a human primary cell or hepatocyte. In some embodiments, the prime editing methods disclosed herein have an editing efficiency of at least about 40% and an indel frequency of less than 0.5% in a target cell, e.g., a human primary cell or hepatocyte. In some embodiments, the prime editing methods disclosed herein have an editing efficiency of at least about 40% and an indel frequency of less than 0.1% in a target cell, e.g., a human primary cell or hepatocyte.

In some embodiments, the prime editing methods disclosed herein have an editing efficiency of at least about 50% and an indel frequency of less than 1% in a target cell, e.g., a human primary cell or hepatocyte. In some embodiments, the prime editing methods disclosed herein have an editing efficiency of at least about 50% and an indel frequency of less than 0.5% in a target cell, e.g., a human primary cell or hepatocyte. In some embodiments, the prime editing methods disclosed herein have an editing efficiency of at least about 50% and an indel frequency of less than 0.1% in a target cell, e.g., a human primary cell or hepatocyte.

In some embodiments, the prime editing methods disclosed herein have an editing efficiency of at least about 60% and an indel frequency of less than 1% in a target cell, e.g., a human primary cell or hepatocyte. In some embodiments, the prime editing methods disclosed herein have an editing efficiency of at least about 60% and an indel frequency of less than 0.5% in a target cell, e.g., a human primary cell or hepatocyte. In some embodiments, the prime editing methods disclosed herein have an editing efficiency of at least about 60% and an indel frequency of less than 0.1% in a target cell, e.g., a human primary cell or hepatocyte.

In some embodiments, the prime editing methods disclosed herein have an editing efficiency of at least about 70% and an indel frequency of less than 1% in a target cell, e.g., a human primary cell or hepatocyte. In some embodiments, the prime editing methods disclosed herein have an editing efficiency of at least about 70% and an indel frequency of less than 0.5% in a target cell, e.g., a human primary cell or hepatocyte. In some embodiments, the prime editing methods disclosed herein have an editing efficiency of at least about 70% and an indel frequency of less than 0.1% in a target cell, e.g., a human primary cell or hepatocyte.

In some embodiments, the prime editing methods disclosed herein have an editing efficiency of at least about 80% and an indel frequency of less than 1% in a target cell, e.g., a human primary cell or hepatocyte. In some embodiments, the prime editing methods disclosed herein have an editing efficiency of at least about 80% and an indel frequency of less than 0.5% in a target cell, e.g., a human primary cell or hepatocyte. In some embodiments, the prime editing methods disclosed herein have an editing efficiency of at least about 80% and an indel frequency of less than 0.1% in a target cell, e.g., a human primary cell or hepatocyte.

In some embodiments, the prime editing methods disclosed herein have an editing efficiency of at least about 90% and an indel frequency of less than 1% in a target cell, e.g., a human primary cell or hepatocyte. In some embodiments, the prime editing methods disclosed herein have an editing efficiency of at least about 90% and an indel frequency of less than 0.5% in a target cell, e.g., a human primary cell or hepatocyte. In some embodiments, the prime editing methods disclosed herein have an editing efficiency of at least about 90% and an indel frequency of less than 0.1% in a target cell, e.g., a human primary cell or hepatocyte.

In some embodiments, the prime editing methods disclosed herein have an editing efficiency of at least about 95% and an indel frequency of less than 1% in a target cell, e.g., a human primary cell or hepatocyte. In some embodiments, the prime editing methods disclosed herein have an editing efficiency of at least about 95% and an indel frequency of less than 0.5% in a target cell, e.g., a human primary cell or hepatocyte. In some embodiments, the prime editing methods disclosed herein have an editing efficiency of at least about 95% and an indel frequency of less than 0.1% in a target cell, e.g., a human primary cell or hepatocyte. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and prime editors as described herein) and prime editing methods disclosed herein can be used to edit a target gene. In some embodiments, the target gene comprises a mutation compared to a wild type gene. In some embodiments, the mutation is associated a disease. In some embodiments, the target gene comprises an editing target sequence that contains the mutation associated with a disease. In some embodiments, the mutation is in a coding region of the target gene. In some embodiments, the mutation is in an exon of the target gene. In some embodiments, the prime editing method comprises contacting a target gene with a prime editing composition comprising a prime editor, a PEgRNA, and/or a ngRNA. In some embodiments, contacting the target gene with the prime editing composition results in incorporation of one or more intended nucleotide edits in the target gene. In some embodiments, the incorporation is in a region of the target gene that corresponds to an editing target sequence in the gene. In some embodiments, the one or more intended nucleotide edits comprises a single nucleotide substitution, an insertion, a deletion, or any combination thereof, compared to the endogenous sequence of the target gene. In some embodiments, incorporation of the one or more intended nucleotide edits results in replacement of one or more mutations with the corresponding sequence that encodes a wild type protein. In some embodiments, incorporation of the one or more intended nucleotide edits results in replacement of the one or more mutations with the corresponding sequence in a wild type gene. In some embodiments, incorporation of the one more intended nucleotide edits results in correction of a mutation in the target gene. In some embodiments, the target gene comprises an editing template sequence that contains the mutation. In some embodiments, contacting the target gene with the prime editing composition results in incorporation of one or more intended nucleotide edits in the target gene, which corrects the mutation in the editing target sequence (or a double stranded region comprising the editing target sequence and the complementary sequence to the editing target sequence on a target strand) in the target gene.

In some embodiments, incorporation of the one more intended nucleotide edits results in correction of a mutation in. In some embodiments, incorporation of the one more intended nucleotide edits results in correction of an gene sequence and restores wild type expression and function of the protein.

In some embodiments, the target gene is in a target cell. Accordingly, in one aspect provided herein is a method of editing a target cell comprising a target gene that encodes a polypeptide that comprises one or more mutations relative to a wild type gene. In some embodiments, the methods of the present disclosure comprise introducing a prime editing composition comprising a PEgRNA, a prime editor polypeptide, and/or a ngRNA into the target cell that has the target gene to edit the target gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a hepatocyte. In some embodiments, the target cell is a human hepatocyte. In some embodiments, the target cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target gene that comprises one or more mutations restores wild type expression and function of protein encoded by the gene. In some embodiments, the target gene encodes at least one mutation as compared to the wild type protein prior to incorporation of the one or more intended nucleotide edits. In some embodiments, expression and/or function of protein may be measured when expressed in a target cell. In some embodiments, incorporation of the one or more intended nucleotide edits in the target gene comprising one or more mutations lead to a fold change in a level of gene expression, protein expression, or a combination thereof. In some embodiments, a change in the level of gene expression can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target gene that comprises one or more mutations restores wild type expression of protein by at least 10%, 20%, 30%, 40%, 50%, 60%, 70%, 80%, 90%, 95%, 99% or more as compared to wild type expression of the protein in a suitable control cell that comprises a wild type gene.

In some embodiments, an expression increase can be measured by a functional assay. In some embodiments, protein expression can be measured using a protein assay. In some embodiments, protein expression can be measured using antibody testing. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

Methods of Treating Diseases Associated with Pathological Mutations

Provided also are methods of treating a disease or disorder that involve the introduction of a prime editor into a disease-associated or disease-causing gene, or into a regulatory sequence (e.g., a gene promoter, enhancer, or repressor) associated with, for example, a gene having a mutation.

The method comprises administering to a subject (e.g., a mammal, such as a human) a therapeutically effective amount of a pharmaceutical composition that comprises a polynucleotide encoding a prime editor system (e.g., prime editor and PEgRNA) described herein. In some embodiments, the prime editor is a fusion protein that comprises a polynucleotide programmable DNA binding domain and an adenosine deaminase domain or a cytidine deaminase domain. A cell of the subject is transduced with the prime editor and one or more PEgRNA guide polynucleotides that direct the prime editor to effect a desired nucleotide edit in a disease-associated gene, a disease-causing gene, or a regulatory nucleic acid sequence associated with a disease-causing gene. For example, the desired nucleotide edit effected by prime editing my correct a disease associated mutation in the disease causing gene to a wild type gene sequence.

The methods herein include administering to the subject (including a subject identified as being in need of such treatment, or a subject suspected of being at risk of disease and in need of such treatment) an effective amount of a composition described herein. Identifying a subject in need of such treatment can be in the judgment of a subject or a health care professional and can be subjective (e.g. opinion) or objective (e.g. measurable by a test or diagnostic method).

In some embodiments, cells are obtained from the subject and contacted with a pharmaceutical composition as provided herein. In some embodiments, cells removed from a subject and contacted ex vivo with a pharmaceutical composition are re-introduced into the subject, optionally after the desired genomic modification has been effected by the prime editor or detected in the cells. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Disclosed herein, are prime editors, engineered RTs, and engineered Cas-RTs. Also disclosed are engineered RTs and engineered Cas-RTs. The engineered RTs and the engineered Cas-RTs may comprise amino acid or sequence variations of naturally occurring RTs and Cas-RTs. In some cases, the prime editors, engineered RTs, or engineered Cas-RTs may also comprise heterologous functional domains comprising SET domains. Other functional domains may comprise nuclear localization signals or sequences or linkers. In some instances, the prime editors, engineered RTs, or engineered Cas-RTs may complex with PEgRNAs. In some cases, prime editing may comprise the prime editors, engineered RTs, engineered Cas-RTs, or PEgRNAs. In some cases, a cell may comprise the prime editors, engineered RTs, engineered Cas-RTs, PEgRNAs, any derivatives thereof, or any combinations thereof. In some cases, a composition may comprise the prime editors, engineered RTs, engineered Cas-RTs, PEgRNAs, any derivatives thereof, any combinations thereof, or the cells comprising thereof. In some cases, a method for installing a nucleotide edit may comprise the prime editors, engineered RTs, engineered Cas-RTs, or PEgRNAs. In some cases, a method for treating a disorder may comprise the prime editors, engineered RTs, engineered Cas-RTs, PEgRNAs, any derivatives thereof, any combinations thereof cells comprising thereof, compositions comprising thereof, or kits comprising thereof. Prime editing, the methods for installing a nucleotide edit, the methods for treating a disorder, the methods of reverse transcribing an RNA sequence using the prime editors, engineered RTs, engineered Cas-RTs, PEgRNAs, any derivatives thereof, or any combinations thereof cells comprising thereof are also illustrated in the examples described herein.

Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA. In some embodiments, components of a prime editor composition may be delivered as a combination of DNA and RNA. In some embodiments, components of a prime editor composition can be delivered as a combination of nucleic acid, e.g., DNA and/or RNA and protein.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, HI promoter).

In some embodiments, the polynucleotide encoding one or more prime editing composition components is a part of, or is encoded by, a vector, e.g., a plasmid or a virus. In some embodiments, the vector is a viral vector. In some embodiments, the vector is a non-viral vector. In some embodiments, delivery is in vivo, in vitro, ex vivo, or in situ.

Non-viral vector delivery systems may include DNA plasmids, RNA (e.g., a transcript of a vector described herein), virosome, viral like particle, naked nucleic acid, and nucleic acid complexed with a delivery vehicle, such as a liposome. In some embodiments, the polynucleotide is provided as an RNA, e.g., an mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, an mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used. In some embodiments, the compositions and systems described herein can be formulated in liposomes or other similar vesicles. Liposomes may be anionic, neutral or cationic.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo), or they can be used to treat cells in vitro, and the modified cells can optionally be administered (ex vivo). In some embodiments, the virus is selected from a group I virus (e.g., a dsDNA virus), group II virus (e.g., a ssDNA virus), group III virus (e.g., a dsRNA virus), group IV virus (e.g., a +ssRNA virus), group V virus (e.g., a -ssRNA virus), group VI virus (e.g., a ssRNA-RT virus), or a group VII virus (e.g., a dsDNA-RT virus).

In some embodiments, the viral vector is a retroviral, lentiviral, adenoviral, adeno-associated viral, or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector. In some embodiments, the AAV is a recombinant AAV (rAAV).

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and kv2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiments, the AAV vector is selected for tropism to a particular cell, tissue, organism. In some embodiments, the AAV vector is pseudotyped, e.g., AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. Ii some embodiments, intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins. In some embodiments, the in vivo use of dual AAV vectors results in the expression of full-length full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. In some embodiments, insertion of an intein disrupts a Cas activity. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c. a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Can protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gRNA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK(SEQ ID NO: 936). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein (SEQ ID NO: 937), nona-arginine, and octa-arginine(SEQ ID NO: 938). The nona-arginine (R9) sequence(SEQ ID NO: 937) can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP), (e.g., a cationic lipid nanoparticle, an ionizable lipid nanoparticle), a micelle, polymer nanoparticle, Lipid—polymer nanoparticles (PLNs), or a combination thereof.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, a lipid nanoparticle may comprise a conjugated lipid, e.g., a PEG-phospholipid. Lipid nanoparticles may include additional elements, e.g., a polymer. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. In some embodiments, the lipid particle comprises a cationic lipid, a non-cationic lipid, a conjugated lipid that inhibits aggregation of particles, and/or a sterol. Exemplary lipids used to produce LNPs are provided in Tables 18 and 19 below. In some embodiments, a cationic lipid may be an ionizable cationic lipid, e.g., a cationic lipid that may carry a positive charge or be neutral depending on pH, or an amine-containing lipid that can be readily protonated. In some embodiments, a lipid nanoparticle may comprise a second cationic lipid.

In some embodiment, a polynucleotide encoding a prime editor polypeptide component may be co-formulated with a cationic lipid. In some embodiments, the nucleic acid may be encapsulated in an LNP. In embodiments, the LNP formulation is biodegradable. In some embodiments, LNPs are directed to specific tissues e.g., by displaying biological ligands on the surface of LNPs to enhance interaction with cognate receptors. In some embodiments, all components of a prime editor may be delivered in a single LNP formulation. In some embodiments, components of a prime editor may be delivered by separate LNP formulations.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 18 and 19 below.

TABLE 18
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropy 1)-N,N-dimethy 1-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy 1)-2,4,6-trimethylpyridinium | 2Oc | Cationic |
| 2,3-Dioleyloxy-N-P(spenninecarboxamido-ethy 1J-N,Ndimethyl- | DOSPA | Cationic |
| 1-propanatninium trifluoroacetate | | |
| 1,2-Dioley 1-3-trimethylamtnonium-propane | DOPA | Cationic |
| N-(2-Hydroxyethyl)-N,N-dimethy 1-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy 1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecy1-3-tetradecylaminopropionamidine | diC14-amidine | Cationic |
| Octadecenolyoxy [ethyl-2-heptadeceny1-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley 1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 19 below.

TABLE 19
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(ß-aminoester) | |
| Poly(4-hydroxy-L-proline ester) | PHP |
| Poly(allylamine) | |
| Poly(α-[4-aminobuty1]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 20 below.

TABLE 20
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very Transient | NO | DNA |
| | Herpes Simplex | YES | Stable | NO | DNA |
| | Virus | | | | |
| Non-Viral | Cationic liposome | YES | Transient | Depends on | Nucleic acids |
| | | | | what is | and Proteins |
| | | | | delivered | |
| | Polymeric | YES | Transient | NO | Nucleic Acids |
| | Nanoparticles | | | | |
| Biological | Attenuated Bacteria | YES | Transient | NO | Nucleic Acids |
| Non-Viral | Engineered | YES | Transient | NO | Nucleic Acids |
| Delivery | Bacteriophages | | | | |
| Vehicles | Mammalian Virus- | YES | Transient | NO | Nucleic Acids |
| | like Particles | | | | |
| | Biological | YES | Transient | NO | Nucleic Acids |
| | liposomes: | | | | |
| | Erythrocyte Ghosts | | | | |
| | and Exosomes | | | | |

The prime editing compositions of the disclosure, whether introduced as polynucleotides or polypeptides, may be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

The disclosure is not limited to the embodiments described and exemplified above, but is capable of variation and modification within the scope of the appended claims.

### EXAMPLES

These examples are provided for illustrative purposes only and not to limit the scope of the claims provided herein.

### Example 1—General Material and Methods

pegRNA and ngRNA assembly: For each pegRNA or nicking guide RNA (ngRNA), oligos encoding a spacer, a gRNA scaffold, and an extension arm (for nicking guide RNA, a spacer and a gRNA scaffold) were ligated by Gibson assembly or Golden Gate assembly and cloned into a U6 expression plasmid as described in Anzalone et al., Nature.2019 December; 576(7785): 149-157. Sequences of pegRNAs and ngRNAs are provided in Table 17, where editing targets of each pegRNA or ngRNA are also indicated.

HEK293 Transfection: HEK293T cells were seeded on 48-well poly-D-lysine coated plates (Corn-ing). Between 16 and 24 h after seeding, cells are transfected at approximately 60% confluency with 1 μl lipofectamine 2000 (Thermo Fisher Scientific) according to the manufacturer's protocols and 750 ng plasmid that expressed prime editors and 250 ng plasmid that expressed PEgRNAs. Cells are cultured for 3 days following transfection, after which the medium was removed, the cells were washed with lx PBS solution (Thermo Fisher Scientific). 3 biological replicates for each site are collected and genomic DNA is extracted by the addition of 150 μl of freshly prepared lysis buffer (10 mM Tris-HCl, pH 7.5; 0.05% SDS; 25 μg/ml proteinase K (ThermoFisher Scien-tific)) directly into each well of the tissue culture plate.

Editing activity determination: The genomic DNA mixture is incubated at 37° C. for 1-2 h, followed by an 80° C. enzyme inactivation step for 30 mM. Amplicons of the target loci are generated, barcoded, and sequenced on a Miseq from Illumina. Percent editing at the target locus is determined with Crispresso2 (Clement, K. et al., “CRISPResso2 provides accurate and rapid genome editing sequence analysis.” 2019, Nat Biotechnol 37, 224-226).

### Example 2: Reverse Transcriptase Homolog Screen

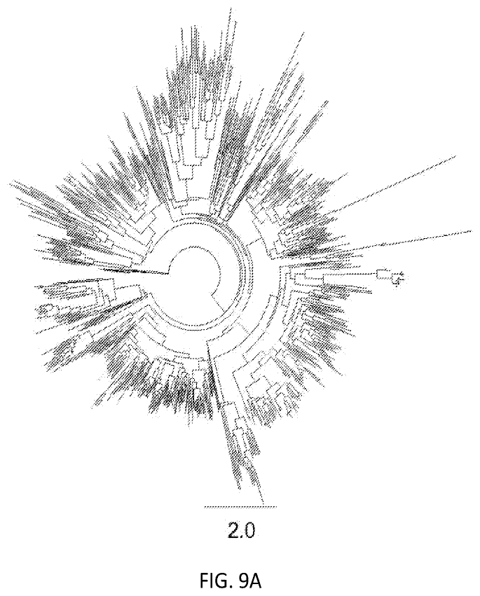

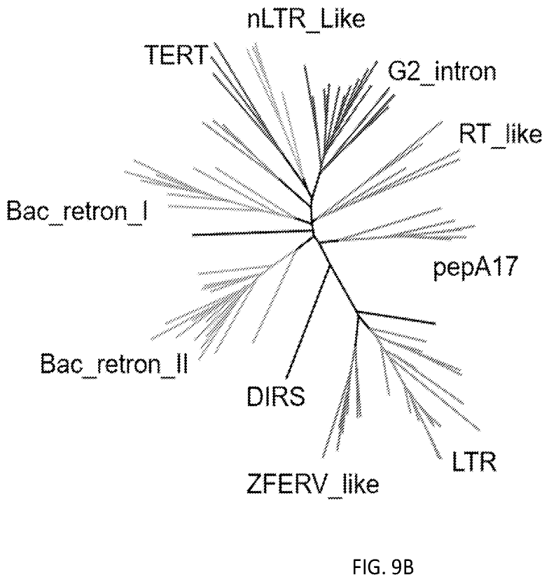

Reverse transcriptases (RTs) are abundant throughout eukaryotic and bacterial domains of life with over 600,000 reverse transcriptase sequences deposited in the NCBI. A hidden Markov model of evolutionary relationship (HMMER) model (See Eddy et al., “Profile hidden Markov models.” 1998, Bio-informatics 14, 755-763) was created by a search for homologous RT sequences from the National Center for Biotechnology Information (NCBI) and Uniprot sequence databases using 1305 viral RT sequences collected from the Uniprot viral proteome dataset as bait sequences. The search revealed 51,263 sequences with a significance bit score above 25.0 indicating likely homology to the input Viral RT sequences. Representative sequences from the full set of sequences were selected using the CD-hit2 (See Li, et al., “A. Cd-hit: a fast program for clustering and comparing large sets of protein or nucleotide sequences.” 2006, Bioinformatics 22, 1658-1659) software package with a cut-off of 80% sequence identity resulting in a set of 12,061 representative sequences. The resulting set of RT protein sequences was aligned with MAFFT3 sequence aligner. Following the initial alignment, the RT protein sequences were aligned an additional 499 times with stochastically selected alignment columns removed (bootstrapping) to identify areas of uncertainty in the alignment. The identified positions of uncertainty in the alignment were masked during further calculations. The masked protein sequence alignment was fitted to an evolutionary model using ModelFinder4 and a set of 350 evolutionary models. The best-fitting model, LG+R10+INV was used to infer a maxi-mum likelihood phylogenetic tree with 1000 bootstraps using the IQ-Tree5 software suite as shown in FIG. 9A. The resulting tree was searched for 89 sequences that best represent the topology of the tree and maximize the sequence diversity of RTs to place in an initial screen for PE activity, as illustrated in FIG. 9B. To identify additional RTs with Prime editing activity, the resulting tree identified above was further searched. These sequences were placed in an initial screen for prime editing efficiency.

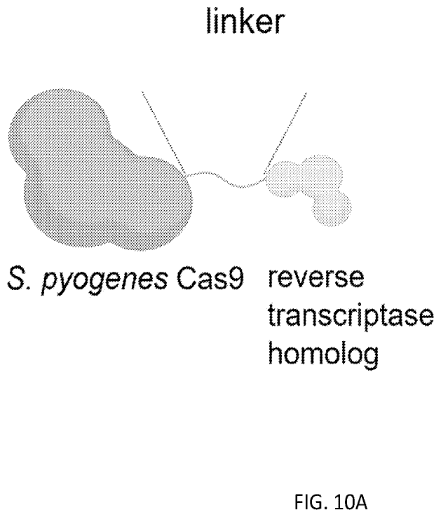

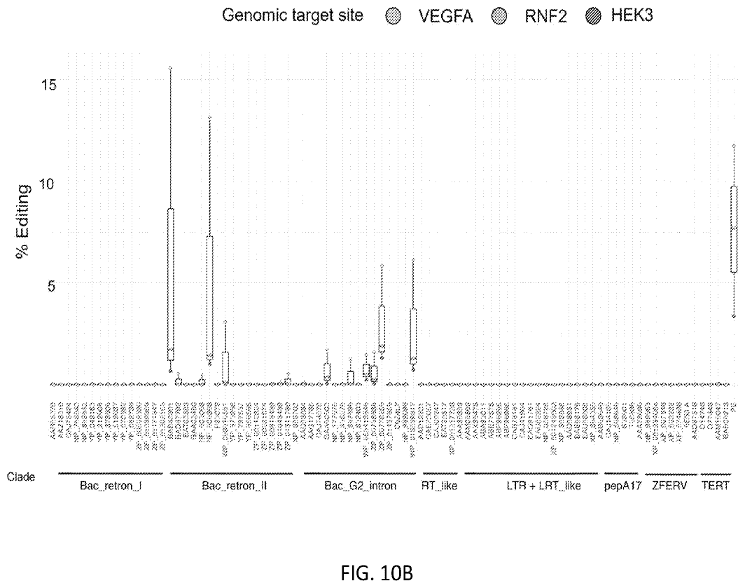

To screen for activity in the Prime Editor context, prime editors were constructed by replacing the MMLV-RT domain in the PE2 fusion protein (sequences shown in Table 15) with each of the identified reverse transcriptase domains. A simplified schematic of the prime editor configuration is provided in FIG. 10A. The resulting constructs were transfected into HEK293 cells along with corresponding plasmids encoding pegRNAs (and ngRNAs where applicable) targeting endogenous genetic loci using methods as described in Example 1 Initial screening results of editing efficiency targeting endogenous loci VEGFA, RNF2, and HEK3 are shown in FIG. 10B.

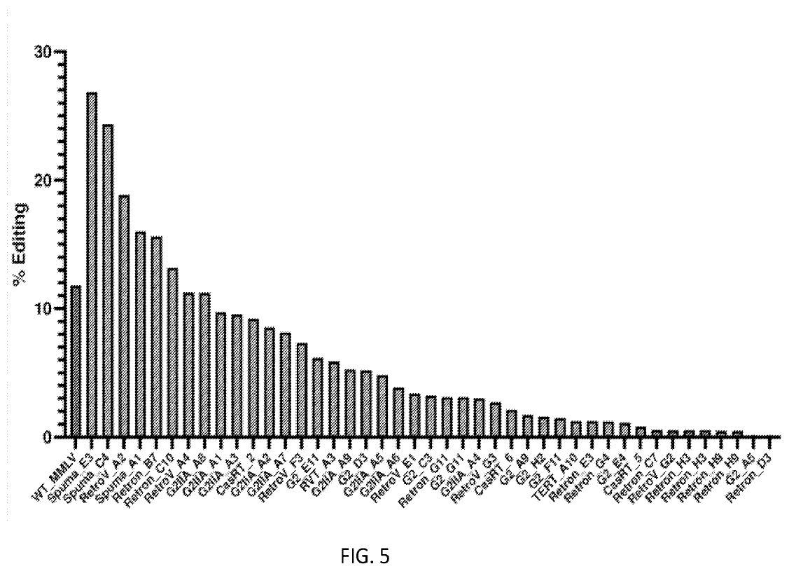

To further screen reverse transcriptase variants for Prime Editing activity, clades were selected from the full tree containing the representative sequences found to have Prime Editing activity in our initial survey screen. The resulting sequence list was used as inputs into a HMMER search using the HMM model described above to search three publicly available databases (NCBI, PATRIC, Uniprot). This sequence list was then passed through CD-HIT as described above to remove redundant sequences to a sequence identity of 85%. The resulting sequences were screened for prime editing activity as described above. Intermediate results of this screening at the VEGA locus are shown in FIG. 5. A set of RT homolog sequences tested in the Prime Editing context is provided in Table 1 and 2 (SEQ ID NO: 1-80, 198-271, 319-493, and 990-1006). Table 2 lists the reverse transcriptase homologs shown to have significant editing activity in the prime editing; editing efficiency at multiple genomic loci and construct sequences are reported. Except for the CasRT_2 and CasRT_6 constructs, which are discussed in Example 5, all the RT homologs in Table 2 were tested in Prime Editor constructs based on the PE2 design.

### Example 3—Ancestral Sequence Reconstruction (ASR) of the Zebrafish Endogenous Retrovirus (ZFERV) Subfamily of Reverse Transcriptase

ASR is a method by which a phylogenetic tree, a model of evolution, and a sequence alignment are used to infer the most likely sequences at nodes in the input phylogenetic tree. Ancestral reconstructed sequences may have several desirable properties including increased stability, increased solubility, reduced sequence length, broader activity, and increased activity.

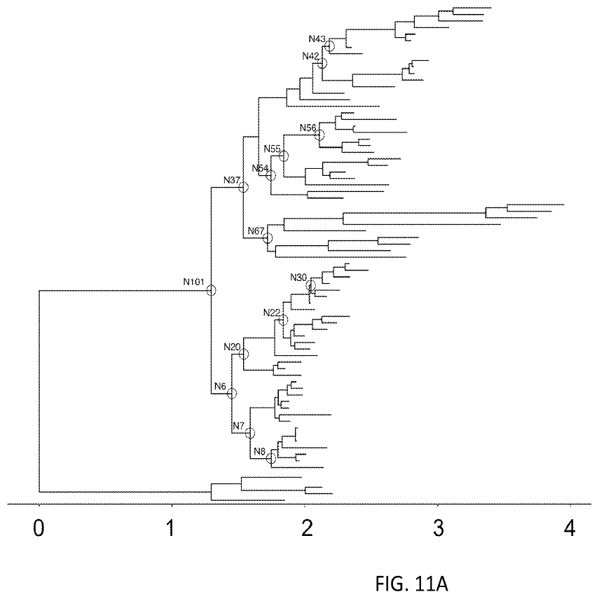

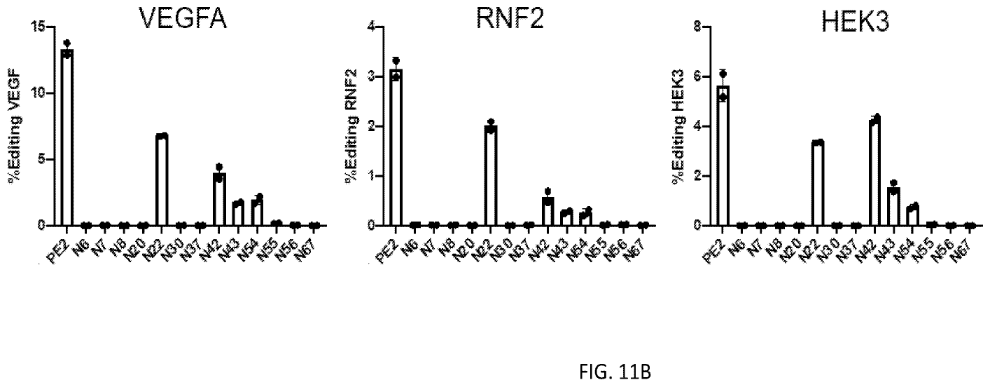

Using the phylogenetic tree constructed above of 12,061 sequences in the RT superfamily, the sequences and the phylogenetic sub-tree containing within the ZFERV family of retroviral RT, of which MMLV is a member, was isolated. The resulting sequence alignment was refined with MAFFT4. As shown in FIG. 11A, the tree was refined using the ZFERV specific alignment with the initial subtree as input. The evolutionary model described above (LG+R10+INV) was retained for the analysis. The resulting phylogenetic tree, evolutionary model, and alignment were used as input for the codeML algorithm, which is part of the PAML software package. Inferred ancestral sequences from codeML required additional inference of insertion and deletion (indels) events. The resulting sequences, starting tree, and alignment were used as input into RAxML for indel inference. Prime editors were constructed by replacing the MMLV-RT domain in the PE2 fusion protein (sequences shown in Table 15) with each of the identified ASR RTs in Table 3 (SEQ ID Nos 81-95). . The resulting constructs encoding the PE fusion proteins (Table 4A) were transfected into HEK293 cells along with PEgRNA targeting one of 3 genomic loci (HEK3, RNF2, or VEGFA) using the protocols described in EXAMPLE 1. As shown in FIG. 11B, several ZFERV ancestor sequences were found to be active in the PE editing context. Editing efficiencies achieved in the Prime Editing context with the active ASR RTs at the 3 genetic loci are reported in Table 4.

### Example 4—PE Fusion Protein with GB1 Domain

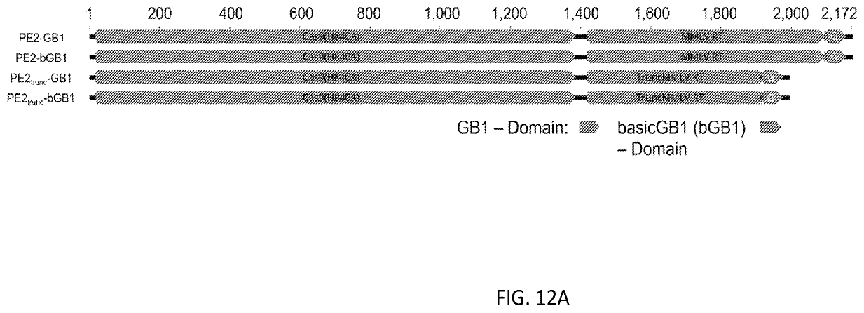

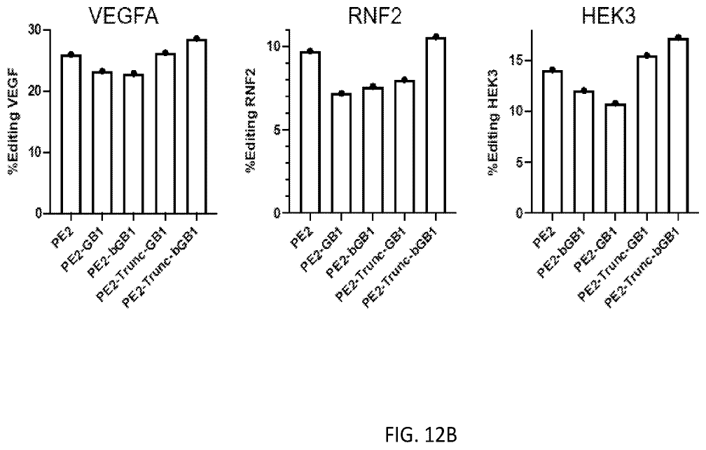

To examine the effect of prime editor solubility to editing efficiency, RNaseH domain of the MMLV-RT component of PE2 was removed by truncating the C-terminal portion of MMLV after amino acid residue D497 of SEQ ID NO: 504 (referred to herein as the “PE2d497RT”). Two solubility enhancement domains, GB1 and a basic variant of GB1 (bGB1), were used to generate PE variants. The bGB1 variant contains surface amino acid substitutions D22N, D36R, and E42K compared to the GB1 domain to reduce the number of acidic amino acids. Prime editor fusion proteins were constructed, from N terminus to C terminus, as follows: 1) PE2 N-terminal NLS-Cas9H840A-PE2 linker-PE2MMLVRT-GB1-PE2 C-terminal NLS; 2) PE2 N-terminal NLS-Cas9H840A-PE2 linker-PE2MMLVRT-bGB1-PE2 C-terminal NLS; 3) PE2 N-terminal NLS-Cas9H840A-PE2 linker-PE2d497RT-GB1-PE2 C-terminal NLS; or 4) PE2 N-terminal NLS-Cas9H840A-PE2 linker-PE2d497RT-bGB1-PE2 C-terminal NLS. Configuration of these PE variants are shown in FIG. 12A, and the sequences are provided in Table 6 (SEQ ID Nos 125-128).

Plasmids encoding the PE variants were transfected into HEK293 cells along with pegRNAs targeting 3 genetic loci (HEK3, RNF2 and VEGFA) as described in EXAMPLE 1. As shown in FIG. 12B, prime editing activities were observed for all four constructs. The PE2 with the truncated MMLV domain and the bGB1 domain showed the greatest overall activity with an average editing percentage of about 18.5% across three sites.

### Example 5— PE with Reverse Transcriptase Domain from Cas-RTs

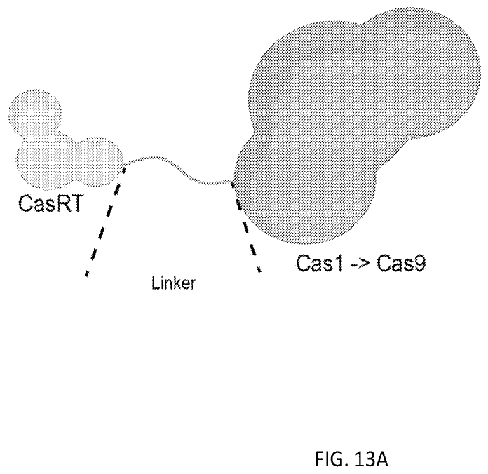

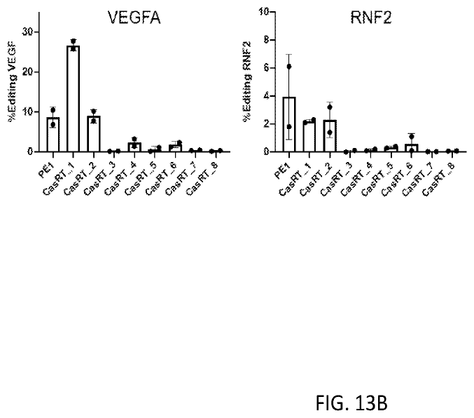

In some type III CRISPR systems, Cas1 is naturally fused to a reverse transcriptase, which may be referred to as a Cas-RT, and which may be used by bacteria to acquire spacers from RNA viruses. Exemplary Cas-RT domains are in Table 2 (CasRT_2 & CasRT_7), Tables 7, and 13. The Cas1 domain of various naturally occurring Cas1-RT fusion proteins was replaced at the conserved Cas1 domain boundary with a Cas9 domain, resulting in an engineered protein containing an spCas9 (H840A) nickase fused to the Cas1-RT domain and the endogenous linker sequence. A simplified schematic of the prime editor configuration is provided in FIG. 13A.

The resulting constructs were transfected into HEK293 cells targeting 2 genetic loci (RNF2 and VEGFA) using methods described in EXAMPLE 1. Two biological replicates for each site were collected. Initial results are shown in FIG. 13B; prime editing activities were observed in eight of out the nine constructs tested. Exemplary PE sequences with various Cas-RTs are listed in SEQ ID NO: 847-854 (Table 14). Additional experiments were performed with constructs made with the CasRT_2 and CasRT_6 RT domains (sequences in Table 2), the results of which are reported in the table 22 below.

TABLE 22
shows prime editing activities of prime editor constructs comprising CasRT_2 and
CasRT_6 RT domains

| Construct | Corresponding | Editing Efficiency (%) | | | | | Accession | | |
|---|---|---|---|---|---|---|---|---|---|
| Name | Cas | VEGFA | HEK3 | RNF2 | EMX1 | FANCF | No. | Species | Clade |
| CasRT_2 | SEQ ID | 9.2 | NA | 2.3 | NA | 6.8 | F2K1V9 | Marinomonas | CasRT |
| | NO. 209 | | | | | | | mediterranea | |
| CasRT_6 | SEQ ID | 2.1 | NA | 0.2 | NA | 3.2 | B3EIR7 | Chlorobium | CasRT |
| | NO. 210 | | | | | | | limicola | |

### Example 6—Identification of RT Families and Family Members

To identify RT subfamily members the phylogenetic tree and taxon sequences are separated into clades whereas a Glade is a group of sequences believed to have evolved from a common ancestor. Taxon sequences within clades are then searched for representee expert annotated sequences in the UniProt database. Primary sequence motifs are identified by finding sequence conservation blocks within the superfamily alignment. Sequences within clades are then searched for unique sequence motifs within the primary sequence conservation blocks to identify domain specific sequence motifs.

### Example 7—Installing a Desired Nucleotide Edit in a Cell Using Prime Editing Using Expression Vectors

A populations of host cells is transfected with a first vector that expresses a prime editor in the host cell and a second vector that expresses PEgRNA in the host cell. The PEgRNA has a nucleotide edit and a spacer sequence that bound to a pre-determined genomic location. One-week post-transfection, the population of host cells successfully transfected with the first and second vector are selected and clonally expanded. The individual host cell clone (targeting HEK3) is tested for being installed the nucleotide edit at the pre-determined genomic location using methods described in EXAMPLE 1. The high throughput sequencing step can also be replaced with Sanger Sequencing of the pre-determined genomic location.

### Example 8—Installing a Desired Nucleotide Edit in a Cell Using Prime Editing Using a Purified Prime Editor

A population of host cells is transfected or electroporated with an mRNA encoding the prime editor and a PEgRNA synthesized ex vivo. The PEgRNA has a nucleotide edit and a spacer sequence that bound to a pre-determined genomic location. One-week post-transfection, the population of host cells successfully transfected with the first and second vector are selected and clonally expanded. The individual host cell clone is tested for being installed the nucleotide edit at the pre-determined genomic location using methods described in EXAMPLE 1. The high throughput sequencing step can also be replaced with Sanger Sequencing of the pre-determined genomic location.

### Example 9—Installing a Desired Nucleotide Edit in a Cell Using Prime Editing Using Purified RNA

A population of host cells was transfected or electroporated with an mRNA encoding the prime editor and a PEgRNA synthesized ex vivo. The PEgRNA had a nucleotide edit and a spacer sequence that bound to a pre-determined genomic location. One-week post-transfection, the population of host cells successfully transfected with the first and second vector were selected and clonally expanded. The individual host cell clone was tested for being installed the nucleotide edit at the predetermined genomic location using methods described in EXAMPLE 1. The high throughput sequencing step can also be replaced with Sanger Sequencing of the pre-determined genomic location. A graph showing editing at the FANCF site with a SluCas9 prime editor and various PEgRNAs is shown in FIG. 4.

### Example 10— Evaluating Effect of Various Linkers on Prime Editor Efficiency

This example describes evaluation of the editing efficiency of prime editors comprising different linkers.

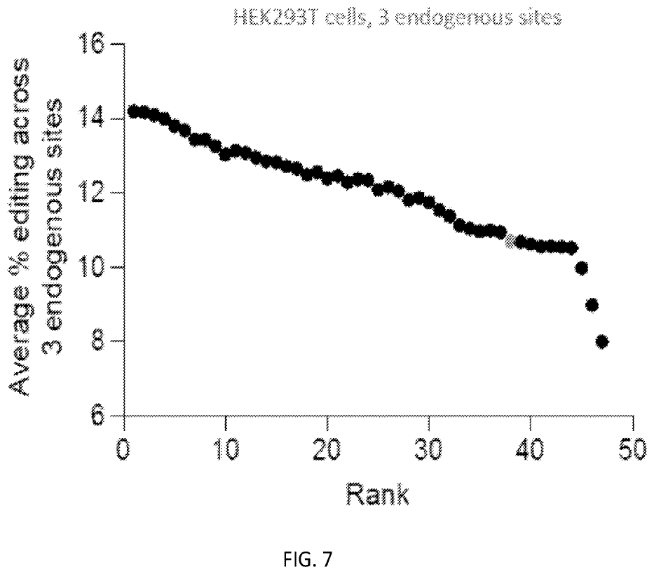

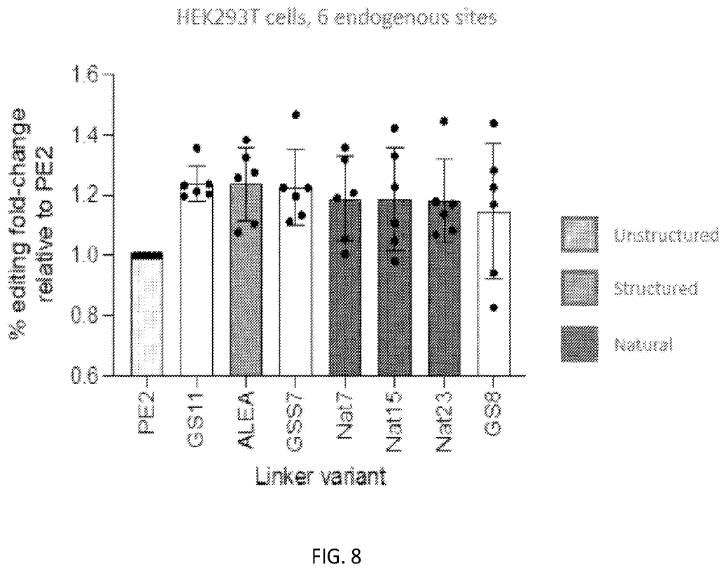

PE variants were constructed by replacing the linker in the PE2 fusion protein (sequences of components shown in Table 15) with each of the linkers in Table 11. Sequences of the linkers are provided in SEQ ID Nos 272-318. Plasmids encoding the PE variants were transfected into HEK293 cells along with pegRNAs targeting three different genomic loci using protocols described in Example 1. Activity of the 47 prime editors each having one of the linkers provided in SEQ ID Nos 272-318, calculated based on the average prime editing efficiency across the 3 endogenous sites were ranked and compared to editing efficiency of PE2, as shown in FIG. 7. Table 11 lists the activity of all 47 prime editors tested; editing efficiency at each of the three genomic loci and average fold increase over the efficiency of PE2 are reported. Of the 47 prime editors tested, seven were further examined by targeting six endogenous loci using the method described in Example 1. FIG. 8 shows the editing efficiency of these seven prime editors comprising in comparison to PE2.

### Example 11—Evaluating Effect of Various DNA Binding Domains on Prime Editor Efficiency

This example compares the editing efficiency of prime editors comprising different DNA binding domains.

Prime editors were constructed by replacing the SpCas9 nickase in the PE2 fusion protein (sequences of components shown in Table 15) with a SluCas9 nickase, a sRGN3.1 Cas9 nickase or sRGN3.3 Cas9 nickase. Sequences of the SluCas9 nickase, the sRGN3.1Cas9 nickase and the sRGN3.3Cas9 nickase are provided in SEQ ID Nos 496, 501, 502, and the PE sequences are provided in SEQ ID Nos 505, 511, and 512, respectively. Plasmids encoding the PE variants were transfected into HEK293 cells along with pegRNAs and ngRNA targeting genomic locus as indicated in Table 9 using protocols described in Example 1. One-week post-transfection, the population of successfully transfected host cells were selected and clonally expanded. The individual host cell clone was tested for being installed the nucleotide edit at the pre-determined genomic location using methods described in EXAMPLE 1. The results obtained are summarized in Table 9.

### INCORPORATION BY REFERENCE

All publications and patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the compositions and methods described herein. Such equivalents are intended to be encompassed by the following claims.

Lengthy table referenced here
US20240067940A1-20240229-T00001
Please refer to the end of the specification for access instructions.

Lengthy table referenced here
US20240067940A1-20240229-T00002
Please refer to the end of the specification for access instructions.

Lengthy table referenced here
US20240067940A1-20240229-T00003
Please refer to the end of the specification for access instructions.

Lengthy table referenced here
US20240067940A1-20240229-T00004
Please refer to the end of the specification for access instructions.

Lengthy table referenced here
US20240067940A1-20240229-T00005
Please refer to the end of the specification for access instructions.

Lengthy table referenced here
US20240067940A1-20240229-T00006
Please refer to the end of the specification for access instructions.

Lengthy table referenced here
US20240067940A1-20240229-T00007
Please refer to the end of the specification for access instructions.

Lengthy table referenced here
US20240067940A1-20240229-T00008
Please refer to the end of the specification for access instructions.

Lengthy table referenced here
US20240067940A1-20240229-T00009
Please refer to the end of the specification for access instructions.

Lengthy table referenced here
US20240067940A1-20240229-T00010
Please refer to the end of the specification for access instructions.

Lengthy table referenced here
US20240067940A1-20240229-T00011
Please refer to the end of the specification for access instructions.

Lengthy table referenced here
US20240067940A1-20240229-T00012
Please refer to the end of the specification for access instructions.

Lengthy table referenced here
US20240067940A1-20240229-T00013
Please refer to the end of the specification for access instructions.

Lengthy table referenced here
US20240067940A1-20240229-T00014
Please refer to the end of the specification for access instructions.

Lengthy table referenced here
US20240067940A1-20240229-T00015
Please refer to the end of the specification for access instructions.

Lengthy table referenced here
US20240067940A1-20240229-T00016
Please refer to the end of the specification for access instructions.

Lengthy table referenced here
US20240067940A1-20240229-T00017
Please refer to the end of the specification for access instructions.

Lengthy table referenced here
US20240067940A1-20240229-T00018
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240067940A1). An electronic copy of the table will also be available from the USPTO upon request and payment of the fee set forth in 37 CFR 1.19(b)(3).

What is claimed is:

1. A prime editing composition that comprises:
a) a DNA binding domain or a polynucleotide encoding the DNA binding domain; and
b) a DNA polymerase domain or a polynucleotide encoding the DNA polymerase domain, wherein the DNA polymerase domain comprises an amino acid sequence with at least 85% sequence identity to a sequence selected from the group consisting of SEQ ID NOs: 5, 6, 13, 15, 16, 17, 18, 21, 22, 130, 131, 204, 230, 232-244, 249-257, 261, 270, 271, 327, 329, 332, 333, 337, 340, 341, 342, 344, 489, 990-1006, 209, 210, 231, and 229.

2. The prime editing composition of claim 1, wherein the DNA polymerase domain comprises an amino acid sequence with at least 85% sequence identity to any one of sequences set forth in SEQ ID NOs: 209, 210, 229-244, 249-257, 261, 270, 271, 329, 990-1006.

3. The prime editing composition claim 1 or 2, wherein the amino acid sequence of the DNA polymerase domain has at least about 86%, 87%, 88%, 80%, 90%, 91%, 92%, 93%, 94%, 95%, 96%, 97%, 98%, 99%, or 100% sequence identity to the selected sequence.

4. The prime editing composition of any one of claims 1-3, wherein the selected sequence is SEQ ID NO: 261.

5. The prime editing composition of any one of claims 1-3, wherein the selected sequence for the DNA polymerase domain is SEQ ID NO:270.

6. The prime editing composition of any one of claims 1-3, wherein the selected sequence for the DNA polymerase domain is SEQ ID NO:16.

7. The prime editing composition of any one of claims 1-3, wherein the selected sequence for the DNA polymerase domain is SEQ ID NO:18.

8. The prime editing composition of any one of claims 1-7, wherein the DNA binding domain comprises a CRISPR associated (Cas) protein.

9. The prime editing composition of claim 8, wherein the Cas protein is a Type II Cas protein.

10. The prime editing composition of claim 9, wherein the Cas protein is a Cas9 protein

11. The prime editing composition of any one of claim 10, wherein the Cas9 protein is a nickase.

12. The prime editing composition of claim 11, wherein the Cas9 protein comprises a mutation in a HNH domain.

13. The prime editing composition of claim 8, wherein the Cas protein is a Type V Cas protein.

14. The prime editing composition of claim 13, wherein the Cas protein is a Cas12a, Cas12b, Cas12c, Cas12d, or Cas12e.

15. The prime editing composition of claim 13, wherein the Cas protein is a Cas12b.

16. The prime editing composition of any one of claims 1-15, wherein the DNA binding domain comprises an amino acid sequence with at least 85% sequence identity to a sequence selected from the group consisting of SEQ ID NOs: 138-146, 494, 858, 1100, 495-503, 1011, 1013.

17. The prime editing composition of claim 16, wherein the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence.

18. The prime editing composition of any one of claims 1-17, wherein the selected sequence for the DNA binding domain is SEQ ID NOs: 495-503, 1011, 1013, or 1100.

19. The prime editing composition of claim 18, wherein the selected sequence for the DNA binding domain is SEQ ID NOs: 495.

20. The prime editing composition of claim 18, wherein the selected sequence for the DNA binding domain is SEQ ID NOs: 496.

21. The prime editing composition of claim 18, wherein the selected sequence for the DNA binding domain is SEQ ID NOs: 501.

22. The prime editing composition of claim 18, wherein the selected sequence for the DNA binding domain is SEQ ID NO: 502.

23. The prime editing composition of any one of claims 1-22, wherein the DNA binding domain is connected to the DNA polymerase domain by a linker.

24. The prime editing composition of any one of claims 1-22, wherein the DNA binding domain is connected to the DNA polymerase domain by a peptide linker in a fusion protein.

25. The prime editing composition of claim 24, wherein the peptide linker comprises a sequence selected from the group consisting of SEQ ID NOs: 272-318, 1014.

26. The prime editing composition of claim 24 or 25, wherein the fusion protein comprises the DNA polymerase and the DNA binding domain from N-terminus to C-Terminus.

27. The prime editing composition of claim 24 or 25, wherein the fusion protein comprises the DNA binding and the DNA polymerase domain from N-terminus to C-Terminus.

28. The prime editing composition of any one of claims 1-27, wherein the DNA polymerase domain, the DNA binding domain, or both comprise one or more nuclear localization signals.

29. The prime editing composition of any one of claims 1-28, wherein the primer editing composition further comprises a solubility-enhancement (SET) domain.

30. The prime editing composition of claim 29, wherein the SET domain comprises an amino acids sequence selected from the group consisting of SEQ ID NOs: 96-124, 137.

31. The prime editing composition of any one of claims 1-30, wherein the sequence identities are determined by Needleman-Wunsch alignment of two protein sequences with Gap Costs set to Existence:
11 Extension: 1 where percent identity is calculated by dividing the number of identities by the length of the alignment.

32. A prime editing composition that comprises a fusion protein, or a polynucleotide encoding the fusion protein, wherein the fusion protein comprises a DNA binding domain and a DNA polymerization domain connected via a peptide linker, wherein the peptide linker comprises an amino acid sequence with at least 80% sequence identity to a sequence selected from the group consisting of SEQ ID NO: 273-318.

33. The prime editing composition of claim 32, wherein the amino acid sequence of the peptide linker has at least about 81%, 82%, 83%, 84%, 85%, 86%, 87%, 88%, 80%, 90%, 91%, 92%, 93%, 94%, 95%, 96%, 97%, 98%, 99%, or 100% sequence identity to the selected sequence.

34. The prime editing composition of claim 32 or 33, wherein the DNA polymerase domain comprises an amino acid sequence with at least 85% sequence identity to SEQ ID NO:856 or SEQ ID NO: 884.

35. The prime editing composition of claim 34, wherein the amino acid sequence of the DNA polymerase domain has at least about 86%, 87%, 88%, 80%, 90%, 91%, 92%, 93%, 94%, 95%, 96%, 97%, 98%, 99%, or 100% sequence identity to SEQ ID NO: 856.

36. The prime editing composition of any one of claims 32-35, wherein the Cas protein is a Type II Cas protein.

37. The prime editing composition of claim 36, wherein the Cas protein is a Cas9 protein

38. The prime editing composition of claim 37, wherein the Cas9 protein is a nickase.

39. The prime editing composition of claim 38, wherein the Cas9 protein comprises a mutation in a HNH domain.

40. The prime editing composition of any one of claims 32-35, wherein the Cas protein is a Type V Cas protein.

41. The prime editing composition of claim 37, wherein the Cas protein is a Cas12a, Cas12b, Cas12c, Cas12d, or Cas12e.

42. The prime editing composition of claim 41, wherein the Cas protein is a Cas12b.

43. The prime editing composition of any one of claims 32-42, wherein the DNA binding domain comprises an amino acid sequence with at least 85% sequence identity to a sequence selected from the group consisting of SEQ ID NOs: 138-146, 494, 858, 1100, 1011, 1013, 495-503.

44. The prime editing composition of claim 43, wherein the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence.

45. The prime editing composition of any one of claims 32-42, wherein the selected sequence for the DNA binding domain is SEQ ID NOs: 495-503, 1011, 1013, 1100.

46. The prime editing composition of claim 45, wherein the selected sequence for the DNA binding domain is SEQ ID NOs: 495.

47. The prime editing composition of claim 45, wherein the selected sequence for the DNA binding domain is SEQ ID NOs: 496.

48. The prime editing composition of claim 45, wherein the selected sequence for the DNA binding domain is SEQ ID NOs: 501.

49. The prime editing composition of claim 45, wherein the selected sequence for the DNA binding domain is SEQ ID NO: 502.

50. The prime editing composition of any one of claims 32-49, wherein the fusion protein comprises the DNA polymerase and the DNA binding domain from N-terminus to C-Terminus.

51. The prime editing composition of any one of claims 32-49, wherein the fusion protein comprises the DNA binding and the DNA polymerase domain from N-terminus to C-Terminus.

52. The prime editing composition of any one of claims 32-51, wherein the DNA polymerase domain, the DNA binding domain, or both comprise one or more nuclear localization signals.

53. The prime editing composition of any one of claims 32-52, wherein the primer editing composition further comprises a solubility-enhancement (SET) domain.

54. The prime editing composition of claim 53, wherein the SET domain comprises an amino acids sequence selected from the group consisting of SEQ ID NOs: 96-124, 137.

55. The prime editing composition of any one of claims 32-54, wherein the sequence identities are determined by Needleman-Wunsch alignment of two protein sequences with Gap Costs set to Existence: 11 Extension: 1 where percent identity is calculated by dividing the number of identities by the length of the alignment.

56. A prime editing composition that comprises:
a) a DNA binding domain, or a polynucleotide encoding the DNA binding domain, wherein the DNA binding domain comprises an amino acid sequence with at least 80% sequence identity to a sequence selected from the group consisting of SEQ ID NOs: 496, 501, 502, 1011, and 1013; and
b) a DNA polymerase domain or a polynucleotide encoding the DNA polymerase domain.

57. The prime editing composition of claim 56, wherein the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence.

58. The prime editing composition of claim 56 or 57, wherein the selected sequence for the DNA binding domain is SEQ ID NOs: 496.

59. The prime editing composition of claim 56 or 57, wherein the selected sequence for the DNA binding domain is SEQ ID NOs: 501.

60. The prime editing composition of claim 56 or 57, wherein the selected sequence for the DNA binding domain is SEQ ID NOs: 502.

61. The prime editing composition of any one of claims 56-60, wherein the DNA polymerase domain comprises an amino acid sequence with at least 85% sequence identity to SEQ ID NO:856 or SEQ ID NO: 884.

62. The prime editing composition of claim 61, wherein the amino acid sequence of the DNA polymerase domain has at least about 86%, 87%, 88%, 80%, 90%, 91%, 92%, 93%, 94%, 95%, 96%, 97%, 98%, 99%, or 100% sequence identity to SEQ ID NO: 856.

63. The prime editing composition of any one of claims 56-62, wherein the DNA binding domain is connected to the DNA polymerase domain by a linker.

64. The prime editing composition of any one of claims 56-62, wherein the DNA binding domain is connected to the DNA polymerase domain by a peptide linker in a fusion protein.

65. The prime editing composition of claim 64, wherein the peptide linker comprises a sequence selected from the group consisting of 272-318, 1014.

66. The prime editing composition of claim 64 or 65, wherein the fusion protein comprises the DNA polymerase and the DNA binding domain from N-terminus to C-Terminus.

67. The prime editing composition of claim 64 or 65, wherein the fusion protein comprises the DNA binding and the DNA polymerase domain from N-terminus to C-Terminus.

68. The prime editing composition of any one of claims 56-67, wherein the DNA polymerase domain, the DNA binding domain, or both comprise one or more nuclear localization signals.

69. The prime editing composition of any one of claims 56-67, wherein the primer editing composition further comprises a solubility-enhancement (SET) domain.

70. The prime editing composition of claim 69, wherein the SET domain comprises an amino acids sequence selected from the group consisting of SEQ ID NOs: 96-124, 137.

71. The prime editing composition of any one of claims 56-70, wherein the sequence identities are determined by Needleman-Wunsch alignment of two protein sequences with Gap Costs set to Existence: 11 Extension: 1 where percent identity is calculated by dividing the number of identities by the length of the alignment.

72. A prime editing composition that comprises:
a) a DNA binding domain or a polynucleotide encoding the DNA binding domain; and
b) a DNA polymerase domain, or a polynucleotide encoding the DNA polymerase domain, wherein the DNA polymerase domain comprises an amino acid sequence with at least 85% sequence identity to a sequence selected form the group consisting of SEQ ID NOs: 81, 91, 82, 84.

73. The prime editing composition of claim 72, wherein the amino acid sequence of the DNA polymerase domain has at least about 86%, 87%, 88%, 80%, 90%, 91%, 92%, 93%, 94%, 95%, 96%, 97%, 98%, 99%, or 100% sequence identity to the selected sequence.

74. The prime editing composition of claim 72 or 73, wherein the selected sequence for the DNA polymerase domain is SEQ ID NO: 81.

75. The prime editing composition of claim 72 or 73, wherein the selected sequence for the DNA polymerase domain is SEQ ID NO: 91

76. The prime editing composition of claim 72 or 73, wherein the selected sequence for the DNA polymerase domain is SEQ ID NO: 82.

77. The prime editing composition of claim 72 or 73, wherein the selected sequence for the DNA polymerase domain is SEQ ID NO: 84.

78. The prime editing composition of any one of claims 72-77, wherein the DNA binding domain comprises a CRISPR associated (Cas) protein.

79. The prime editing composition of claim 78, wherein the Cas protein is a Type II Cas protein.

80. The prime editing composition of claim 79, wherein the Cas protein is a Cas9 protein

81. The prime editing composition of claim 80, wherein the Cas9 protein is a nickase.

82. The prime editing composition of claim 81, wherein the Cas9 protein comprises a mutation in a HNH domain.

83. The prime editing composition of claim 82, wherein the Cas protein is a Type V Cas protein.

84. The prime editing composition of claim 83, wherein the Cas protein is a Cas12a, Cas12b, Cas12c, Cas12d, or Cas12e.

85. The prime editing composition of claim 83, wherein the Cas protein is a Cas12b.

86. The prime editing composition of any one of claims 72-85, wherein the DNA binding domain comprises an amino acid sequence with at least 85% sequence identity to a sequence selected from the group consisting of SEQ ID NOs: 138-146, 494, 858, 1100, 1011, 1013, 495-503.

87. The prime editing composition of claim 86, wherein the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence.

88. The prime editing composition of any one of claims 72-87, wherein the selected sequence for the DNA binding domain is SEQ ID NOs: 495-503, 1011, 1013, 1100.

89. The prime editing composition of claim 88, wherein the selected sequence for the DNA binding domain is SEQ ID NOs: 495.

90. The prime editing composition of claim 88, wherein the selected sequence for the DNA binding domain is SEQ ID NOs: 496.

91. The prime editing composition of claim 88, wherein the selected sequence for the DNA binding domain is SEQ ID NOs: 501.

92. The prime editing composition of claim 88, wherein the selected sequence for the DNA binding domain is SEQ ID NO:502.

93. The prime editing composition of any one of claims 72-92, wherein the DNA binding domain is connected to the DNA polymerase domain by a linker.

94. The prime editing composition of any one of claims 72-92, wherein the DNA binding domain is connected to the DNA polymerase domain by a peptide linker in a fusion protein.

95. The prime editing composition of claim 94, wherein the peptide linker comprises a sequence selected from the group consisting of 272-318, 1014.

96. The prime editing composition of claim 94 or 95, wherein the fusion protein comprises the DNA polymerase and the DNA binding domain from N-terminus to C-Terminus.

97. The prime editing composition of claim 94 or 95, wherein the fusion protein comprises the DNA binding and the DNA polymerase domain from N-terminus to C-Terminus.

98. The prime editing composition of any one of claims 72-97, wherein the DNA polymerase domain, the DNA binding domain, or both comprise one or more nuclear localization signals.

99. The prime editing composition of any one of claims 72-98, wherein the primer editing composition further comprises a solubility-enhancement (SET) domain.

100. The prime editing composition of claim 99, wherein the SET domain comprises an amino acids sequence selected from the group consisting of SEQ ID NOs: 96-124, 137.

101. The prime editing composition of any one of claims 72-100, wherein the sequence identities are determined by Needleman-Wunsch alignment of two protein sequences with Gap Costs set to Existence: 11 Extension: 1 where percent identity is calculated by dividing the number of identities by the length of the alignment.

102. A prime editing composition comprising
a) a DNA binding domain or a polynucleotide encoding the DNA binding domain, and
b) a reverse transcriptase (RT) domain or a polynucleotide encoding the RT domain, wherein the RT domain is from a naturally occurring fusion between a Type III CRISPR system protein and a reverse transcriptase, and wherein the DNA binding domain is heterologous to the RT domain.

103. The prime editing composition claim 102, wherein the RT domain is from a naturally occurring Cas1-RT fusion protein.

104. The prime editing composition of claim 102 or 103, wherein the RT domain comprises an amino acid sequence with at least 85% sequence identity to a sequence selected from the group consisting of SEQ ID NOs: 345, 129-136, 396, 533-846.

105. The prime editing composition of claim 104, wherein the amino acid sequence of the RT domain has at least about 86%, 87%, 88%, 80%, 90%, 91%, 92%, 93%, 94%, 95%, 96%, 97%, 98%, 99%, or 100% sequence identity to the selected sequence.

106. The prime editing composition of claim 104 or 105, wherein the selected sequence for the RT domain is SEQ ID NO: 209.

107. The prime editing composition of claim 104 or 105, wherein the selected sequence for the RT domain is SEQ ID NO: 210.

108. The prime editing composition of claim 106 or 107, wherein the RT domain is fused directly to the DNA binding domain.

109. The prime editing composition of claim 108, wherein the RT domain is fused to the N-terminus of the DNA binding domain.

110. The prime editing composition of claim 108, wherein the RT domain is fused to the C-terminus of the DNA binding domain.

111. The prime editing composition of claim any one of claims 102-110, wherein the DNA binding domain comprises a CRISPR associated (Cas) protein.

112. The prime editing composition of claim 111, wherein the Cas protein is a Type II Cas protein.

113. The prime editing composition of claim 112, wherein the Cas protein is a Cas9 protein

114. The prime editing composition of claim 113, wherein the Cas9 protein is a nickase.

115. The prime editing composition of claim 114, wherein the Cas9 protein comprises a mutation in a HNH domain.

116. The prime editing composition of claim 111, wherein the Cas protein is a Type V Cas protein.

117. The prime editing composition of claim 116, wherein the Cas protein is a Cas12a, Cas12b, Cas12c, Cas12d, or Cas12e.

118. The prime editing composition of claim 117, wherein the Cas protein is a Cas12b.

119. The prime editing composition of any one of claims 102-118, wherein the DNA binding domain comprises an amino acid sequence with at least 85% sequence identity to a sequence selected from the group consisting of SEQ ID NOs: 138-146, 494, 858, 1100, 1011, 1013, 495-503.

120. The prime editing composition of claim 119, wherein the amino acid sequence of the DNA binding domain has at least about 86%, 87%, 88%, 80%, 90%, 91%, 92%, 93%, 94%, 95%, 96%, 97%, 98%, 99%, or 100% sequence identity to the selected sequence.

121. The prime editing composition of any one of claims 119-120, wherein the selected sequence for the DNA binding domain is SEQ ID NOs: 495-503, 1100, 1011, 1013.

122. The prime editing composition of claim 121, wherein the selected sequence for the DNA binding domain is SEQ ID NOs: 1011.

123. The prime editing composition of claim 121, wherein the selected sequence for the DNA binding domain is SEQ ID NOs: 1013

124. The prime editing composition of claim 121, wherein the selected sequence for the DNA binding domain is SEQ ID NOs: 496.

125. The prime editing composition of claim 121, wherein the selected sequence for the DNA binding domain is SEQ ID NOs: 501.

126. The prime editing composition of claim 121, wherein the selected sequence for the DNA binding domain is SEQ ID NO: 502.

127. The prime editing composition of any one of claims 102-126, wherein the RT domain, the DNA binding domain, or both comprise one or more nuclear localization signals.

128. The prime editing composition of any one of claims 102-127, wherein the primer editing composition further comprises a solubility-enhancement (SET) domain.

129. The prime editing composition of claim 128, wherein the SET domain comprises an amino acids sequence selected from the group consisting of SEQ ID NOs: 96-124, 137.

130. The prime editing composition of any one of claims 102-129, wherein the sequence identities are determined by Needleman-Wunsch alignment of two protein sequences with Gap Costs set to Existence: 11 Extension: 1 where percent identity is calculated by dividing the number of identities by the length of the alignment.

131. A prime editing composition that comprises:
a) a DNA polymerase domain or a polynucleotide encoding the DNA polymerase domain, wherein the DNA polymerase domain comprises an amino acid sequence having at least 85% identity to SEQ ID NO: 856 or 884.
b) a DNA binding domain or a polynucleotide encoding the DNA binding domain, wherein the DNA binding domain comprises an amino acid sequence having at least 85% identity to SEQ ID NO: 1011 or 1013; and
c) a solubility-enhancement (SET) domain or a polynucleotide encoding the SET domain, wherein the SET domain comprises an amino acid sequence with at least 80% sequence identity to an amino acid sequence selected from the group consisting of SEQ ID NOs: 96-124, 137.

132. The prime editing composition of claim 131, wherein the amino acid sequence for the SET domain has at least 81%, 82%, 83%, 84%, 85%, 86%, 87%, 88%, 80%, 90%, 91%, 92%, 93%, 94%, 95%, 96%, 97%, 98%, 99%, or 100% sequence identity to the selected sequence.

133. The prime editing composition of claim 131 or 132, wherein the selected sequence for the SET domain is SEQ ID NO: 102.

134. The prime editing composition of claim 131 or 132, wherein the selected sequence for the SET domain is SEQ ID NO: 137

135. The prime editing composition of any one of claims 131-134, wherein the amino acid sequence for the DNA polymerase domain has at least 86%, 87%, 88%, 80%, 90%, 91%, 92%, 93%, 94%, 95%, 96%, 97%, 98%, 99%, or 100% sequence identity to the selected sequence.

136. The prime editing composition of claim 135, wherein the selected sequence for the DNA polymerase domain is 856.

137. The prime editing composition of claim 135, wherein the selected sequence for the DNA polymerase domain is 884.

138. The prime editing composition of any one of claims 131-137, wherein the amino acid sequence for the DNA binding domain has at least 86%, 87%, 88%, 80%, 90%, 91%, 92%, 93%, 94%, 95%, 96%, 97%, 98%, 99%, or 100% sequence identity to the selected sequence.

139. The prime editing composition of claim 135, wherein the selected sequence for the DNA binding domain is SEQ ID NO: 1011.

140. The prime editing composition of claim 135, wherein the selected sequence for the DNA binding domain is SEQ ID NO: 1013.

141. The prime editing composition of any one of claims 131-140, wherein the SET domain is fused to the DNA polymersase via an SGGS linker.

142. The prime editing composition of any one of claims 131-141, wherein the DNA binding domain is connected to the DNA polymerase domain by a linker.

143. The prime editing composition of any one of claims 131-142, wherein the DNA binding domain is connected to the DNA polymerase domain by a peptide linker in a fusion protein.

144. The prime editing composition of claim 143, wherein the peptide linker comprises a sequence selected from the group consisting of SEQ ID NOs: 272-318, 1014.

145. The prime editing composition of claim 143 or 144, wherein the fusion protein comprises the DNA polymerase and the DNA binding domain from N-terminus to C-Terminus.

146. The prime editing composition of claim 143 or 144, wherein the fusion protein comprises the DNA binding and the DNA polymerase domain from N-terminus to C-Terminus.

147. The prime editing composition of any one of claims 131-146, wherein the DNA polymerase domain, the DNA binding domain, the SET domain, or a combination thereof comprise one or more nuclear localization signals.

148. The prime editing composition of claim 143 or 144, wherein the fusion protein comprises a nuclear localization signal, the DNA binding domain, the peptide linker, the DNA polymerase domain, the SGGS linker, the SET domain, and a second nuclear localization signal from N-terminus to C-terminus.

149. The prime editing composition of any one of claims 131-148, wherein the sequence identities are determined by Needleman-Wunsch alignment of two protein sequences with Gap Costs set to Existence: 11 Extension: 1 where percent identity is calculated by dividing the number of identities by the length of the alignment.

150. The prime editing composition of any one of claims 1-149, further comprising a prime editing guide RNA (PEgRNA), or a polynucleotide encoding the PEgRNA.

151. The prime editing composition of any one of claims 1-150, further comprising a nick guide RNA (ngRNA), or a polynucleotide encoding the ngRNA.

152. A vector comprising one or more of the polynucleotides of the prime editing compositions of any one of claims 1-149.

153. The vector of claim 152, wherein the vector is a AAV vector.

154. The vector of claim 152, wherein the vector is an lipid nanoparticle (LNP).

155. A pharmaceutical composition comprising the prime editing composition of any one of claims 1-151, or the vector of any one of claims 152-154.

156. The pharmaceutical composition of claim 155, further comprising a pharmaceutically acceptable excipient.

157. An engineered reverse transcriptase (RT) that comprises an amino acid sequence with at least 85% sequence identity to an amino acid sequence selected from the group consisting of SEQ ID NOs: 81-95.

158. The engineered RT of claim 150, wherein the amino acid sequence for the engineered RT has at least 86%, 87%, 88%, 80%, 90%, 91%, 92%, 93%, 94%, 95%, 96%, 97%, 98%, 99%, or 100% sequence identity to the selected sequence.

159. The engineered RT of claim 150 or 151, wherein the selected sequence for the engineered RT is SEQ ID NO: 84.

160. The engineered RT of claim 150 or 151, wherein the selected sequence for the engineered RT is SEQ ID NO: 82.

161. The engineered RT of claim 150 or 151, wherein the selected sequence for the engineered RT is SEQ ID NO: 81

162. The engineered RT of claim 150 or 151, wherein the selected sequence for the engineered RT is SEQ ID NO: 91

163. The engineered RT of any one of claims 150-155, wherein the sequence identities are determined by Needleman-Wunsch alignment of two protein sequences with Gap Costs set to Existence: 11 Extension: 1 where percent identity is calculated by dividing the number of identities by the length of the alignment.

164. A prime editing composition that comprises a fusion protein, or a polynucleotide encoding the fusion protein, wherein the fusion protein comprises a DNA binding domain and a DNA polymerization domain connected via a peptide linker, wherein the fusion protein comprises an amino acid sequence with at least 80% sequence identity to a sequence selected from the group consisting of SEQ ID NO: 504, 939-987, 1011, 1012, 1013, 1007-1010, 504-513, 514-521.

165. The prime editing composition claim 164, wherein the amino acid sequence of the DNA polymerase domain has at least about 86%, 87%, 88%, 80%, 90%, 91%, 92%, 93%, 94%, 95%, 96%, 97%, 98%, 99%, or 100% sequence identity to the selected sequence.

166. The prime editing composition of any one of claims 164-165, wherein the selected sequence is SEQ ID NO: 940.

167. The prime editing composition of any one of claims 164-165, wherein the selected sequence is SEQ ID NO: 941.

168. The prime editing composition of any one of claims 164-165, wherein the selected sequence is SEQ ID NO: 976.

169. The prime editing composition of any one of claims 164-165, wherein the selected sequence is SEQ ID NO: 977.

170. The prime editing composition of any one of claims 164-165, wherein the selected sequence is SEQ ID NO: 505.

171. The prime editing composition of any one of claims 164-165, wherein the selected sequence is SEQ ID NO: 511.

172. The prime editing composition of any one of claims 164-165, wherein the selected sequence is SEQ ID NO: 512.

173. The engineered RT of any one of claims 150-155, wherein the sequence identities are determined by Needleman-Wunsch alignment of two protein sequences with Gap Costs set to Existence: 11 Extension: 1 where percent identity is calculated by dividing the number of identities by the length of the alignment.

174. A vector comprising one or more of the polynucleotides of the prime editing compositions of any one of claims 164-173.

175. The vector of claim 174, wherein the vector is a AAV vector.

176. The vector of claim 175, wherein the vector is an lipid nanoparticle (LNP).

177. A pharmaceutical composition comprising the prime editing composition of any one of claims 164-173, or the vector of any one of claims 174-176.
